(19) United States
(12) Patent Application Publication
(10) Pub. No.: US 2025/0177153 A1
(43) Pub. Date: Jun. 5, 2025

(54) STEMLESS REVERSE HUMERAL IMPLANT SYSTEMS

(71) Applicant: Encore Medical, L.P. (d/b/a/ DJO Surgical), Austin, TX (US)

(72) Inventors: Joseph A. Abboud, Philadelphia, PA (US); Thomas Bradley Edwards, Houston, TX (US); Mark A. Frankle, Tampa, FL (US); Joseph P. Iannotti, Cleveland, OH (US); Jonathan Levy, Overland Park, FL (US); Gerald Williams, Philadelphia, PA (US); Stuart L. Axelson, JR., Succasunna, NJ (US); Alysha Robicheaux, Austin, TX (US); Sergio Gutierrez, Tampa, FL (US)

(21) Appl. No.: 18/705,415

(22) PCT Filed: Oct. 26, 2022
(86) PCT No.: PCT/US2022/047896

§ 371 Date: Apr. 26, 2024

Related U.S. Application Data
(60) Provisional application No. 63/272,880, filed on Oct. 28, 2021.

(51) Int. Cl. A61F 2/40

(52) U.S. Cl. CPC A61F 2/4003; A61F 2/4014

(57) ABSTRACT

A humeral component of a prosthetic assembly that is used in repairing or reconstructing a joint between a humerus and a scapula that includes a stemless portion and a liner portion. The liner portion is attached to the stemless portion and adapted to receive a ball portion of a cooperating “reverse” type glenoid component.

### CROSS-REFERENCE TO RELATED APPLICATIONS

This application claims priority to, and benefit of, U.S. Provisional Patent Application No. 63/272,880 filed Oct. 28, 2021, which is hereby incorporated by reference herein its entirety.

### FIELD OF DISCLOSURE

This disclosure relates generally to orthopedic prostheses for use in reconstruction of a human shoulder joint. More particularly, the disclosure relates to such a prosthesis that is adapted for use in repairing or reconstructing an unstable shoulder joint, and where it is desirable to preserve as much metaphyseal bone as possible.

### BACKGROUND OF THE DISCLOSURE

The human shoulder joint provides the broadest range of motion of any joint in the body and consists of the proximal end of the humerus or the humeral head, and it articulates with a structure called the glenoid fossa on the lateral end of the scapula. The ability to have the full range of glenohumeral motion is predicated on the stabilization of the head of the humerus on the glenoid fossa by a functioning rotator cuff, consisting of the supraspinatus, infraspinatus, subscapularis and teres minor, and the static stabilizers, consisting of the capsule, labrum, glenohumeral ligaments, and the negative intraarticular pressure.

When the shoulder joint becomes unstable with superior escape of the humeral head due to rotator cuff tear arthropathy, a failed hemi-arthroplasty, or other conditions where the joint becomes unbalanced leading to asymmetric glenoid wear, the shoulder joint will need to be reconstructed with a reverse shoulder prosthesis. Such reconstruction can restore some degree of range of motion to the unstable joint by stabilizing the center of rotation of the humerus and allowing the deltoid muscle to more efficiently elevate the humerus, thus allowing some basic activities of daily living to be performed. Current reverse shoulder prostheses consist of a humeral component with a stem that fits down the intramedullary canal which limits placement of the implant. In contrast, a stemless reverse allows more variability in placement on the surgical cut, as well as decreasing the amount of bone that has to be removed.

It is therefore desirable if an implant were available that would provide a reverse shoulder prosthesis without the stem portion. It would also be desirable if an implant could be provided that would allow repair of the joint while reducing the amount of bone needing to be removed from the intramedullary canal. It would also be desirable if such an implant could be provided that would allow for adding bone substitute material when there is a lack of adequate bone quality in the proximal end of the humerus.

Among the advantages of the preferred embodiments of the disclosure is that they provide a prosthetic component that can be implanted with minimal bone removal. Another advantage of the preferred embodiments of the disclosure is that they provide a prosthesis that has a greater freedom of placement on the surgical cut of the humeral head. Still another advantage of the preferred embodiments of the disclosure is that they provide a prosthesis that can be used to repair a shoulder joint without the need for adding bone graft material when there is a lack of adequate bone quality in the proximal end of the humerus. Yet another advantage of the preferred embodiments of the disclosure is that they allow for placement of the prosthesis in an inlay fashion to reduce off-axis forces being place on the implant.

Additional objects and advantages of this disclosure will become apparent from an examination of the drawings and the ensuing description.

### SUMMARY

In some embodiments, the disclosed systems, methods, and apparatuses disclosed herein comprise a humeral component of a prosthetic assembly for use in repairing or reconstructing a joint between a humerus and a scapula. Disclosure. In some embodiments, an apparatus accepts a cup that interfaces with a ball portion attached to a glenoid component that is adapted for attachment to the scapula. The humeral component comprises a stemless humeral portion and a cup portion. The cup portion is adapted to receive the ball portion of the glenoid component and is attached to the stemless humeral portion.

In order to facilitate an understanding of the disclosure, the preferred embodiments of the disclosure are illustrated in the drawings, and a detailed description thereof follows. It is not intended, however, that the disclosure be limited to the particular embodiments described or to use in connection with the apparatus illustrated herein. Various modifications and alternative embodiments such as would ordinarily occur to one skilled in the art to which the disclosure relates are also contemplated and included within the scope of the disclosure described and claimed herein.

### BRIEF DESCRIPTION OF THE DRAWINGS

The presently preferred embodiments of the disclosure are illustrated in the accompanying drawings, in which like reference numerals represent like parts throughout, and in which:

FIG. 1 is an illustration of the anterior view of the bones of a normal human shoulder joint.

FIG. 2 is an illustration of the anterior view of a human shoulder joint with a reverse shoulder implant.

FIG. 3 is a front perspective view of a first embodiment of the disclosure.

FIG. 4 is a rear perspective view of the first embodiment of the disclosure.

FIG. 5 is a rear view of the first embodiment of the disclosure.

FIG. 6 is an exploded view of the first embodiment of the disclosure.

FIG. 7 is a front perspective view of a first embodiment of the disclosure.

FIG. 8 is a rear perspective view of the second embodiment of the disclosure.

FIG. 9 is a rear view of the second embodiment of the disclosure.

FIG. 10 is an exploded view of the second embodiment of the disclosure.

FIG. 11 is a front perspective view of a third embodiment of the disclosure.

FIG. 12 is a rear perspective view of the third embodiment of the disclosure.

FIG. 13 is a rear view of the third embodiment of the disclosure.

FIG. 14 is an exploded view of the third embodiment of the disclosure.

FIG. 15 is a front perspective view of a fourth embodiment of the disclosure.

FIG. 16 is a rear perspective view of the fourth embodiment of the disclosure.

FIG. 17 is a rear view of the fourth embodiment of the disclosure.

FIG. 18 is an exploded view of the fourth embodiment of the disclosure.

FIG. 19 is a front perspective view of a fifth embodiment of the disclosure.

FIG. 20 is a rear perspective view of the fifth embodiment of the disclosure.

FIG. 21 is a rear view of the fifth embodiment of the disclosure.

FIG. 22 is an exploded view of the fifth embodiment of the disclosure.

FIG. 23 is a sectioned side view of the fifth embodiment of the disclosure.

FIG. 24 shows various views of the sixth embodiment of the disclosure.

FIG. 25 is an exploded view of the sixth embodiment of the disclosure.

FIG. 26 is a front perspective view of a sixth embodiment of the disclosure.

FIG. 27 is a rear perspective view of the sixth embodiment of the disclosure.

FIG. 28 is an exploded view of the sixth embodiment of the disclosure.

FIG. 29 is a front perspective view of a seventh embodiment of the disclosure.

FIG. 30 is a side view of the seventh embodiment of the disclosure.

FIG. 31 is a rear perspective view of the seventh embodiment of the disclosure.

FIG. 32 is an exploded view of the seventh embodiment of the disclosure.

FIG. 33 is a front perspective view of an eighth embodiment of the disclosure.

FIG. 34 is a rear perspective view of the eighth embodiment of the disclosure.

FIG. 35 is an exploded view of the eighth embodiment of the disclosure.

FIG. 36 is a front perspective view of a ninth embodiment of the disclosure.

FIG. 37 is a rear perspective view of the ninth embodiment of the disclosure.

FIG. 38 is an exploded view of the ninth embodiment of the disclosure.

FIG. 39 is a front perspective view of a tenth embodiment of the disclosure.

FIG. 40 is an exploded view of the tenth embodiment of the disclosure.

FIG. 41 is a front perspective view of an eleventh embodiment of the disclosure.

FIG. 42 is a rear perspective view of the eleventh embodiment of the disclosure.

FIG. 43 is an exploded view of the eleventh embodiment of the disclosure.

FIG. 44 is a front perspective view of a twelfth embodiment of the disclosure.

FIG. 45 is a rear perspective view of the twelfth embodiment of the disclosure.

FIG. 46 is an exploded view of the twelfth embodiment of the disclosure.

FIG. 47 shows various alternative views of the twelfth embodiment of the disclosure.

FIG. 48 shows additional alternative views of the twelfth embodiment of the disclosure.

FIG. 49 is a view showing alternative positions of the twelfth embodiment of the disclosure.

FIG. 50 is a front perspective view of a thirteenth embodiment of the disclosure.

FIG. 51 is a rear perspective view of the thirteenth embodiment of the disclosure.

FIG. 52 is an exploded front perspective view of the thirteenth embodiment of the disclosure.

FIG. 53 is an exploded rear perspective view of the thirteenth embodiment of the disclosure.

FIG. 54 is a front perspective view of a fourteenth embodiment of the disclosure.

FIG. 55 is a rear perspective view of the fourteenth embodiment of the disclosure.

FIG. 56 is an exploded front perspective view of the fourteenth embodiment of the disclosure.

FIG. 57 is an exploded rear perspective view of the fourteenth embodiment of the disclosure.

FIG. 58 is a front perspective view of a fifteenth embodiment of the disclosure.

FIG. 59 is a rear perspective view of the fifteenth embodiment of the disclosure.

FIG. 60 is an exploded front perspective view of the fifteenth embodiment of the disclosure.

FIG. 61 is an exploded rear perspective view of the fifteenth embodiment of the disclosure.

FIG. 62 is a front perspective view of a sixteenth embodiment of the disclosure.

FIG. 63 is a rear perspective view of the sixteenth embodiment of the disclosure.

FIG. 64 is an exploded front perspective view of the sixteenth embodiment of the disclosure.

FIG. 65 is an exploded rear perspective view of the sixteenth embodiment of the disclosure.

FIG. 66 is a front perspective view of a seventeenth embodiment of the disclosure.

FIG. 67 is a rear perspective view of the seventeenth embodiment of the disclosure.

FIG. 68 is an exploded front perspective view of the seventeenth embodiment of the disclosure.

FIG. 69 is another exploded front perspective view of the seventeenth embodiment of the disclosure.

FIG. 70 is a front perspective view of an eighteenth embodiment of the disclosure.

FIG. 71 is a rear perspective view of the eighteenth embodiment of the disclosure.

FIG. 72 is an exploded front perspective view of the eighteenth embodiment of the disclosure.

FIG. 73 is a front perspective view of a nineteenth embodiment of the disclosure.

FIG. 74 is a rear perspective view of the nineteenth embodiment of the disclosure.

FIG. 75 is a side perspective view of the nineteenth embodiment of the disclosure.

FIG. 76 is an exploded rear perspective view of the nineteenth embodiment of the disclosure.

FIG. 77 is a front perspective view of a twentieth embodiment of the disclosure.

FIG. 78 is another front perspective view of the twentieth embodiment of the disclosure.

FIG. 79 is a rear perspective view of the twentieth embodiment of the disclosure.

FIG. 80 is an exploded front perspective view of the twentieth embodiment of the disclosure.

FIG. 81 is a front perspective view of the twenty-first embodiment of the disclosure.

FIG. 82 is a rear perspective view of the twenty-first embodiment of the disclosure.

FIG. 83 is a rear view of the twenty-first embodiment of the disclosure.

FIG. 84 is a front perspective view of the twenty-second embodiment of the disclosure.

FIG. 85 is a rear perspective view of the twenty-second embodiment of the disclosure.

FIG. 86 is a rear view of the twenty-second embodiment of the disclosure.

FIG. 87 is an exploded front perspective view of the twenty-second embodiment of the disclosure.

FIG. 88 is a front perspective view of the twenty-third embodiment of the disclosure.

FIG. 89 is a rear perspective view of the twenty-third embodiment of the disclosure.

FIG. 90 is a rear view of the twenty-third embodiment of the disclosure.

FIG. 91 is an exploded front perspective view of the twenty-third embodiment of the disclosure.

### DETAILED DESCRIPTION

The following detailed description and the appended drawings describe and illustrate various exemplary humeral prostheses, methods, and components. The description and drawings are exemplary in nature and are provided to enable one skilled in the art to make and use one or more exemplary humeral prostheses and/or components, and/or practice one or more exemplary methods. They are not intended to limit the scope of the claims in any manner.

The use of “e.g.,” “etc.,” “for instance,” “in example,” and “or” and grammatically related terms indicates non-exclusive alternatives without limitation, unless otherwise noted. The use of “optionally” and grammatically related terms means that the subsequently described element, event, feature, or circumstance may or may not be present/occur, and that the description includes instances where said element, event, feature, or circumstance occurs and instances where it does not. The use of “exemplary” refers to “an example of” and is not intended to convey a meaning of an ideal or preferred embodiment. The use of “attached” and “coupled” grammatically related terms refers to the fixed, releasable, or integrated association of two or more elements and/or devices with or without one or more other elements in between. Thus, the term “attached” or “coupled”, and grammatically related terms includes releasably attaching or fixedly attaching two or more elements and/or devices in the present or absence of one or more other elements in between. As used herein, the terms “proximal” and “distal” are used to describe opposing axial ends of the particular elements or features being described in relation to anatomical placement.

While the systems, methods, and components described herein are exemplified by systems and methods for humeral prostheses, the systems, methods, and components described and illustrated herein can be used to treat any suitable ailment or joint within the body of an animal, including, but not limited to, humans. Skilled artisans will be able to select a suitable ailment and/or joint within the body of an animal to utilize a system and/or method described herein according to a particular embodiment based on various considerations, including the type of ailment and/or the structural arrangement at a treatment site. Example joints considered suitable to utilize a system, method, and/or component described herein include, but are not limited to, the shoulder joint, the elbow joint, the knee joint, the hip joint, and the ankle joint.

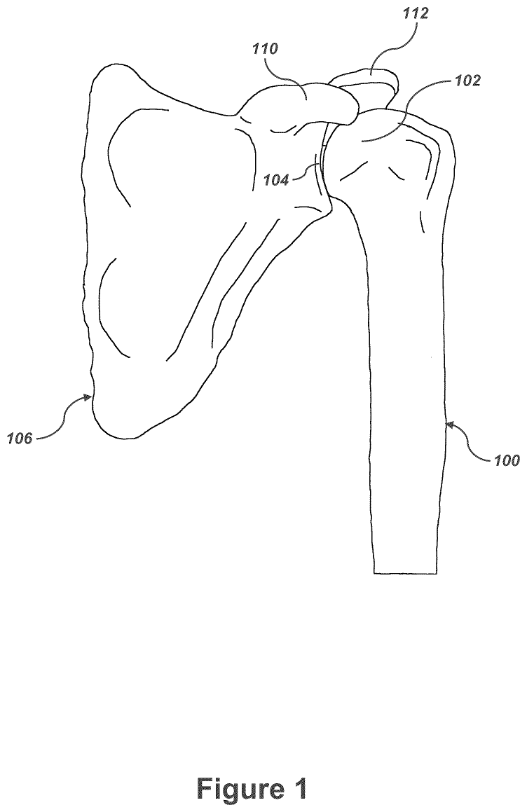

FIG. 1 is an anterior view of the bones of a normal human shoulder joint. As shown therein, the humerus 100 includes the humeral head 102 that forms the “ball” portion of this ball-and-socket joint. The humeral head 102 is in contact with the glenoid fossa 104 of scapula 106 which forms the “socket” portion of the shoulder joint. Additional structures of the scapula include the coracoid process 110, and the acromion 112. The humerus is held in place against the scapula by passive suspension with the aid of various ligaments (not shown) and active suspension in which the rotator cuff muscles, including the supraspinatus, infraspinatus, Teres Minor and Subscapularis, help to balance the head of the humerus in the glenoid socket against the upward pull of the deltoid muscle.

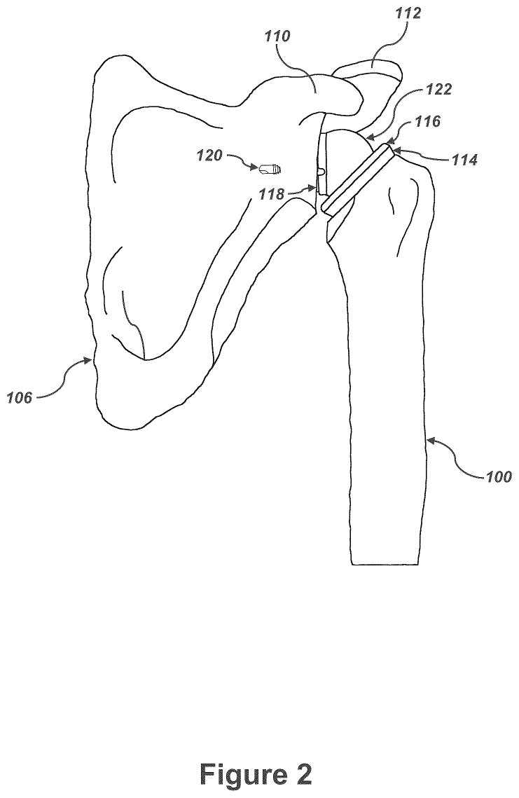

Arthritis, bone disease or injury can alter the smooth functioning of the shoulder joint. In primary joint repair, the head of the humerus is typically removed and a prosthetic stem is implanted into the humerus. A prosthetic ball is attached to this stem and a prosthetic socket is implanted in the glenoid fossa so as to articulate with the prosthetic ball. However, some conditions can make repair or reconstruction that maintains the normal spatial relationship between the “ball” and the “socket” of the joint impossible. Under these circumstances, repair or reconstruction of the joint using a “reverse” prosthesis, as seen in FIG. 2, employs a stemless shell 114 and a liner 116. Preferably, the stemless shell 114 is comprised of titanium or another biocompatible metal, and the liner 116 is comprised of plastic, ceramic, or another biocompatible material. On the glenoid side, a baseplate 118 is adapted to be attached to the glenoid portion of the scapula 106 using a plurality of fasteners 120. The ball 122 is adapted to attach to the baseplate 118, and it articulates with the liner 116 that attaches to the stemless shell 114, as shown in FIG. 2.

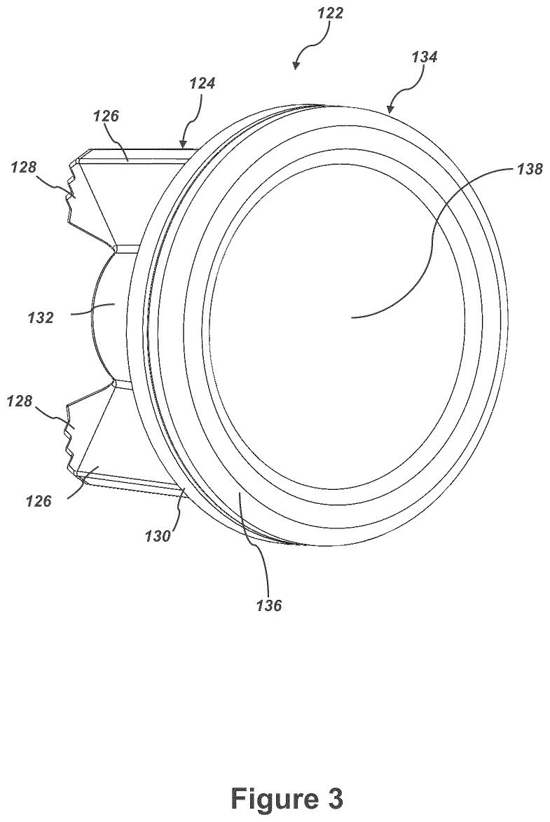

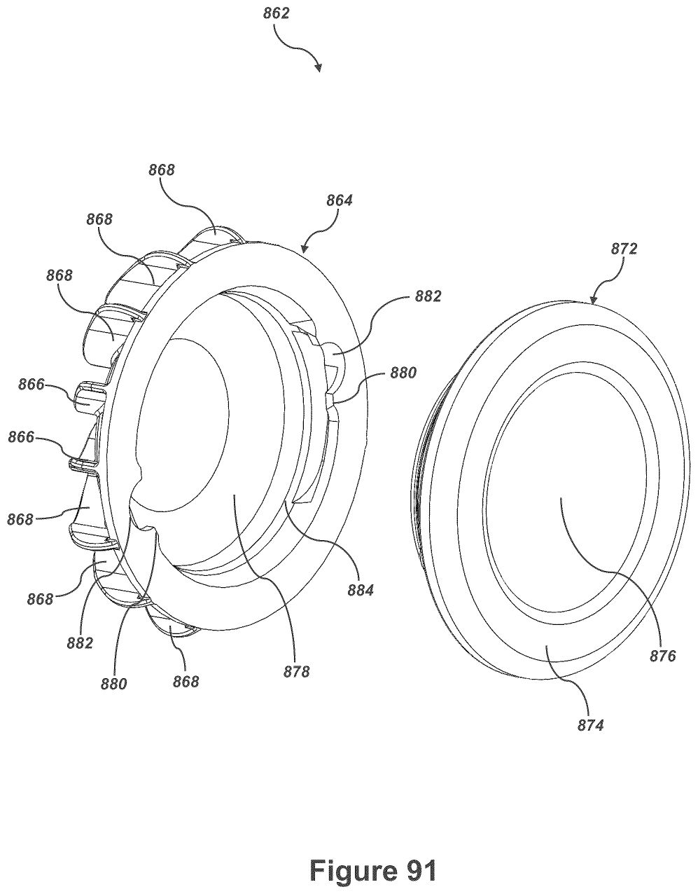

FIGS. 3-91 illustrate humeral components for use in a “reverse” type shoulder joint repair or reconstruction according to the disclosure so that the shoulder joint may be stabilized in such a way that normal joint functions can be maintained. Such components are adapted to cooperate with a ball portion of a glenoid component that is adapted for attachment to the scapula of the shoulder joint.

A first embodiment of the disclosure 122 is illustrated in FIGS. 3-6. As shown therein, stemless shell 124 includes stability fins 126 having serrated distal ends 128, a collared portion 130, and a cylindrical distal portion 132. The liner 134 includes a shoulder portion 136, and a concave portion 138 where it interfaces with the ball (not shown). Preferably, the stemless shell 124 is comprised of titanium or another biocompatible metal, and the liner 134 is comprised of a ceramic, ultrahigh molecular weight polyethylene (UHMWPE), or another suitable biocompatible material.

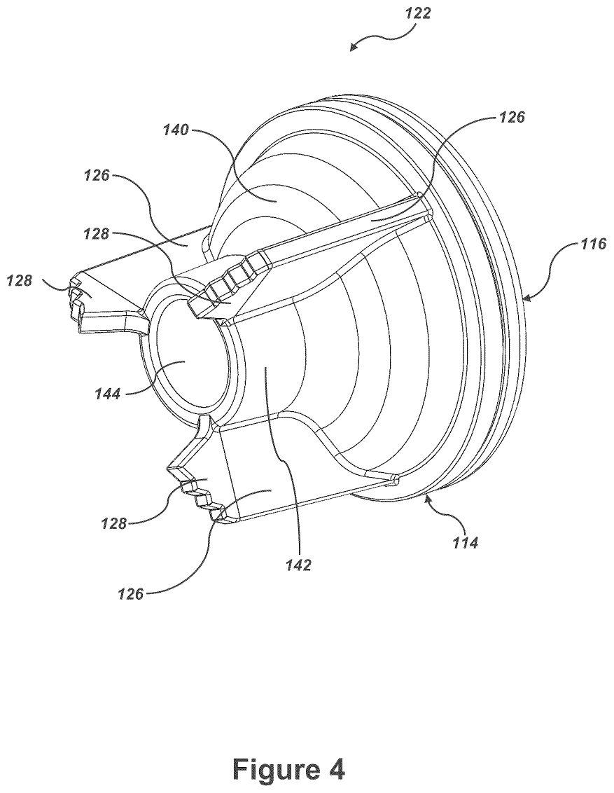

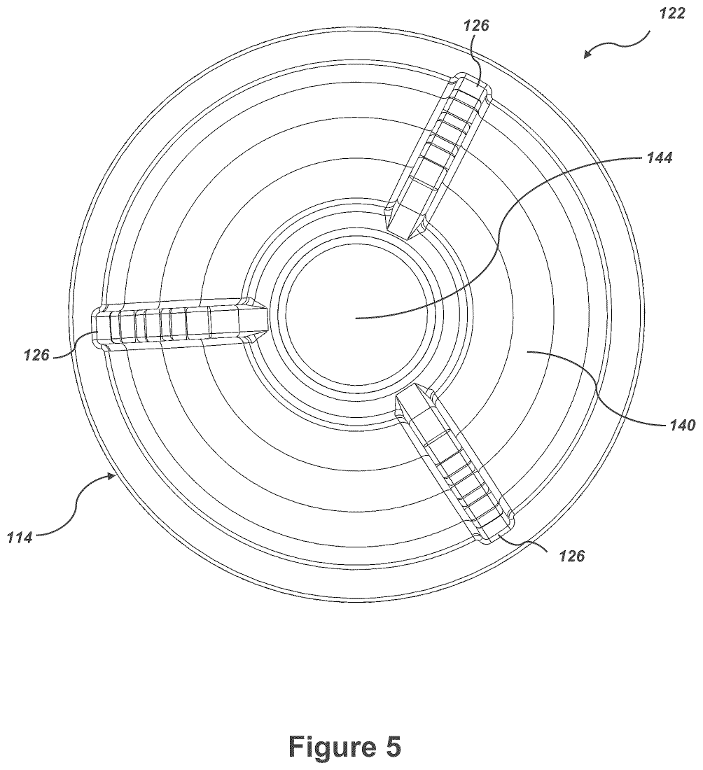

In the embodiment of the disclosure 122 illustrated in FIGS. 4 and 5, the rear portion 140 of the shell 114 is curved so as to be convex towards the stability fins 126 and can be smooth or have an irregular surface to promote bony ingrowth. In this embodiment, the distal portion 142 is circular and has a circular cavity 144 and can be smooth or have an irregular surface to promote bony ingrowth and allows for various features such as additional bony ingrowth, passage of a fastener, or provides access for easier removal during cases that may require revision.

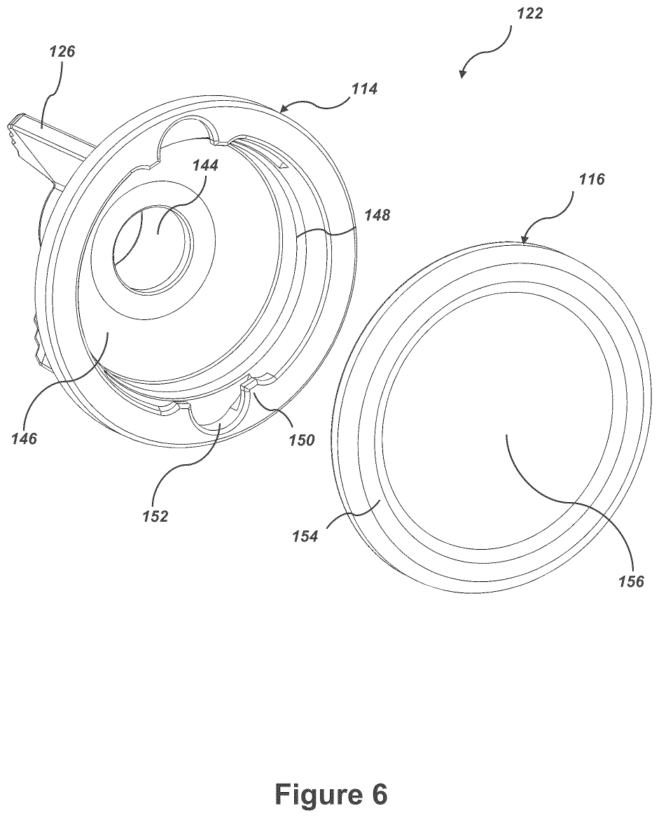

In the exploded view of the embodiment of the disclosure 122 illustrated in FIG. 6, the front cavity portion 146 has features 148, 150 and 146 which allow for attachment of liner 116. Liner 116 has a shoulder portion 154 and a concave portion 156 that interfaces with a ball (not shown).

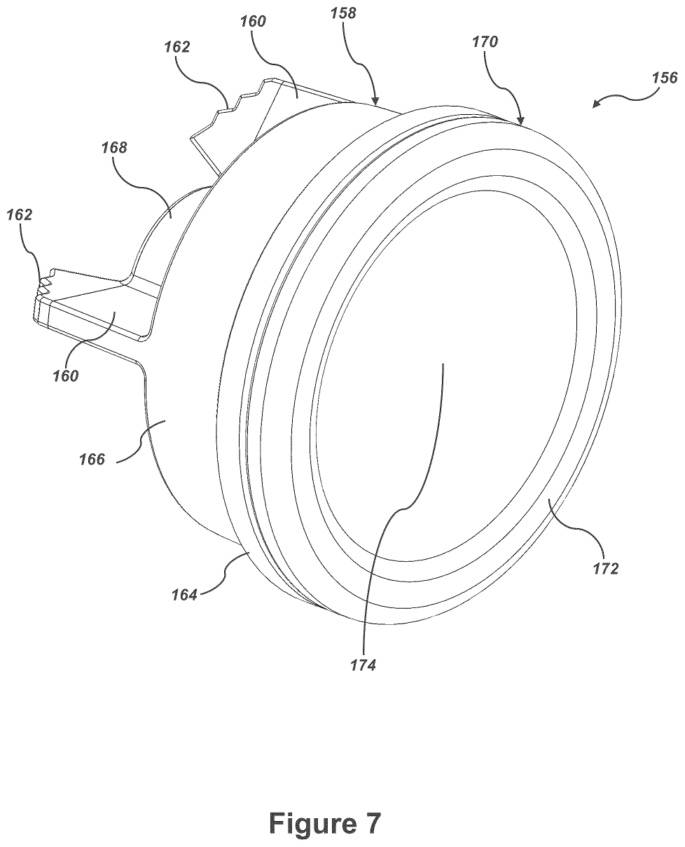

A second embodiment of the disclosure 156 is illustrated in FIGS. 7-10. As shown therein, stemless shell 158 includes stability fins 160 having serrated distal ends 162, a collared portion 164, and a proximal portion 166 and distal portion 168. The liner 170 includes a shoulder portion 172, and a concave portion 174 where it interfaces with the ball (not shown). Preferably, the stemless shell 158 is comprised of titanium or another biocompatible metal, and the liner 170 is comprised of a ceramic, ultrahigh molecular weight polyethylene (UHMWPE), or another suitable biocompatible material.

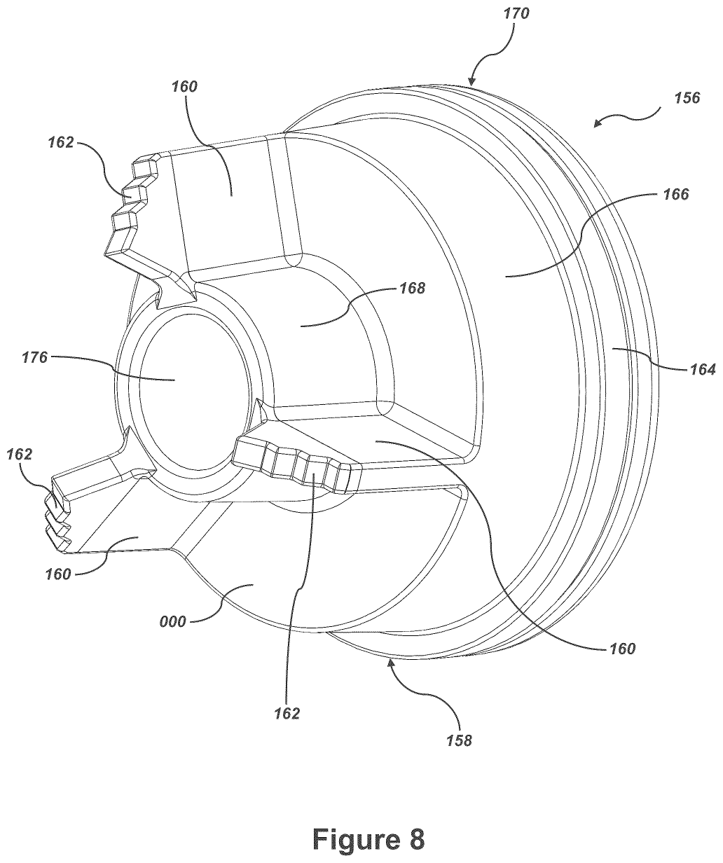

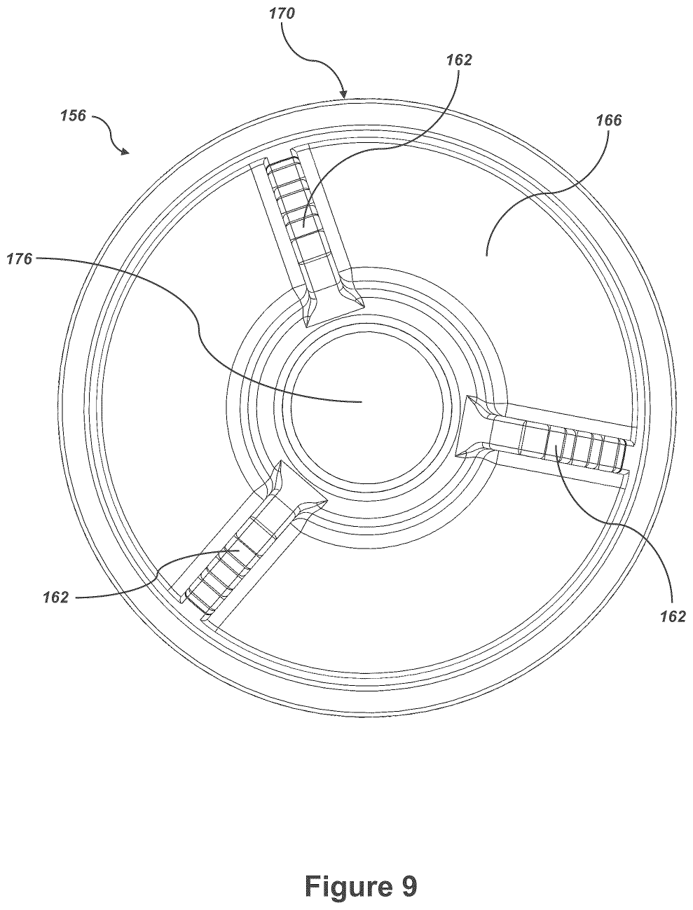

In the embodiment of the disclosure 156 illustrated in FIGS. 8 and 9, the cylindrical proximal portion 168 of the stemless shell 158 is circular with a diameter that is different than the collared portion 164 and has stability fins 160 and can be smooth or have an irregular surface to promote bony ingrowth. In this embodiment, the distal portion 168 is circular and has a circular cavity 176 and can be smooth or have an irregular surface to promote bony ingrowth and allows for various features such as additional bony ingrowth, passage of a fastener, or provides access for easier removal during cases that may require revision.

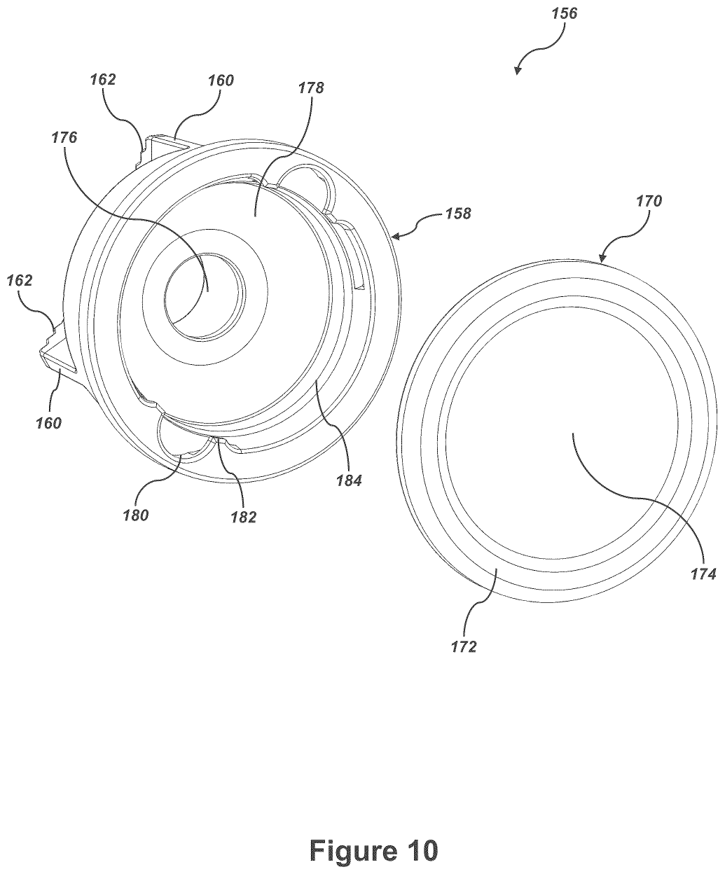

In the exploded view of the embodiment of the disclosure 156 illustrated in FIG. 10, the front cavity portion 178 has features 180, 182 and 184 which allow for attachment of liner 170. Liner 170 has a shoulder portion 172 and a concave portion 174 that interfaces with a ball (not shown).

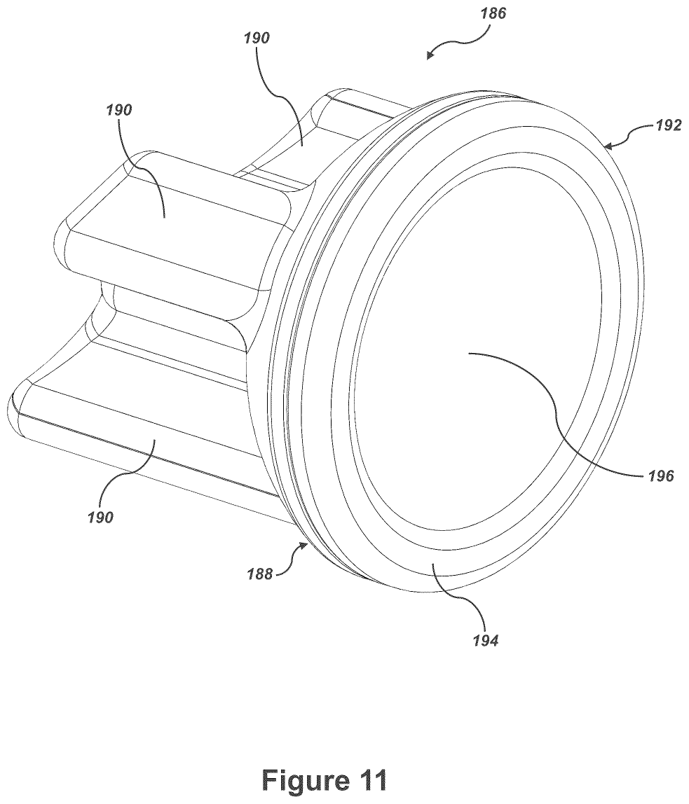

A third embodiment of the disclosure 186 is illustrated in FIGS. 11-14. As shown therein, stemless shell 188 includes varying length lobes 190. The liner 192 includes a shoulder portion 194, and a concave portion 196 where it interfaces with the ball (not shown). Preferably, the stemless shell 188 is comprised of titanium or another biocompatible metal, and the liner 192 is comprised of a ceramic, ultrahigh molecular weight polyethylene (UHMWPE), or another suitable biocompatible material.

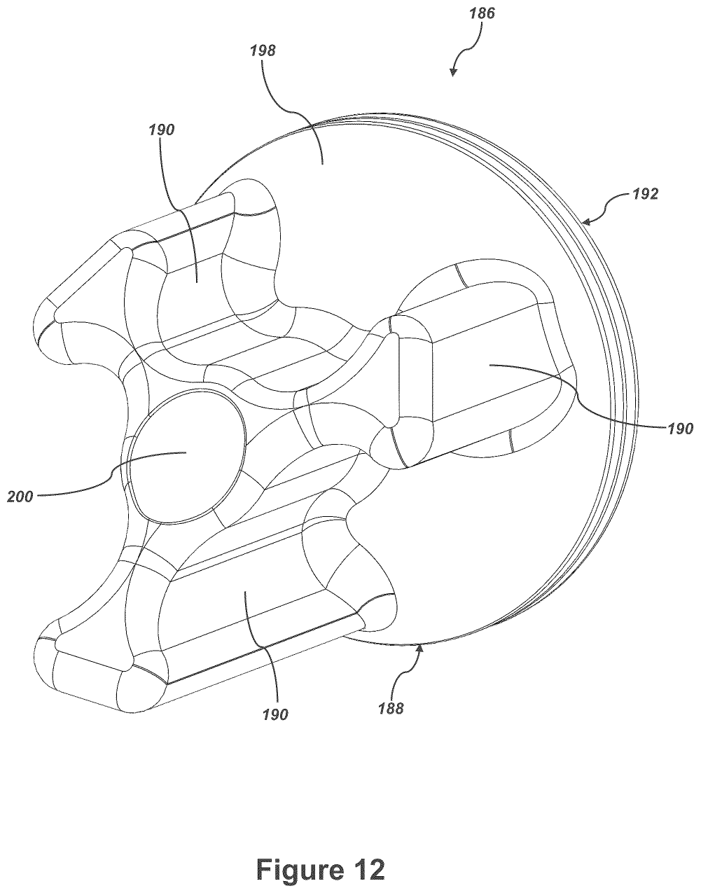

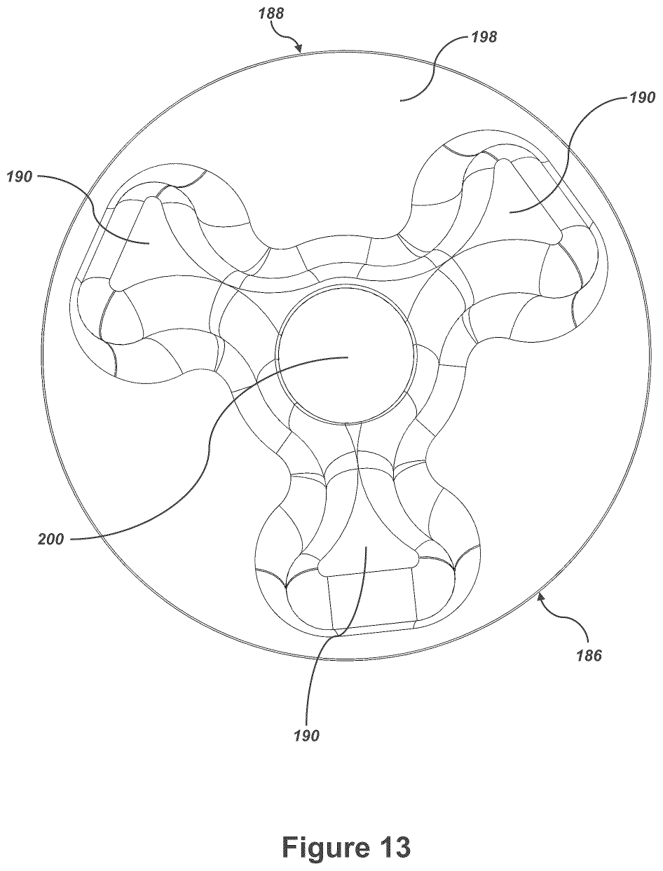

In the embodiment of the disclosure 186 illustrated in FIGS. 12 and 13, the proximal portion 198 of the stemless shell 188 is curved so as to be convex outwards towards the lobes 190 and can be smooth or have an irregular surface to promote bony ingrowth. In this embodiment, the lobes 190 are different lengths and has a circular cavity 200 and can be smooth or have an irregular surface to promote bony ingrowth and allows for various features such as additional bony ingrowth, passage of a fastener, or provides access for easier removal during cases that may require revision.

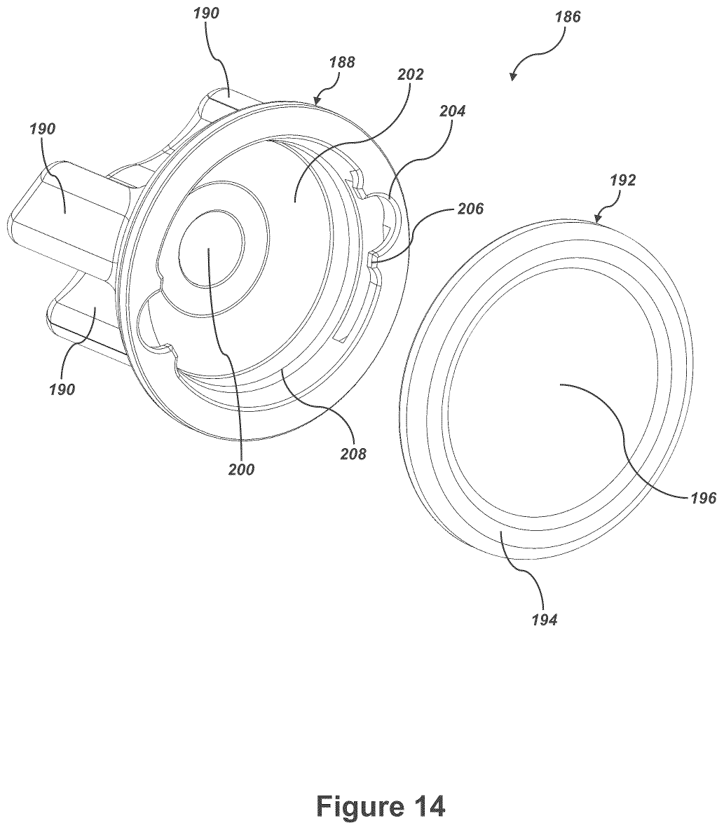

In the exploded view of the embodiment of the disclosure 186 illustrated in FIG. 14, the front cavity portion 202 has features 204, 206 and 208 which allow for attachment of liner 192. Liner 192 has a shoulder portion 194 and a concave portion 196 that interfaces with a ball (not shown).

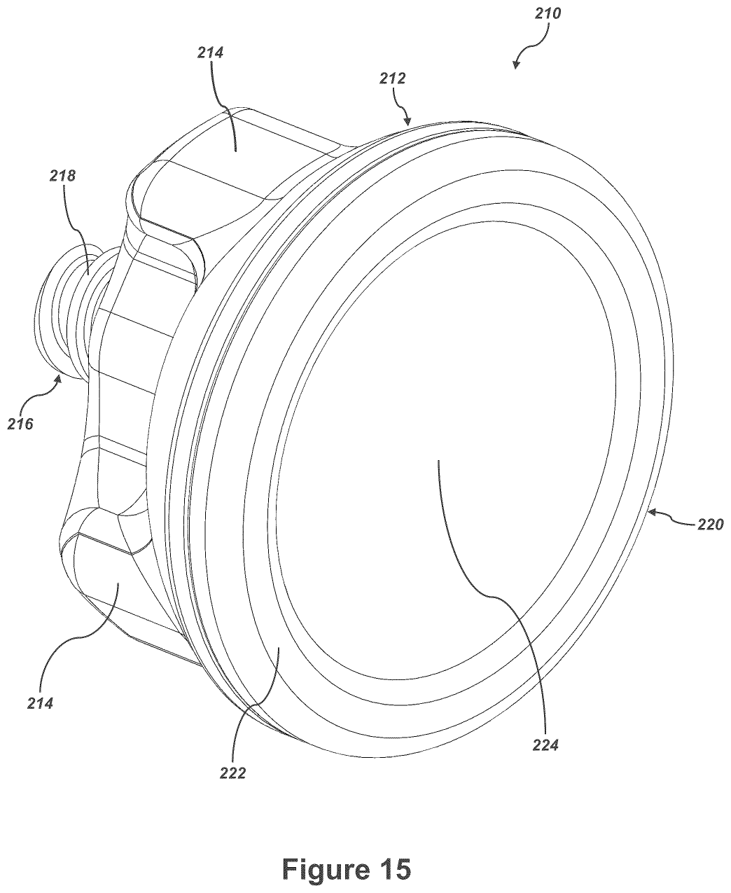

A fourth embodiment of the disclosure 210 is illustrated in FIGS. 15-18. As shown therein, stemless shell 212 includes equal length lobes 214 and a fastener 216 with threads 218. The liner 220 includes a shoulder portion 222, and a concave portion 224 where it interfaces with the ball (not shown). Preferably, the stemless shell 212 is comprised of titanium or another biocompatible metal, and the liner 220 is comprised of a ceramic, ultrahigh molecular weight polyethylene (UHMWPE), or another suitable biocompatible material.

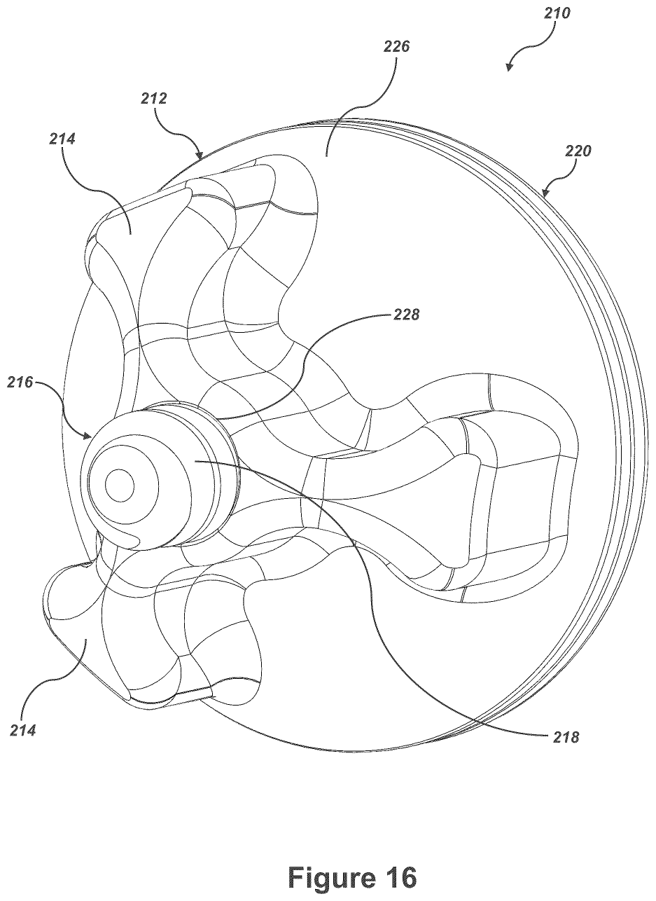

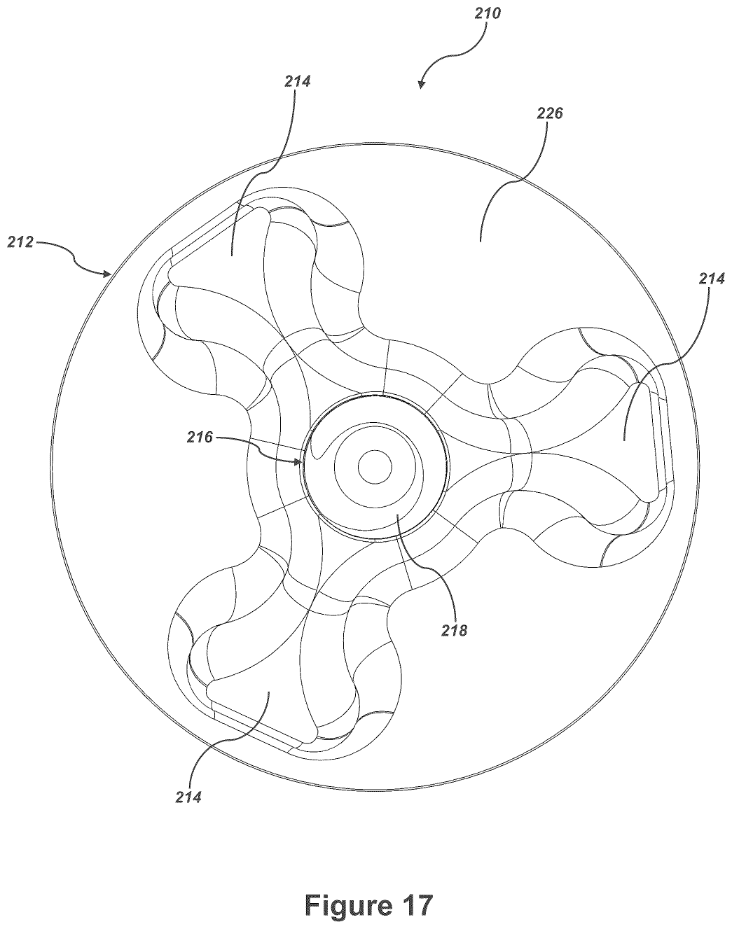

In the embodiment of the disclosure 210 illustrated in FIGS. 16 and 17, the proximal portion 226 of the stemless shell 212 is curved so as to be convex outwards towards the lobes 214 and can be smooth or have an irregular surface to promote bony ingrowth. In this embodiment, the lobes 214 are the same length and has a circular cavity 228 that accepts a fastener 216 and can be smooth or have an irregular surface to promote bony ingrowth and allows for various features such as additional bony ingrowth, passage of a fastener, or provides access for easier removal during cases that may require revision.

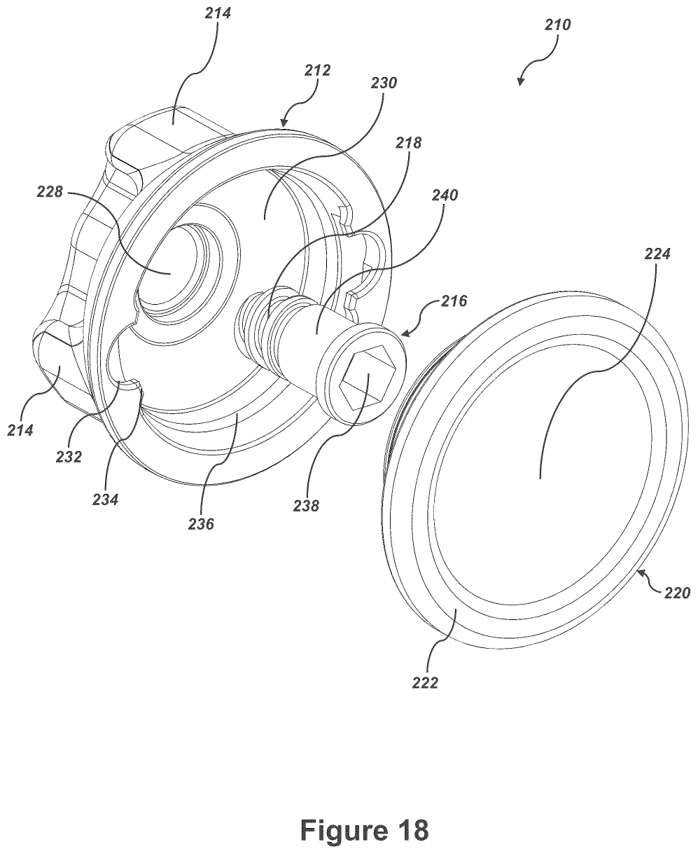

In the exploded view of the embodiment of the disclosure 210 illustrated in FIG. 18, the front cavity portion 230 has features 232, 234 and 236 which allow for attachment of liner 220. The fastener 216 includes a cavity 238 with geometry for a screwdriver, a proximal smooth section 240, and a threaded end 218. Liner 220 has a shoulder portion 222 and a concave portion 224 that interfaces with a ball (not shown).

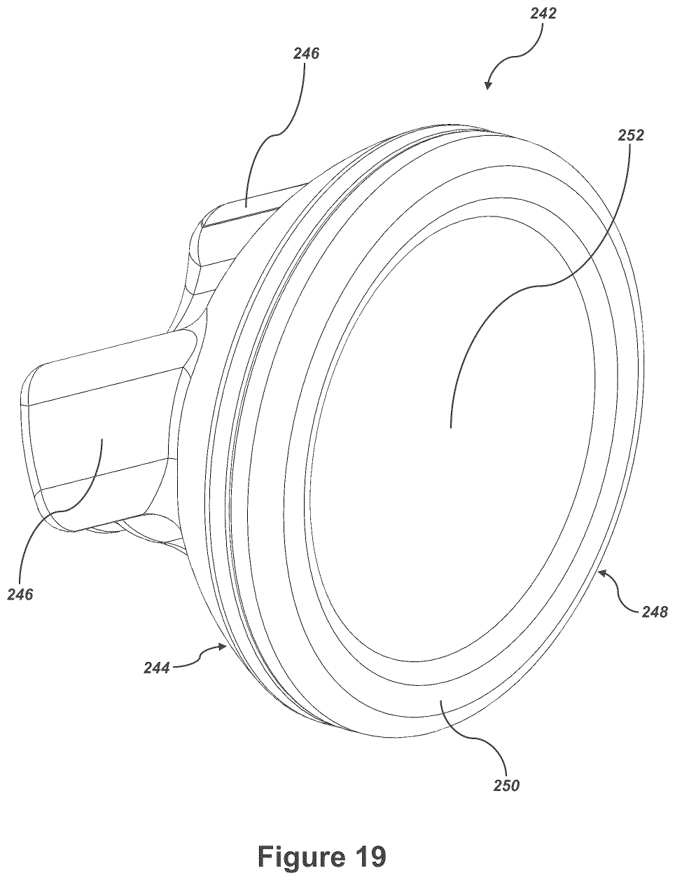

A fifth embodiment of the disclosure 242 is illustrated in FIGS. 19-23. As shown therein, stemless shell 244 includes angled, varying length lobes 246. The liner 248 includes a shoulder portion 250, and a concave portion 252 where it interfaces with the ball (not shown). Preferably, the stemless shell 244 is comprised of titanium or another biocompatible metal, and the liner 248 is comprised of a ceramic, ultrahigh molecular weight polyethylene (UHMWPE), or another suitable biocompatible material.

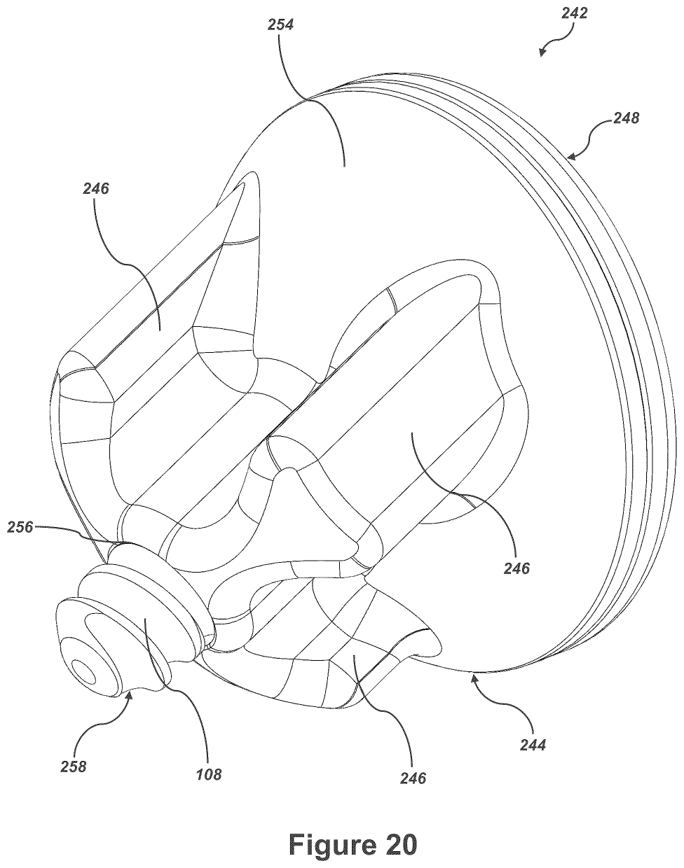

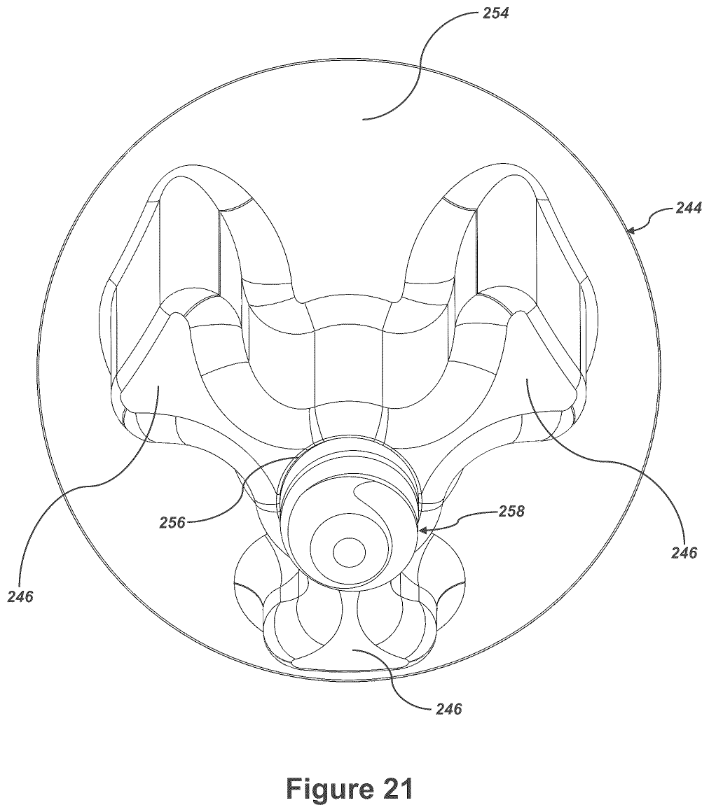

In the embodiment of the disclosure 242 illustrated in FIGS. 20 and 21, the proximal portion 254 of the stemless shell 244 is curved so as to be convex outwards towards the lobes 246 and can be smooth or have an irregular surface to promote bony ingrowth. In this embodiment, the lobes 246 are the same length and angled inferiorly, and has a circular cavity 256 that accepts a fastener 258 and can be smooth or have an irregular surface to promote bony ingrowth and allows for various features such as additional bony ingrowth, passage of a fastener, or provides access for easier removal during cases that may require revision.

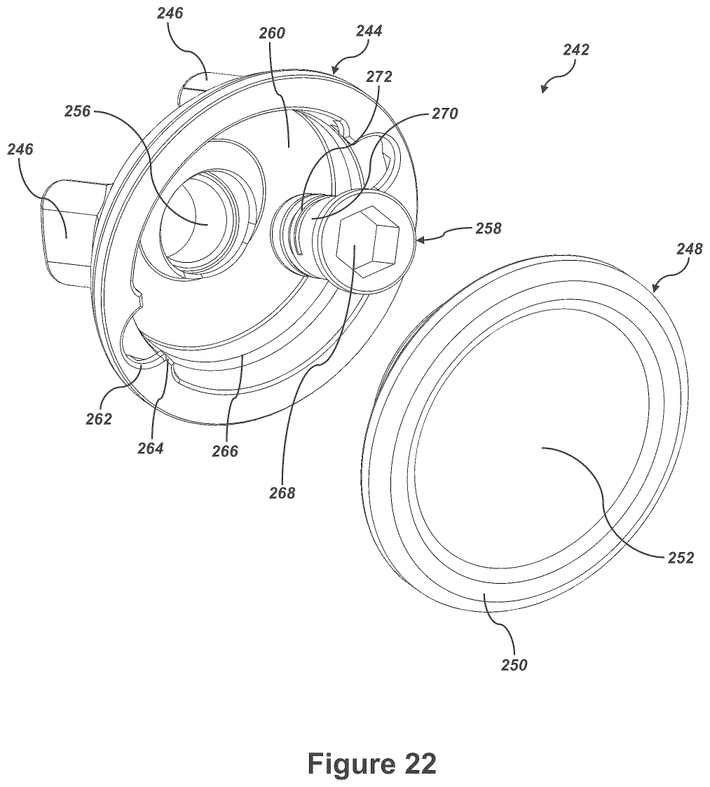

In the exploded view of the embodiment of the disclosure 242 illustrated in FIG. 22, the front cavity portion 260 has features 262, 264 and 266 which allow for attachment of liner 248. The fastener 258 includes a cavity 256 with geometry for a screwdriver 268, a proximal smooth section 270, and a threaded end 272. Liner 248 has a shoulder portion 250 and a concave portion 224 that interfaces with a ball (not shown).

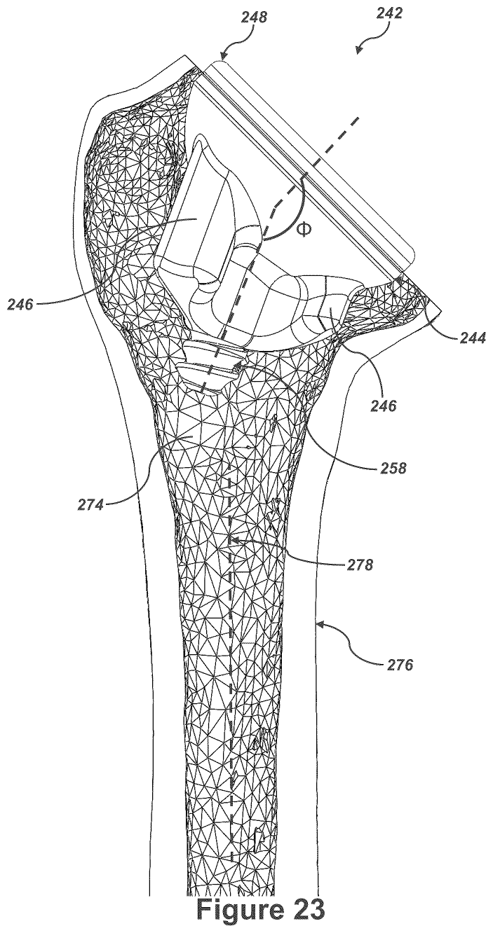

In the section view of the disclosure 242 illustrated in FIG. 23, the stemless shell 244 has lobes 246 which are the same length and tilted inferiorly by angle (into the humeral shaft 272 of the humerus 276. Angle (can range from 1800 to an angle that follows a line 278 down the central canal of the humerus.

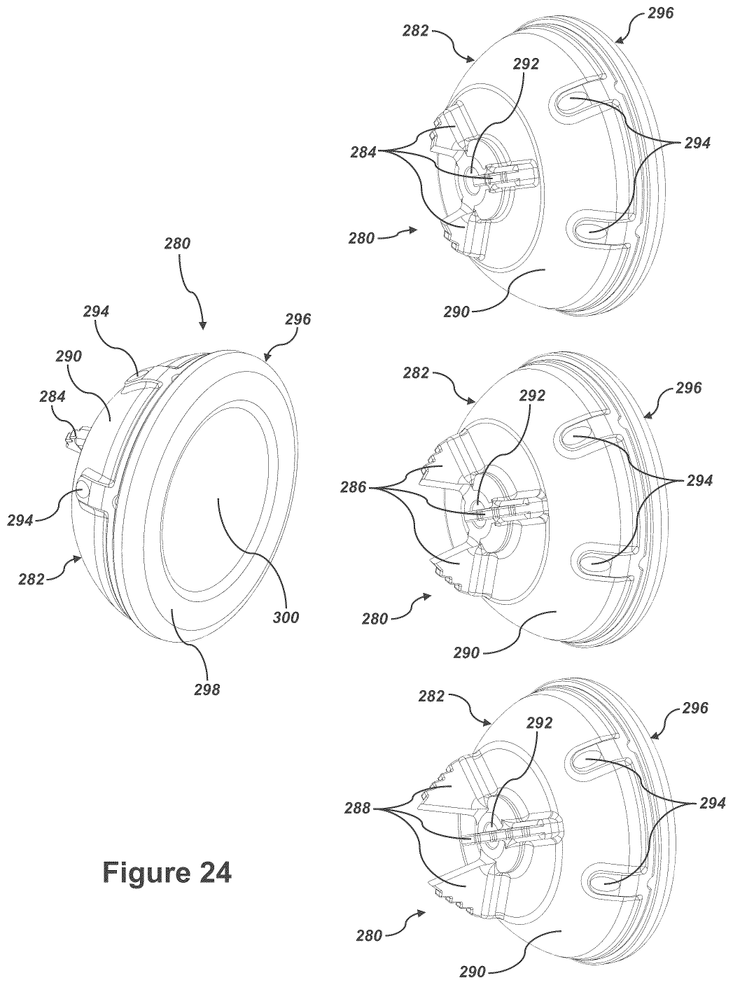

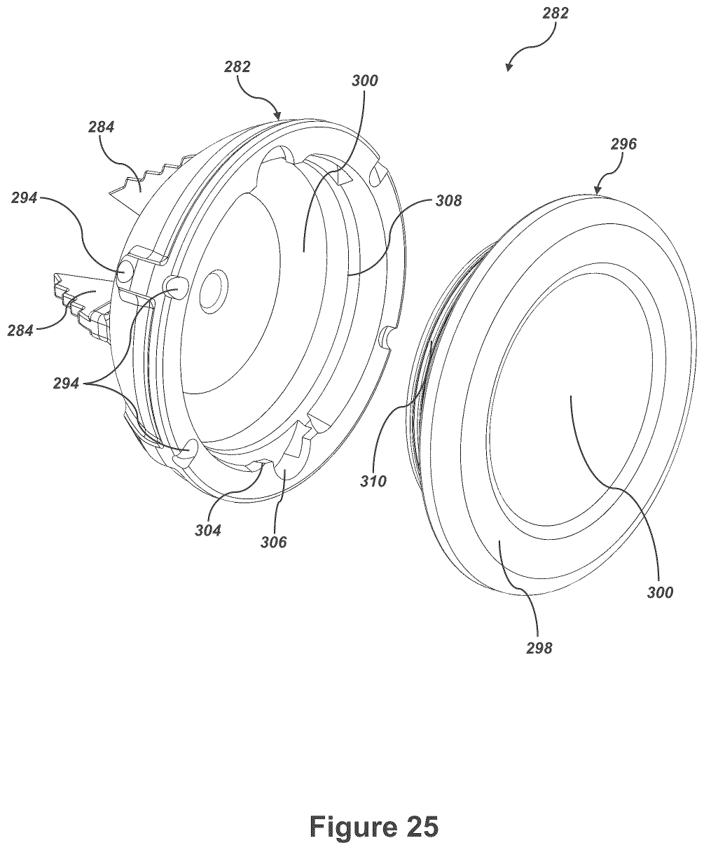

A sixth embodiment of the disclosure 280 is illustrated in FIGS. 24-25. As shown therein, a stemless shell 282 includes stability fins in differing lengths; small 284, medium 286, and large 288, a convex portion 290, a circular cavity 292 which can be smooth or have an irregular surface to promote bony ingrowth and allows for various features such as additional bony ingrowth, passage of a fastener, or provides access for easier removal during cases that may require revision, and features for passing sutures 294. The liner 296 includes a shoulder portion 298, and a concave portion 300 where it interfaces with the ball (not shown). Preferably, the stemless shell 282 is comprised of titanium or another biocompatible metal, and the liner 296 is comprised of a ceramic, ultrahigh molecular weight polyethylene (UHMWPE), or another suitable biocompatible material.

In the exploded view of the embodiment of the disclosure 280 illustrated in FIG. 25, the front cavity portion 302 has features 304, 306 and 308 which allow for attachment of liner 296. Liner 296 has a shoulder portion 298, grooved teeth 310 for attachment to liner 296, and a concave portion 302 that interfaces with a ball (not shown).

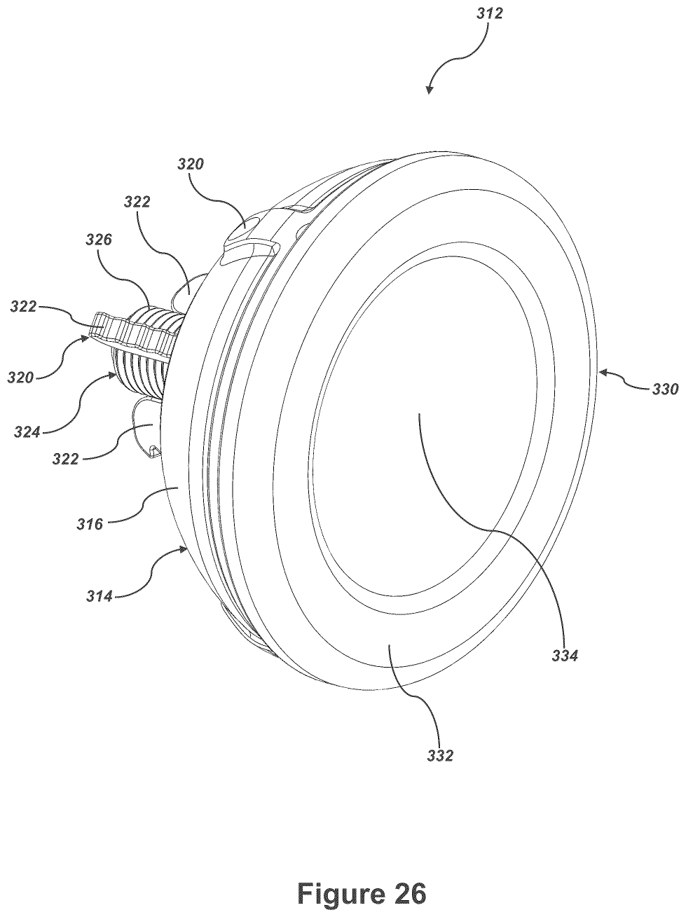

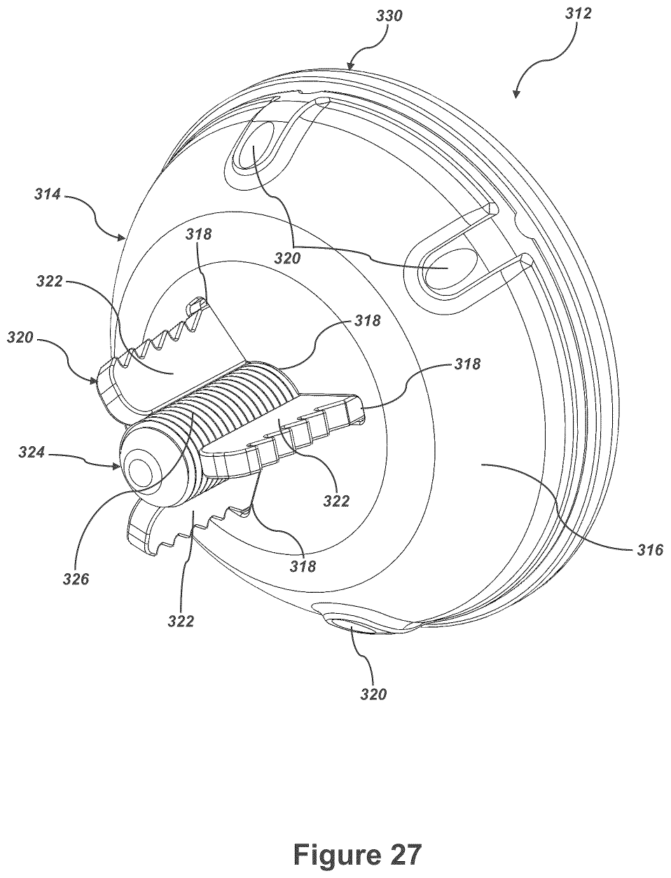

A seventh embodiment of the disclosure 312 is illustrated in FIGS. 26-28. As shown therein, a stemless shell 314 including a convex portion 316, an irregularly shaped opening 318 which can accept an insert 320 which has flexible serrated fins 322 that can be deployed outwardly by the action of threads 326 on a fastener 324 with a tapered end 328, and features for passing sutures 320. Insert 320 can be smooth or have an irregular surface to promote bony ingrowth and can be made of plastic, metal, or another suitable biocompatible material. The liner 330 includes a shoulder portion 332, and a concave portion 334 where it interfaces with the ball (not shown). Preferably, the stemless shell 314 is comprised of titanium or another biocompatible metal, and the liner 330 is comprised of a ceramic, ultrahigh molecular weight polyethylene (UHMWPE), or another suitable biocompatible material.

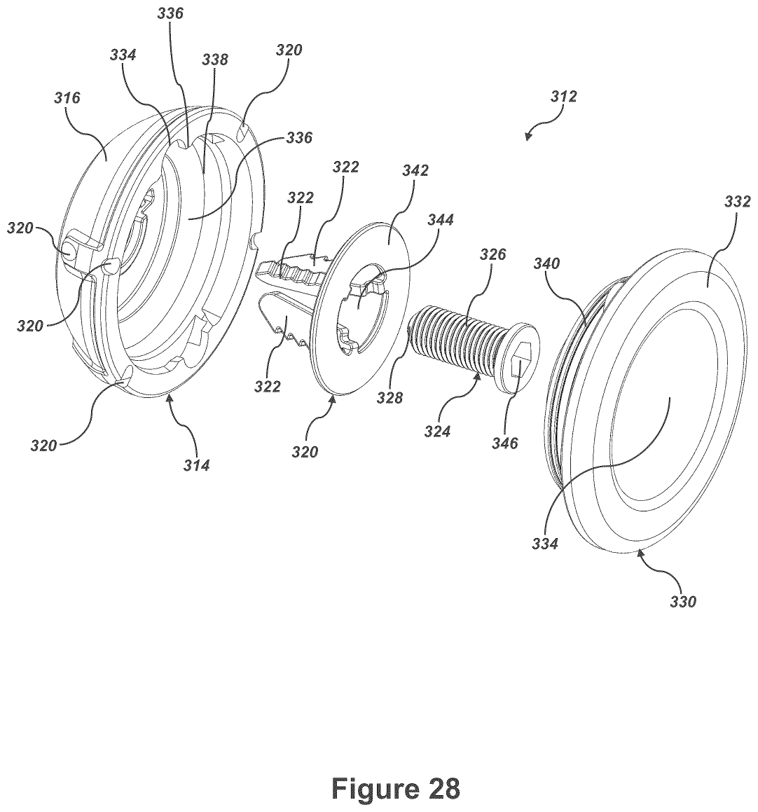

In the exploded view of the embodiment of the disclosure 312 illustrated in FIG. 28, the front cavity portion 336 has features 334, 336 and 338 which allow for attachment of liner 330. Liner 330 has a shoulder portion 332, grooved teeth 340 for attachment to liner 330, and a concave portion 334 that interfaces with a ball (not shown). Insert 320 (shown in its undeployed state in FIG. 28) has a ring structure 342 that has an aperture 344 that accepts fastener 324 driven using geometry 346 to accept a screwdriver.

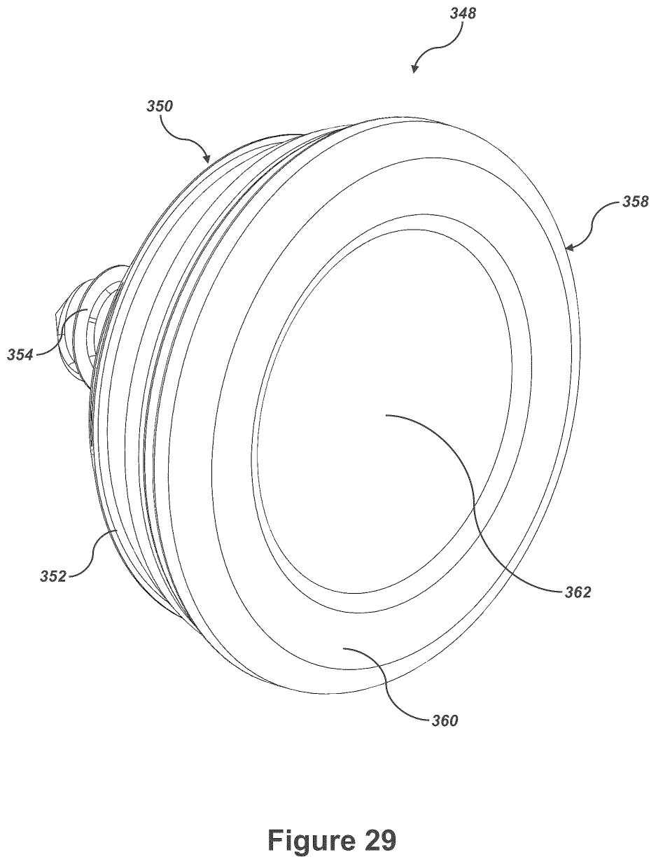

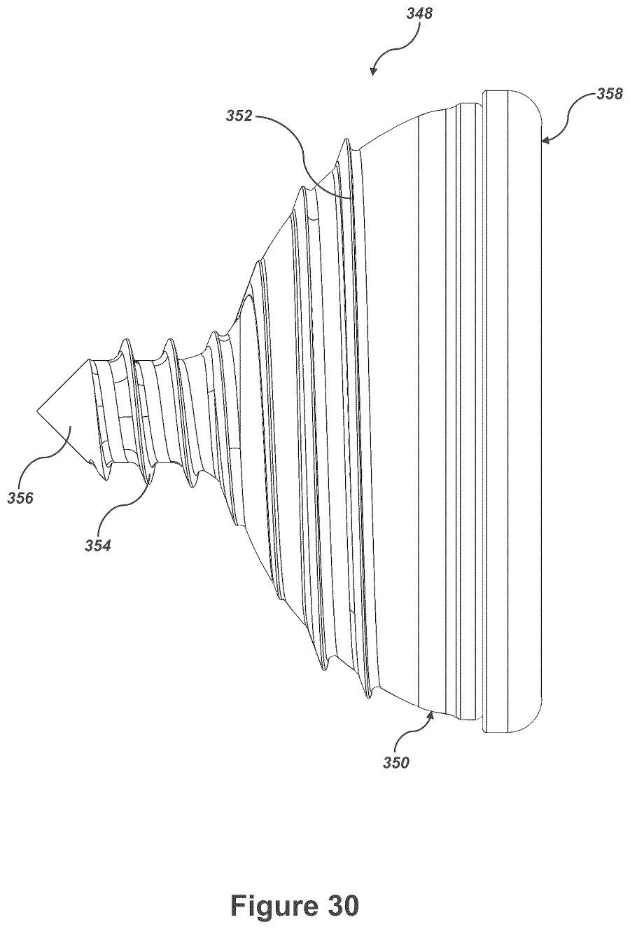

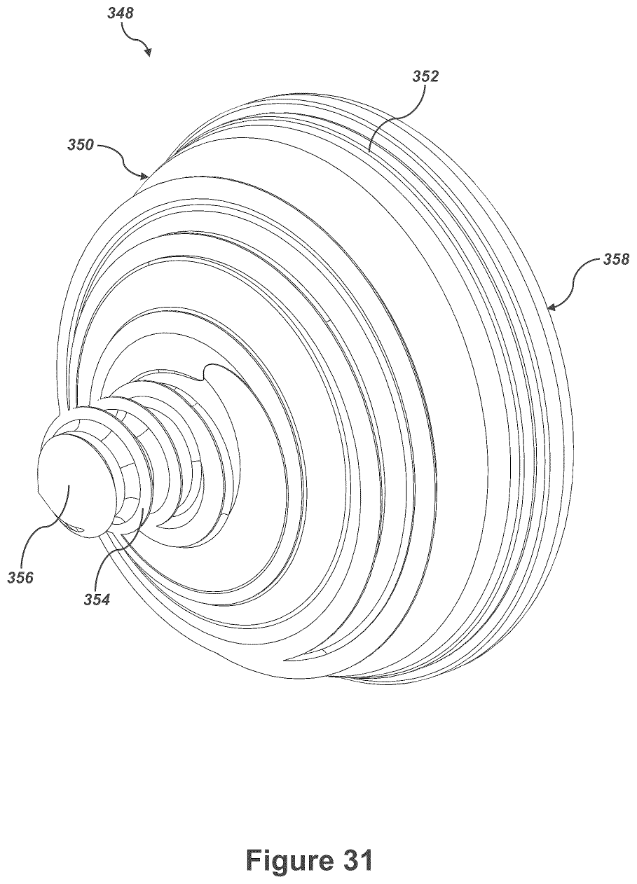

An eighth embodiment of the disclosure 348 is illustrated in FIGS. 29-32. As shown therein, a stemless shell 350 including a proximally threaded portion 352 and a smaller diameter distally threaded portion 354 with a tapered tip 356. The liner 358 includes a shoulder portion 360, and a concave portion 362 where it interfaces with the ball (not shown). Preferably, the stemless shell 350 is comprised of titanium or another biocompatible metal, and the liner 358 is comprised of a ceramic, ultrahigh molecular weight polyethylene (UHMWPE), or another suitable biocompatible material.

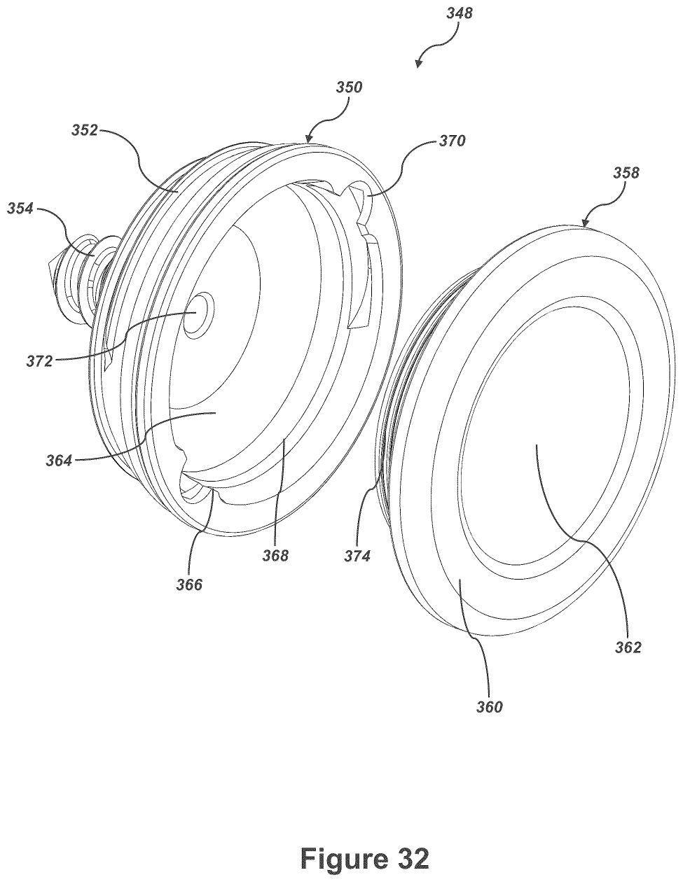

In the exploded view of the embodiment of the disclosure 348 illustrated in FIG. 32, the front cavity portion 364 has features 366, 368 and 370 which allow for attachment of liner 358 and a threaded hole 372 for a fastener. Liner 358 has a shoulder portion 360, grooved teeth 374 for attachment to liner 358, and a concave portion 362 that interfaces with a ball (not shown).

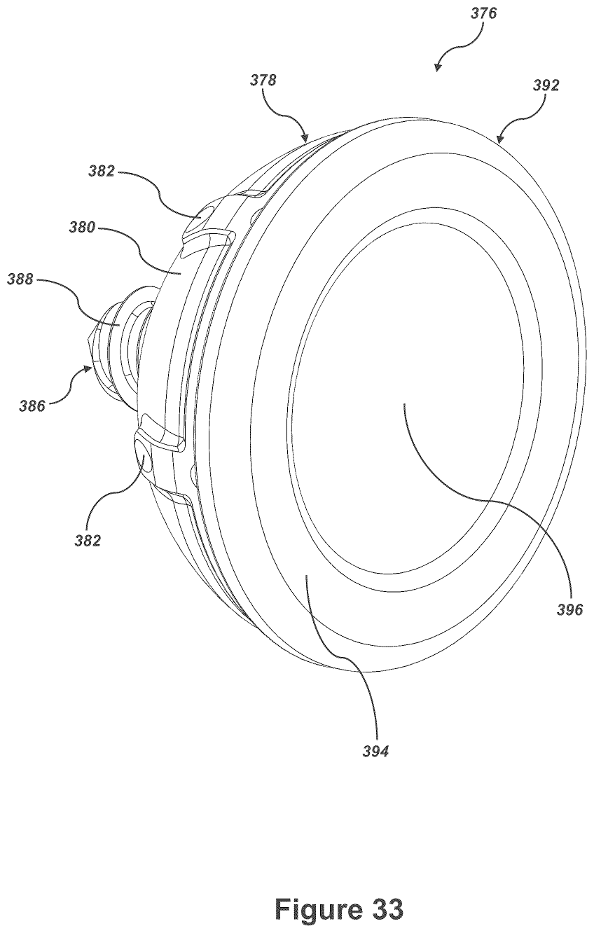

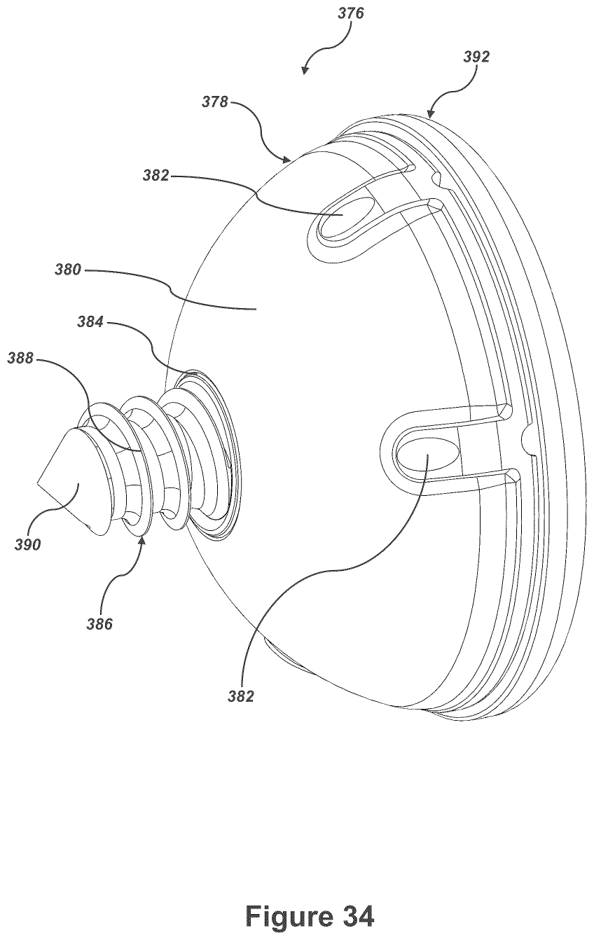

A ninth embodiment of the disclosure 376 is illustrated in FIGS. 33-35. As shown therein, a stemless shell 378 including a convex portion 380, features for passing sutures 382, and an aperture 384 that accepts a fastener 386 with large threads 388 and a tapered end 390. The liner 392 includes a shoulder portion 394, and a concave portion 396 where it interfaces with the ball (not shown). Preferably, the stemless shell 378 is comprised of titanium or another biocompatible metal, and the liner 396 is comprised of a ceramic, ultrahigh molecular weight polyethylene (UHMWPE), or another suitable biocompatible material.

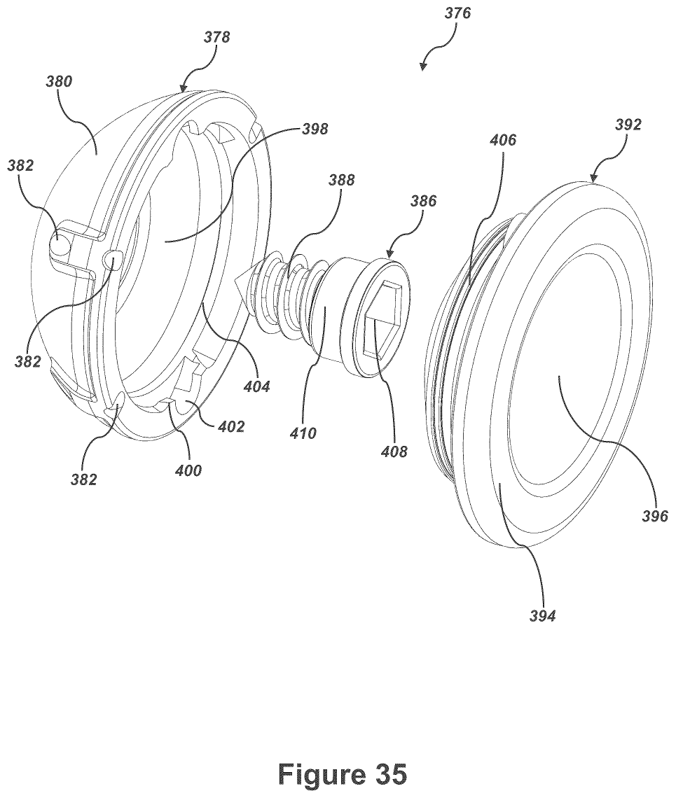

In the exploded view of the embodiment of the disclosure 376 illustrated in FIG. 35, the front cavity portion 398 has features 400, 402 and 404 which allow for attachment of liner 392. Liner 392 has a shoulder portion 394, grooved teeth 406 for attachment to liner 392, and a concave portion 396 that interfaces with a ball (not shown). Fastener 386 has a geometry 408 to accept a screwdriver and is smooth proximally 410.

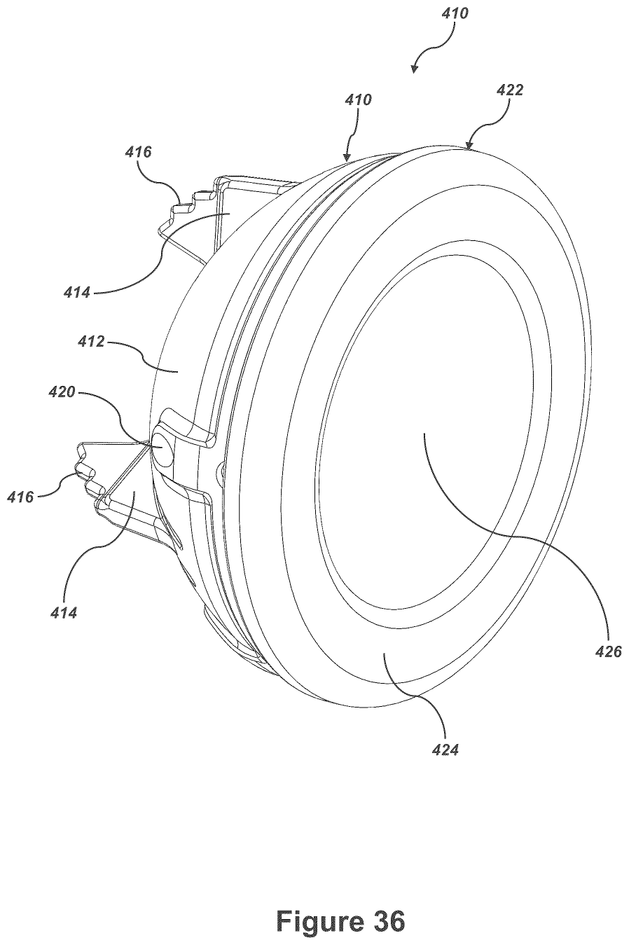

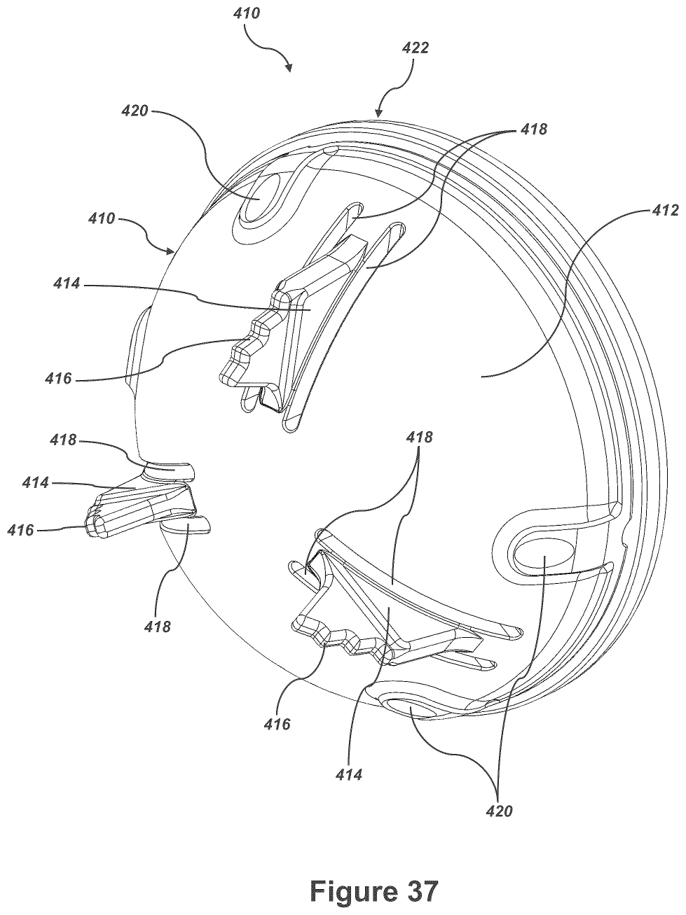

A tenth embodiment of the disclosure 408 is illustrated in FIGS. 36-38. As shown therein, a stemless shell 410 including a convex portion 412, stability fins 414 having serrated distal ends 416, slots/openings 418 which can accept various devices for cutting bone, such as a saw blade, burr, or other suitable cutting instrument, and features for passing various types of sutures 420. The liner 422 includes a shoulder portion 424, and a concave portion 426 where it interfaces with the ball (not shown). Preferably, the stemless shell 410 is comprised of titanium or another biocompatible metal, and the liner 422 is comprised of a ceramic, ultrahigh molecular weight polyethylene (UHMWPE), or another suitable biocompatible material.

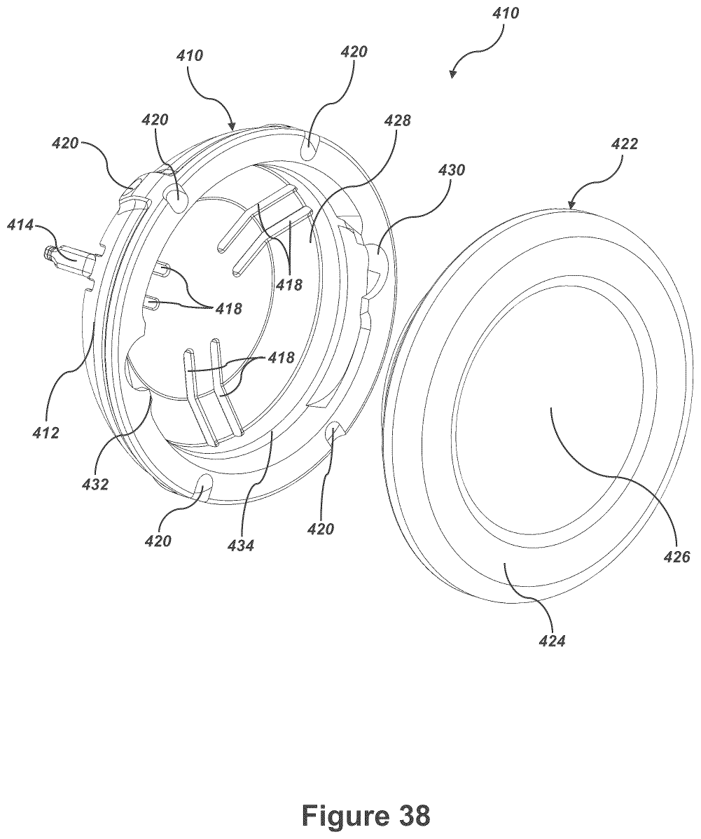

In the exploded view of the embodiment of the disclosure 408 illustrated in FIG. 38, the front cavity portion 428 has features 430, 432 and 434 which allow for attachment of liner 408. Liner 408 has a shoulder portion 424, and a concave portion 426 that interfaces with a ball (not shown).

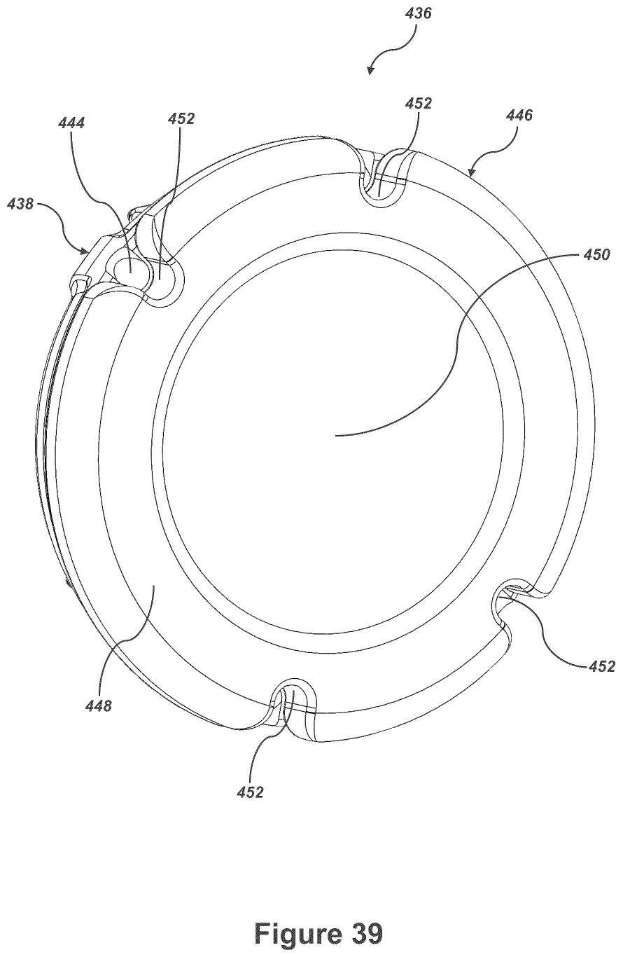

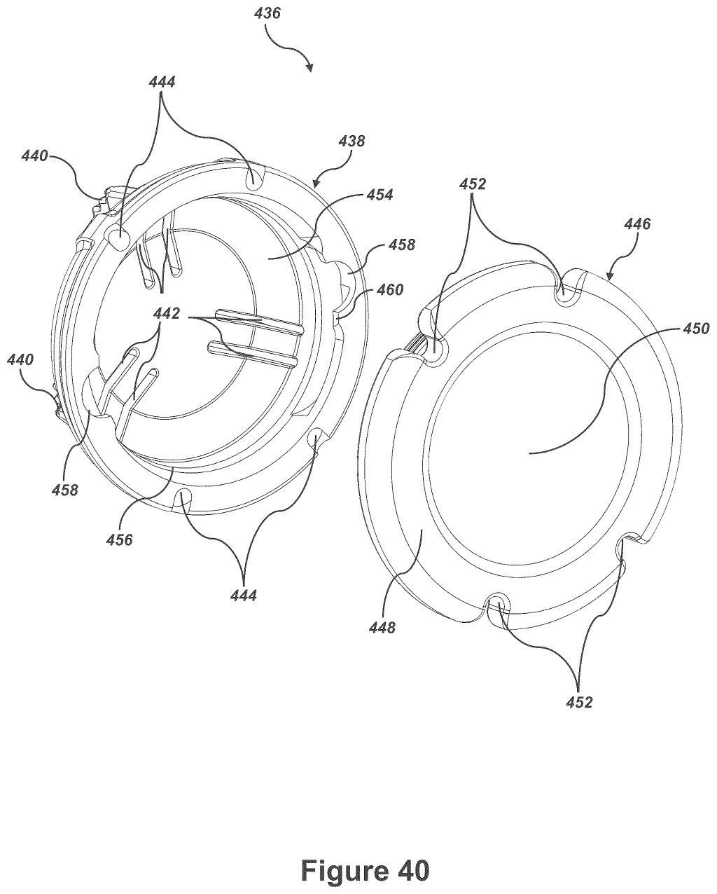

An eleventh embodiment of the disclosure 436 is illustrated in FIGS. 39-40. As shown therein, a stemless shell 438, stability fins 440 with serrated teeth on their ends, slots/openings 442 which can accept various devices for cutting bone, such as a saw blade, burr, or other suitable cutting instrument, and features 444 for passing various types of sutures or attachment sites for a device to apply compression to liner 446. The liner 446 includes a shoulder portion 448, a concave portion 450 where it interfaces with the ball (not shown), and openings 452 shaped to allow for passage of sutures or a device to apply compression to liner 446. Preferably, the stemless shell 438 is comprised of titanium or another biocompatible metal, and the liner 446 is comprised of a ceramic, ultrahigh molecular weight polyethylene (UHMWPE), or another suitable biocompatible material.

In the exploded view of the embodiment of the disclosure 436 illustrated in FIG. 40, the front cavity portion 454 has features 456, 458 and 460 which allow for attachment of liner 446. Liner 446 has a shoulder portion 448, openings 452, and a concave portion 450 that interfaces with a ball (not shown).

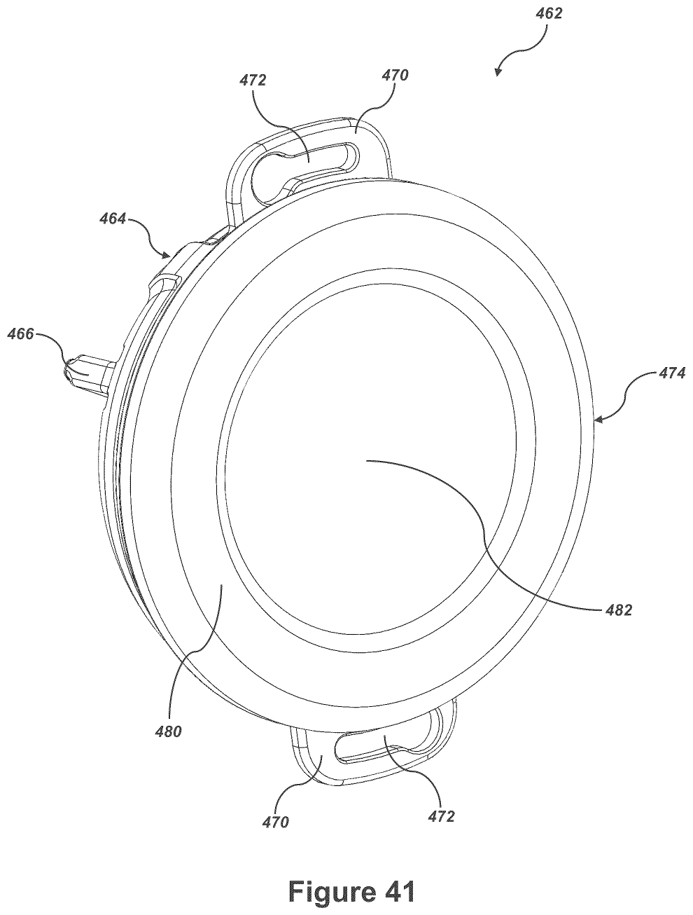

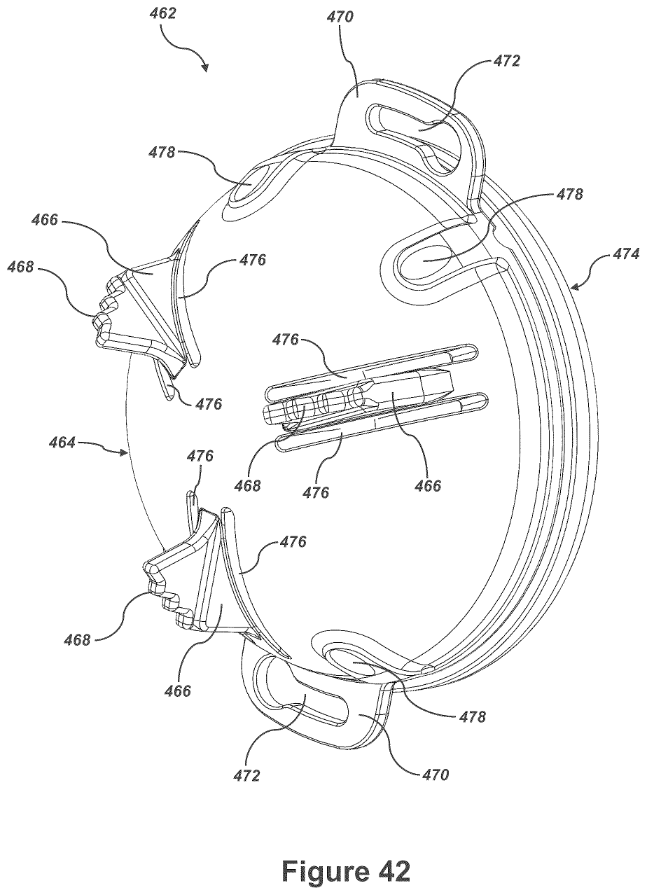

A twelfth embodiment of the disclosure 462 is illustrated in FIGS. 41-43. As shown therein, a stemless shell 464, stability fins 466 with serrated teeth 468 on their ends, tabbed portions 470 with slots/openings 472 for attachment of a device to apply compression to liner 474, slots/openings 476 which can accept various devices for cutting bone, such as a saw blade, burr, or other suitable cutting instrument, and features 478 for passing various types of sutures or attachment sites for a device to apply compression to liner 474. The liner 474 includes a shoulder portion 480, and a concave portion 482. Preferably, the stemless shell 464 is comprised of titanium or another biocompatible metal, and the liner 474 is comprised of a ceramic, ultrahigh molecular weight polyethylene (UHMWPE), or another suitable biocompatible material.

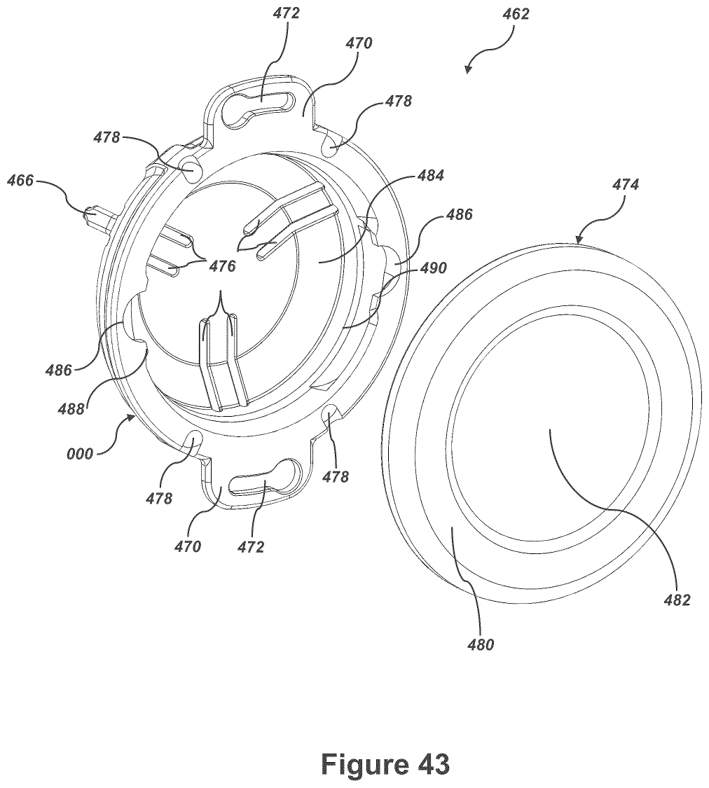

In the exploded view of the embodiment of the disclosure 462 illustrated in FIG. 43, the front cavity portion 484 has features 486, 488 and 490 which allow for attachment of liner 474. Liner 474 has a shoulder portion 480, and a concave portion 482 that interfaces with a ball (not shown).

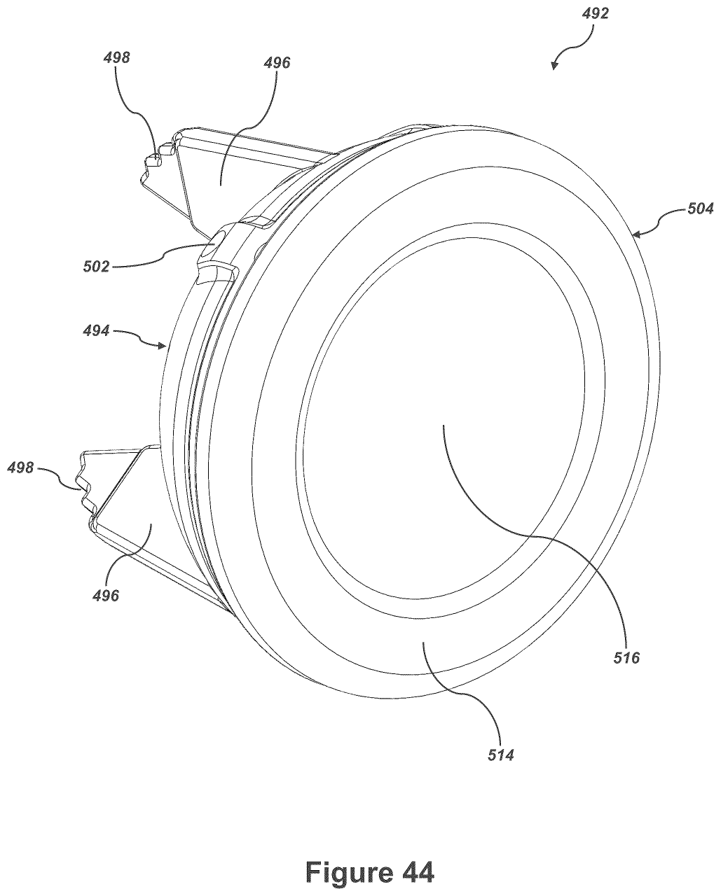

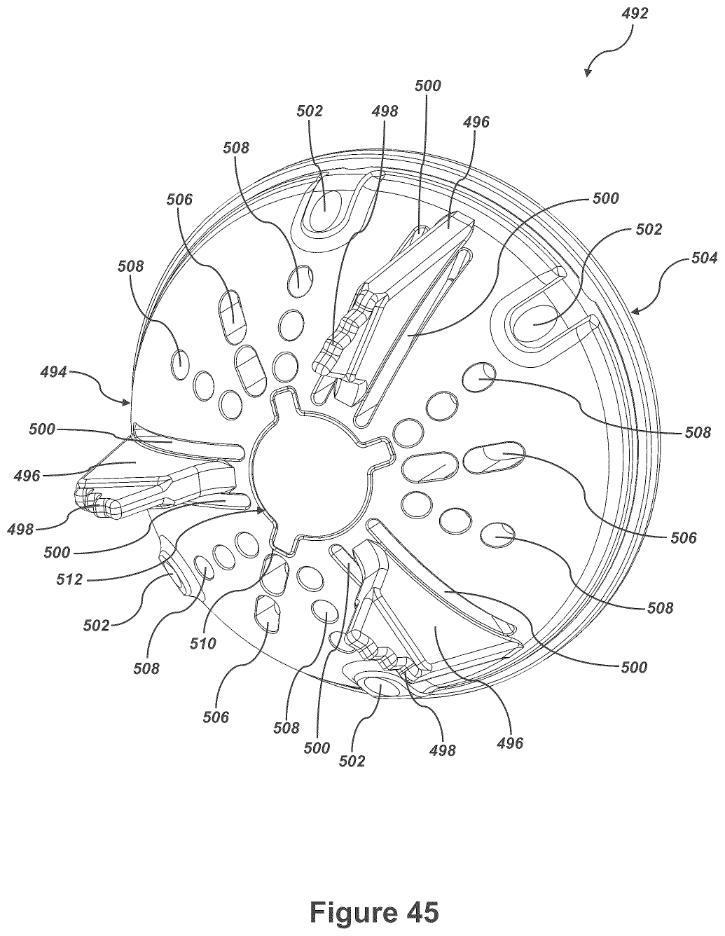

A thirteenth embodiment of the disclosure 492 is illustrated in FIGS. 44-49. As shown therein, a stemless shell 494, stability fins 496 with serrated teeth 498 on their ends, slots/openings 500 which can accept various devices for cutting bone, such as a saw blade, burr, or other suitable cutting instrument, features 502 for passing various types of sutures or attachment sites for a device to apply compression to liner 504, features 506 to apply suction, features 508 to administer bone cement, allograft, bone substitute, or another suitable biocompatible material, and a cavity 510 can accept insert 512, stem or another biocompatible device. The liner 504 includes a shoulder portion 514, and a concave portion 516. Preferably, the stemless shell 494 is comprised of titanium or another biocompatible metal, and the liner 504 is comprised of a ceramic, ultrahigh molecular weight polyethylene (UHMWPE), or another suitable biocompatible material.

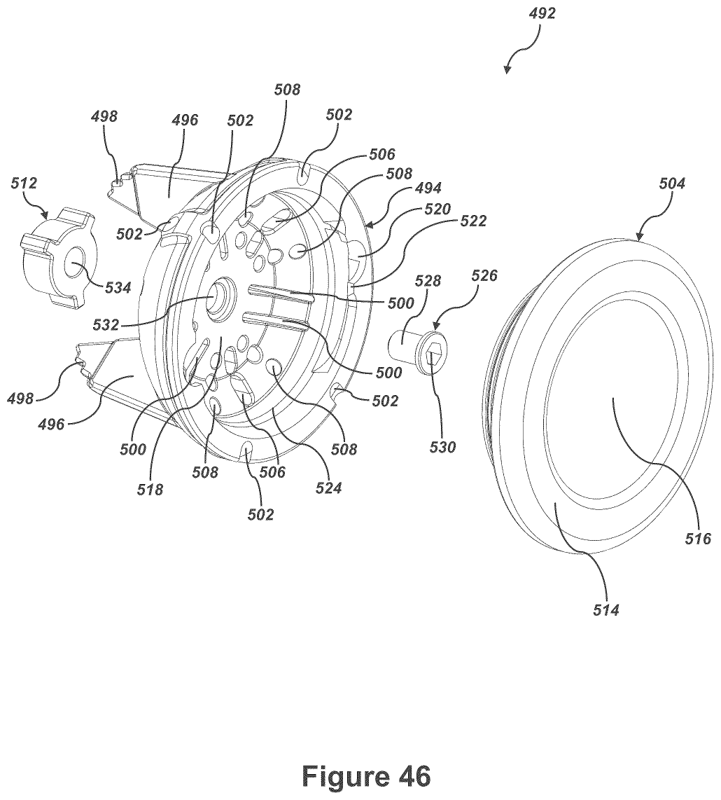

In the exploded view of the embodiment of the disclosure 492 illustrated in FIG. 46, the front cavity portion 518 has features 520, 522 and 524 which allow for attachment of liner 504. Insert 512 can be attached using a tapered junction or by a fastener 526 with threads 528 and geometry 530 to attach to a screwdriver. Also shown is the stemless shell 494, stability fins 496 with serrated teeth 498 on their ends, slots/openings 500 which can accept various devices for cutting bone, such as a saw blade, burr, or other suitable cutting instrument, features 502 for passing various types of sutures or attachment sites for a device to apply compression to liner 504, features 506 to apply suction, features 508 to administer bone cement, allograft, bone substitute, or another suitable biocompatible material, and a cavity 532 can accept fastener 526, with said fastener attaching to threaded hole 534 in insert 512. Liner 504 has a shoulder portion 514, and a concave portion 516 that interfaces with a ball (not shown).

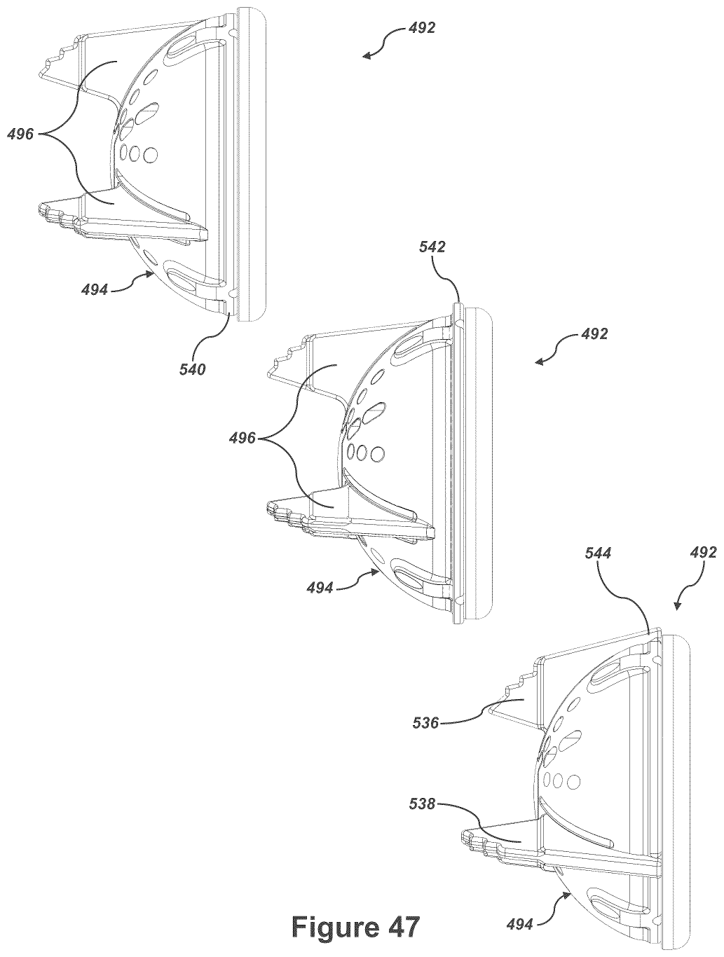

In the views of the embodiment of the disclosure 492 illustrated in FIG. 47, the stability fins can be of the same length 496 or can be of different lengths 536 and 538. The stemless shell 494 can have a flush collar 540, a larger diameter collar 542 or can have the stability fins extend past the edge of the stemless shell 544.

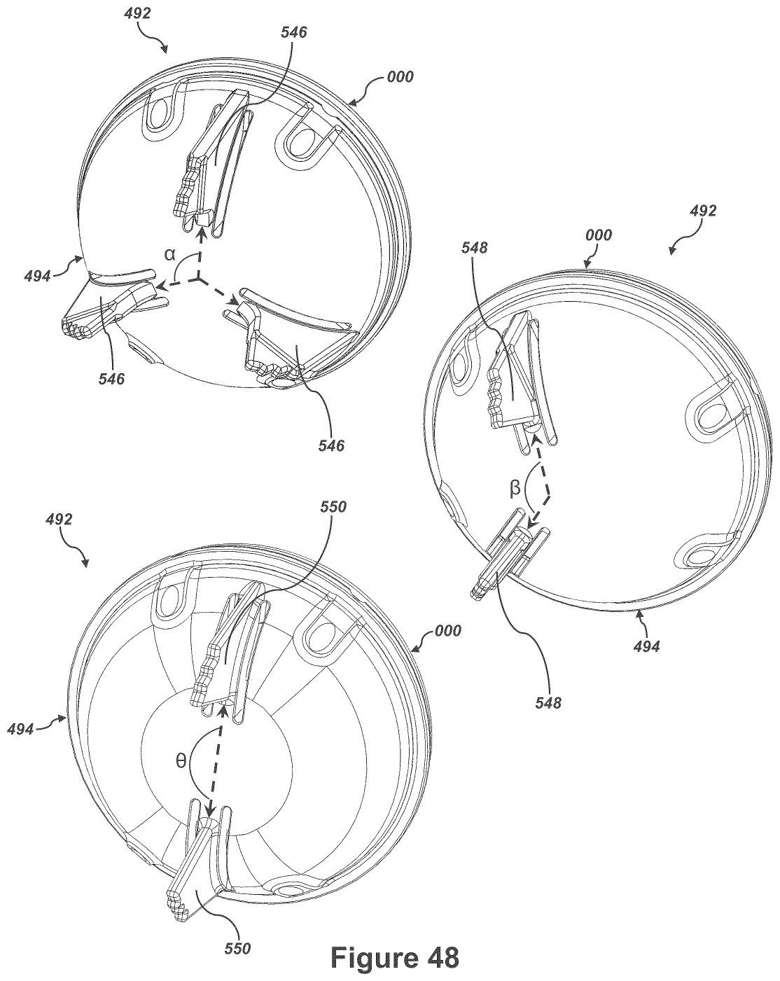

In the views of the embodiment of the disclosure 492 illustrated in FIG. 48, the stability fins 546 can be arranged at an equal angle α (shown as three fins but can be a quantity more than three or less than three) around the circumference, or as two fins 548 arranged at an angle β less than 1800 or two fins 550 arranged at an angle θ equal to 180°.

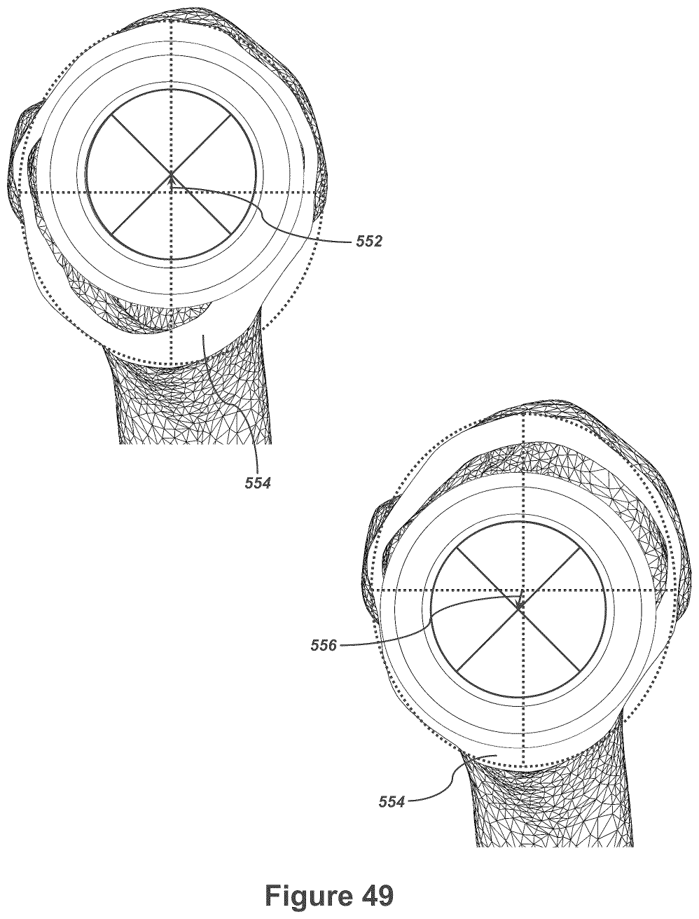

In the views of the embodiment of the disclosure 492 illustrated in FIG. 49, the disclosure can be positioned superiorly a distance 552 away from the central axis of the anatomical cut 554 or positioned inferiorly a distance 556 away from the central axis of the anatomical cut 554.

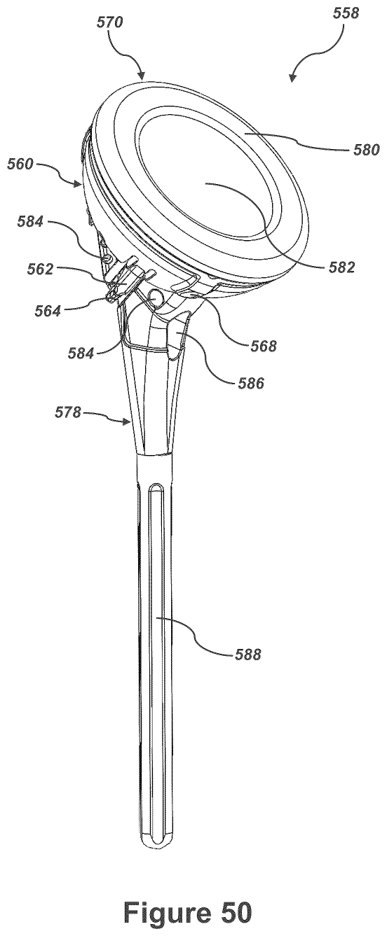

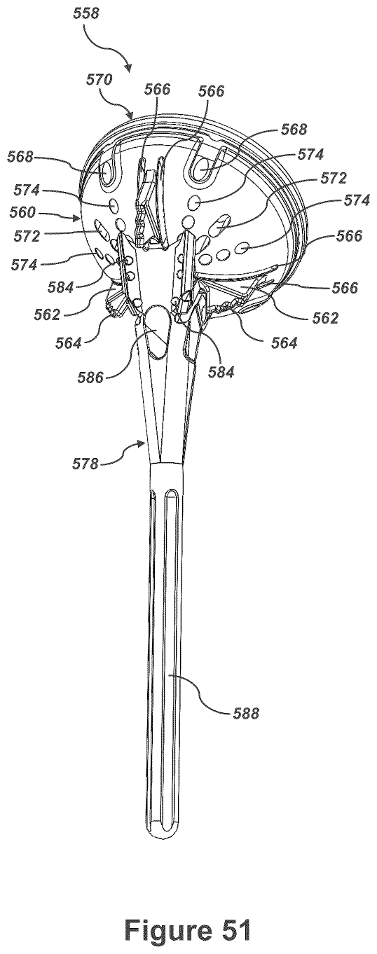

A fourteenth embodiment of the disclosure 558 is illustrated in FIGS. 50-53. As shown therein, a stemless shell 560, stability fins 562 with serrated teeth 564 on their ends, slots/openings 566 which can accept various devices for cutting bone, such as a saw blade, burr, or other suitable cutting instrument, features 568 for passing various types of sutures or attachment sites for a device to apply compression to liner 570, features 572 to apply suction, features 574 to administer bone cement, allograft, bone substitute, or another suitable biocompatible material, and a cavity 576 can accept humeral stem 578, or another biocompatible device. The liner 570 includes a shoulder portion 580, and a concave portion 582. Preferably, the stemless shell 560 is comprised of titanium or another biocompatible metal, and the liner 570 is comprised of a ceramic, ultrahigh molecular weight polyethylene (UHMWPE), or another suitable biocompatible material. Humeral stem 578 includes features 584 for passing various types of sutures or cerclages, a cavity 586 to accept autograft, allograft or another suitable biocompatible material, and a fluted distal region 588 to aid in rotational stability.

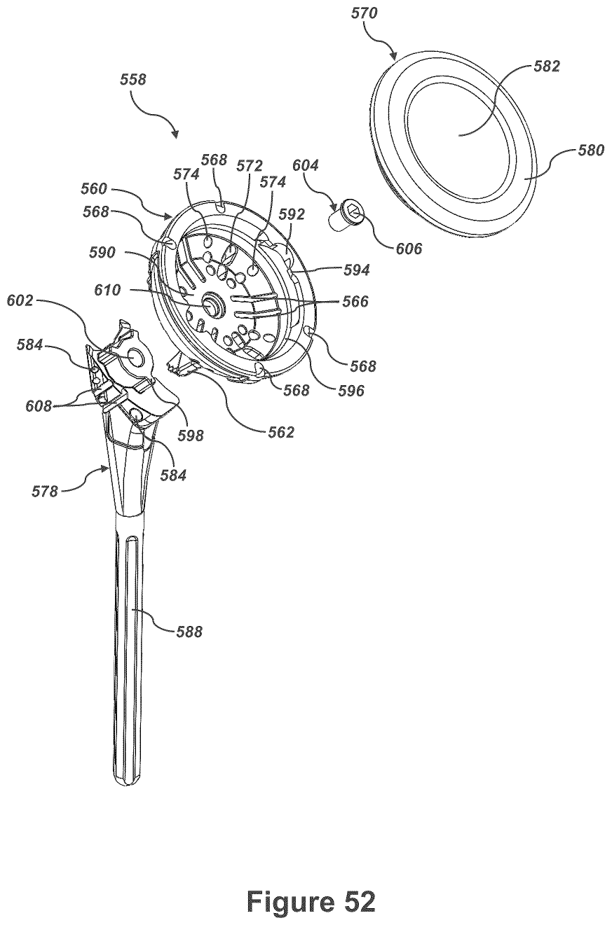

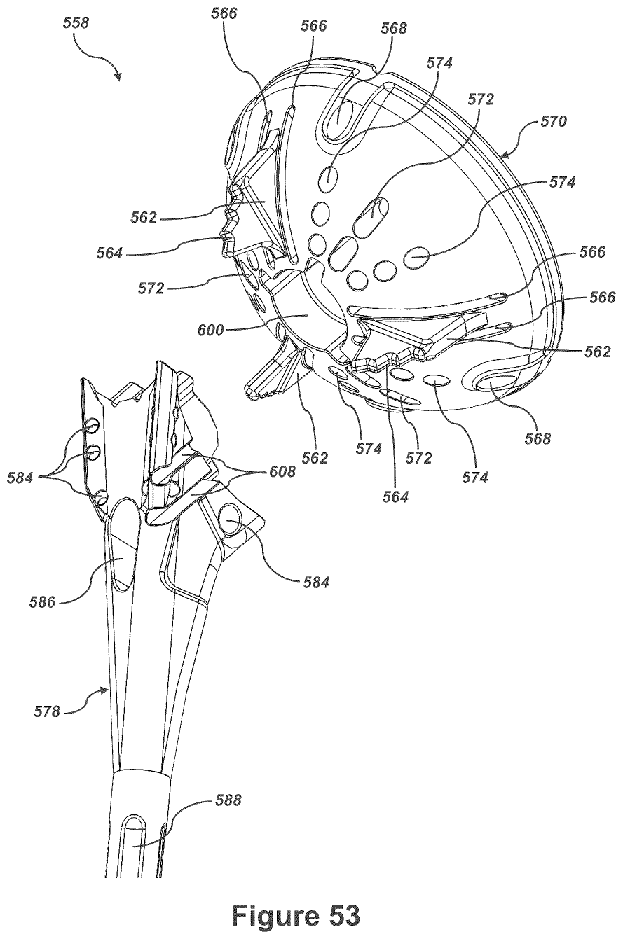

In the exploded view of the embodiment of the disclosure 558 illustrated in FIGS. 52-53, the front cavity portion 590 has features 592, 594 and 596 which allow for attachment of liner 570. Humeral stem 578 has geometry 598 that inserts into cavity 600, a threaded hole 602 that can accept a fastener 604 that includes geometry 606 to interface with a screwdriver, and slots 608 which can accept various devices for cutting bone, such as a saw blade, burr, or other suitable cutting instrument. A stemless shell 560, stability fins 562 with serrated teeth 564 on their ends, slots/openings 566 which can accept various devices for cutting bone, such as a saw blade, burr, or other suitable cutting instrument, features 568 for passing various types of sutures or attachment sites for a device to apply compression to liner 570, features 572 to apply suction, features 574 to administer bone cement, allograft, bone substitute, or another suitable biocompatible material, and a cavity 576 can accept humeral stem 578, or another biocompatible device, and a hole 610 that allows passage for fastener 604. The liner 570 includes a shoulder portion 580, and a concave portion 582.

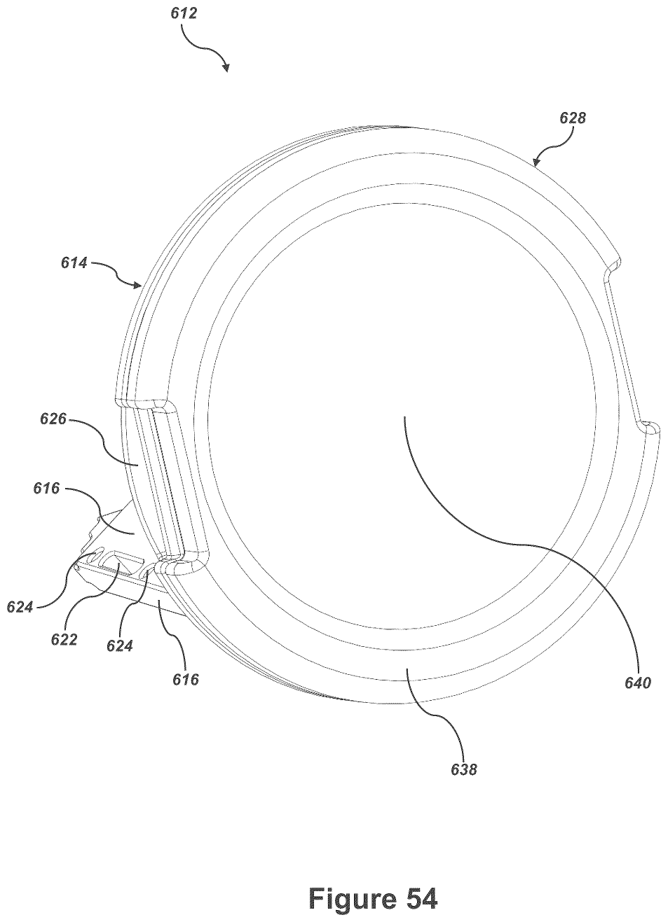

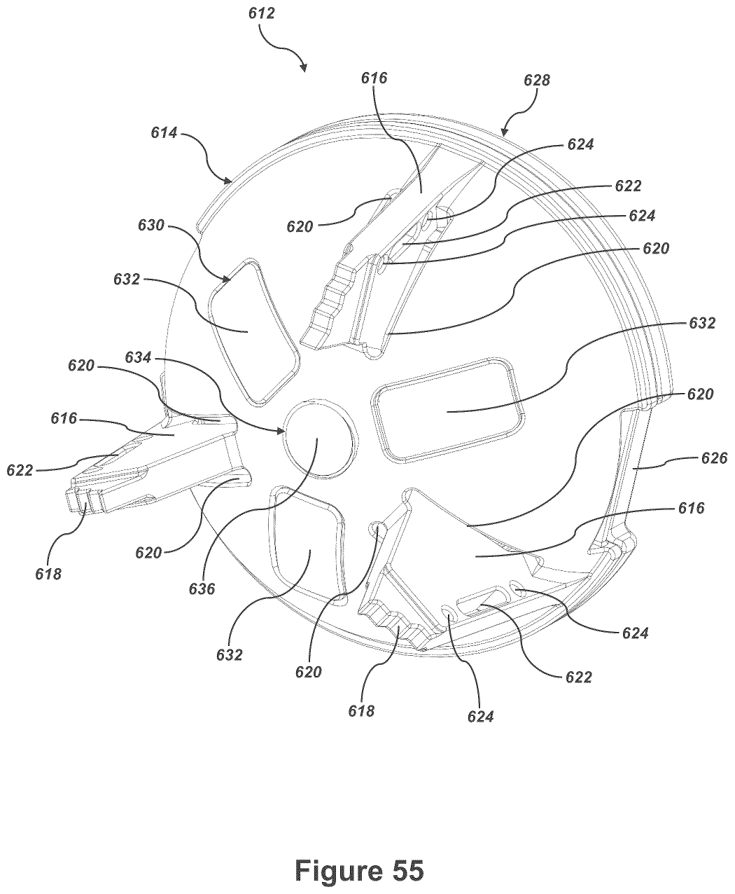

A fifteenth embodiment of the disclosure 612 is illustrated in FIGS. 54-57. As shown therein, a stemless shell 614, stability fins 616 with serrated teeth 618 on their ends, slots/openings 620 which can accept various devices for cutting bone, such as a saw blade, burr, or other suitable cutting instrument, slots 622 and holes 624 for passing various types of sutures, tapes, cerclages or other comparable biocompatible securing devices, attachment sites 626 for a device to apply compression to liner 628, an insert 630 that fits in cavity 632, and which is attached with fastener 634 that threads into hole 636, and cavity 632 can also accept a humeral stem, or another biocompatible device. The liner 628 includes a shoulder portion 638, and a concave portion 640. Preferably, the stemless shell 614 is comprised of titanium or another biocompatible metal, and the liner 628 is comprised of a ceramic, ultrahigh molecular weight polyethylene (UHMWPE), or another suitable biocompatible material.

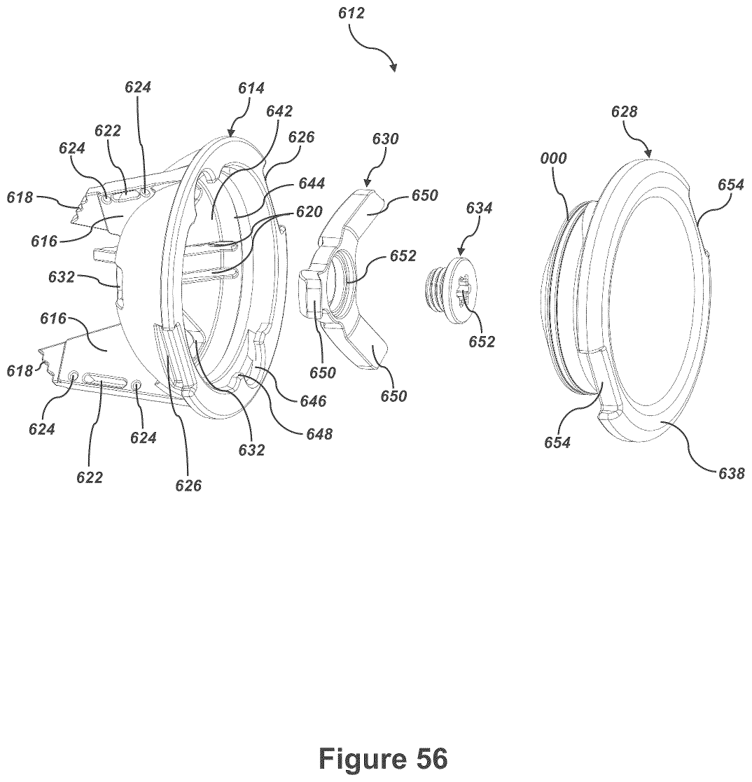

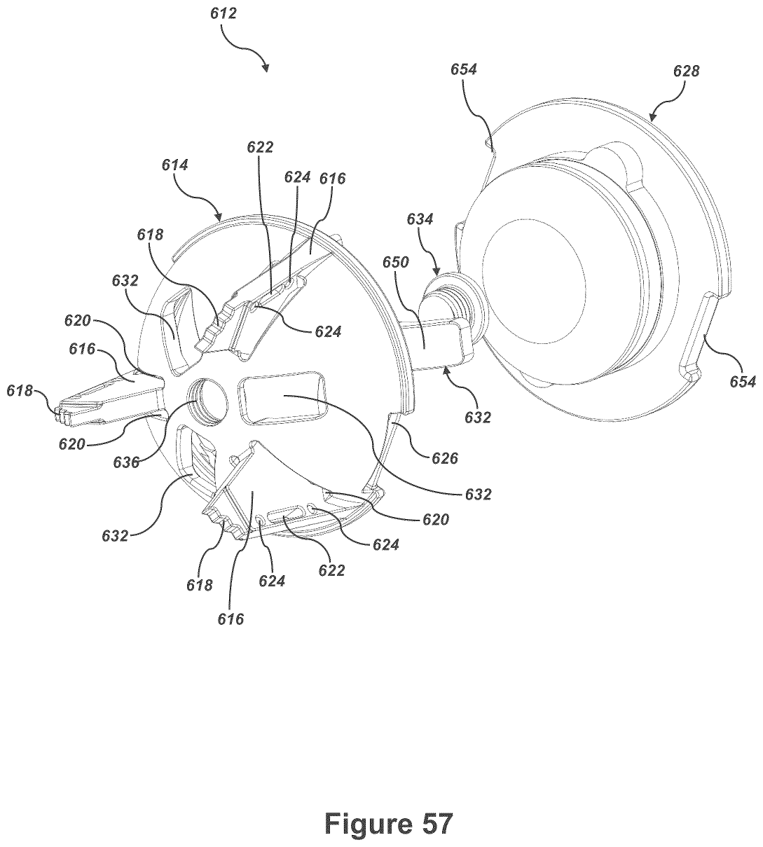

In the exploded view of the embodiment of the disclosure 612 illustrated in FIGS. 56-57, the stemless shell 614 has a front cavity portion 642 and has features 644, 646 and 648 which allow for attachment of liner 628. Insert 630 has geometry 650 that inserts into cavity 632 and has a hole 652 that accepts fastener 634 that attaches to a threaded hole 636 and fastener 634 includes geometry 652 to interface with a screwdriver, and slots 620 which can accept various devices for cutting bone, such as a saw blade, burr, or other suitable cutting instrument. The liner 628 includes a shoulder portion 638, a concave portion 640, and slots 654 to allow passage to a device to apply compression to liner 628.

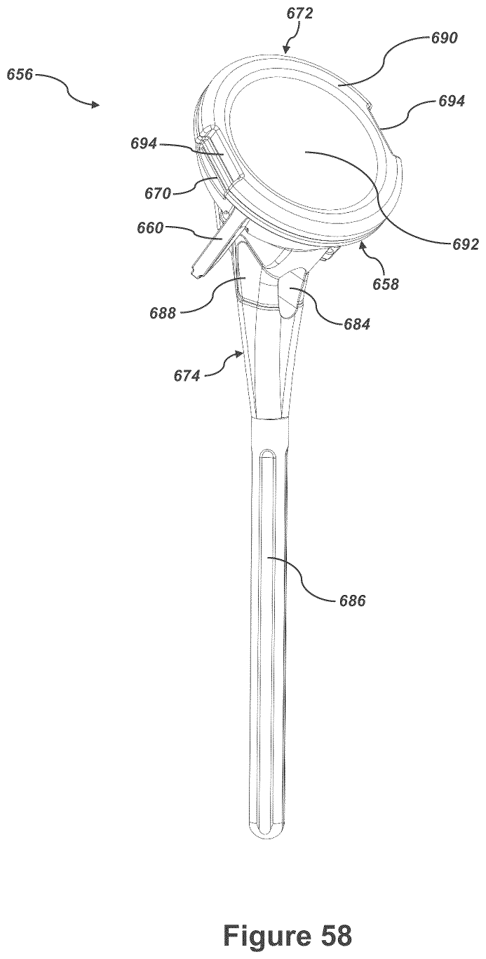

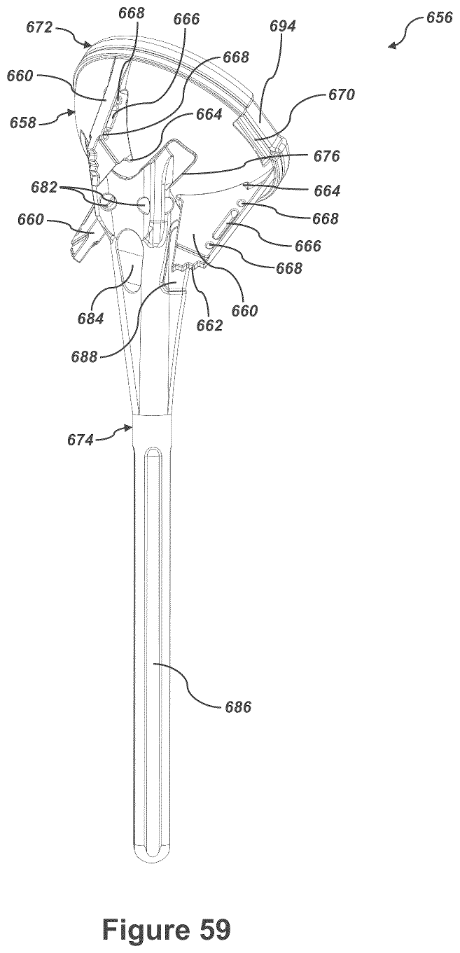

A sixteenth embodiment of the disclosure 656 is illustrated in FIGS. 58-61. As shown therein, a stemless shell 658, stability fins 660 with serrated teeth 662 on their ends, slots/openings 664 which can accept various devices for cutting bone, such as a saw blade, burr, or other suitable cutting instrument, slots 666 and holes 668 for passing various types of sutures, tapes, cerclages or other comparable biocompatible securing devices, attachment sites 670 for a device to apply compression to liner 672, a humeral stem 674 that fits in cavity 676 which can also accept an insert that acts as a filler, or can accept another biocompatible device. The humeral stem 674 includes features 682 for passing various types of sutures or cerclages, a cavity 684 to accept autograft, allograft or another suitable biocompatible material, a fluted distal region 686 to aid in rotational stability, and an irregular surface 688 that allows for bone ingrowth. The liner 672 includes a shoulder portion 690, a concave portion 692, and slots 694 to allow passage of a device to apply compression to liner 672. Preferably, the stemless shell 658 is comprised of titanium or another biocompatible metal, and the liner 672 is comprised of a ceramic, ultrahigh molecular weight polyethylene (UHMWPE), or another suitable biocompatible material.

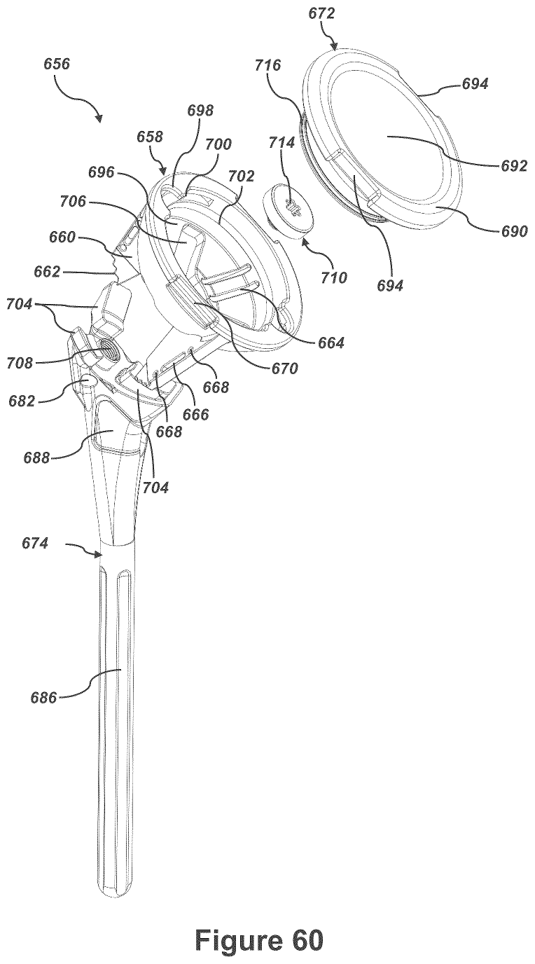

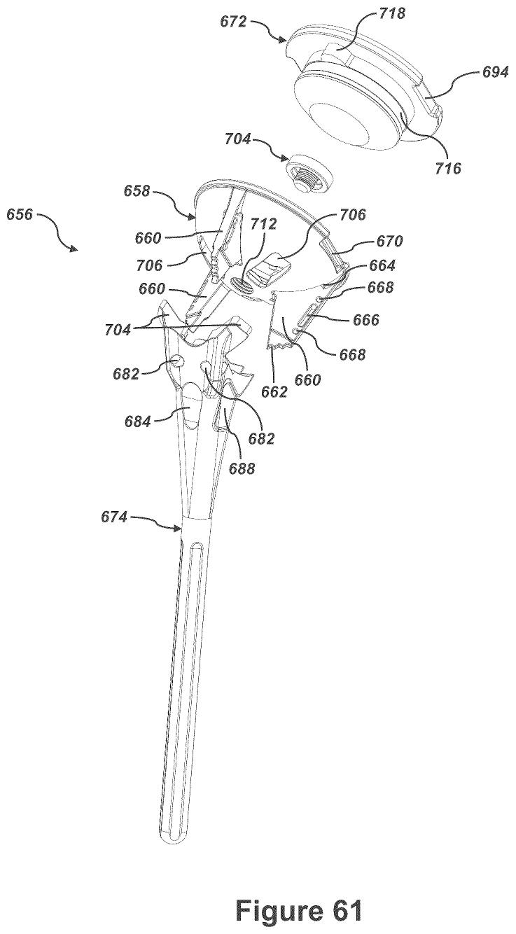

In the exploded view of the embodiment of the disclosure 656 illustrated in FIGS. 60-61, the stemless shell 658 has a front cavity portion 696 and has features 698, 700 and 702 which allow for attachment of liner 672. Humeral stem 674 has geometry 704 that inserts into cavity 706, and it has a threaded hole 708 that accepts fastener 710. Fastener 710 attaches through a threaded hole 712, and includes geometry 714 to interface with a screwdriver. The liner 672 includes a shoulder portion 690, a concave portion 692, features 716 and 718 that aid in attachment of liner 672 to stemless shell 658, and slots 688 to allow passage to a device to apply compression to liner 628.

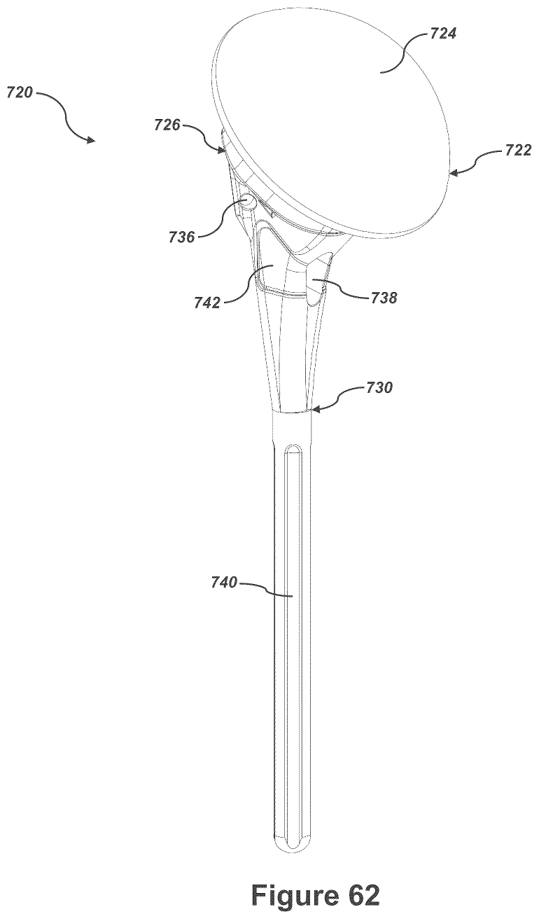

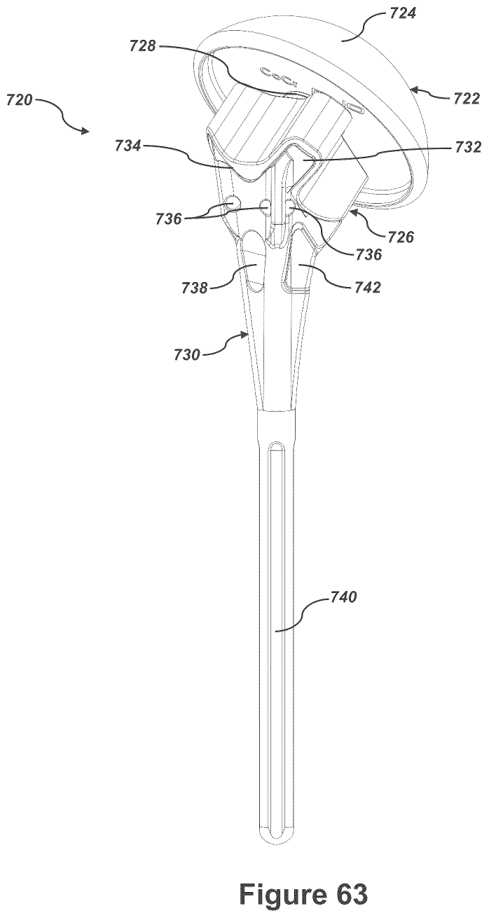

A seventeenth embodiment of the disclosure 720 is illustrated in FIGS. 62-65. As shown therein, a hemispherical head 722 with surface 724 that interacts with a socket and attaches to a spacer 726 using a double tapered component 728, a humeral stem 730 with geometry 732 that fits in cavity 734 which can also accept another biocompatible device. The humeral stem 730 includes features 736 for passing various types of sutures or cerclages, a cavity 738 to accept autograft, allograft or another suitable biocompatible material, a fluted distal region 740 to aid in rotational stability, and an irregular surface 742 that allows for bone ingrowth. Preferably, the hemispherical head 722, the spacer 726, and the humeral stem 730 is comprised of titanium, cobalt chrome, or another biocompatible material.

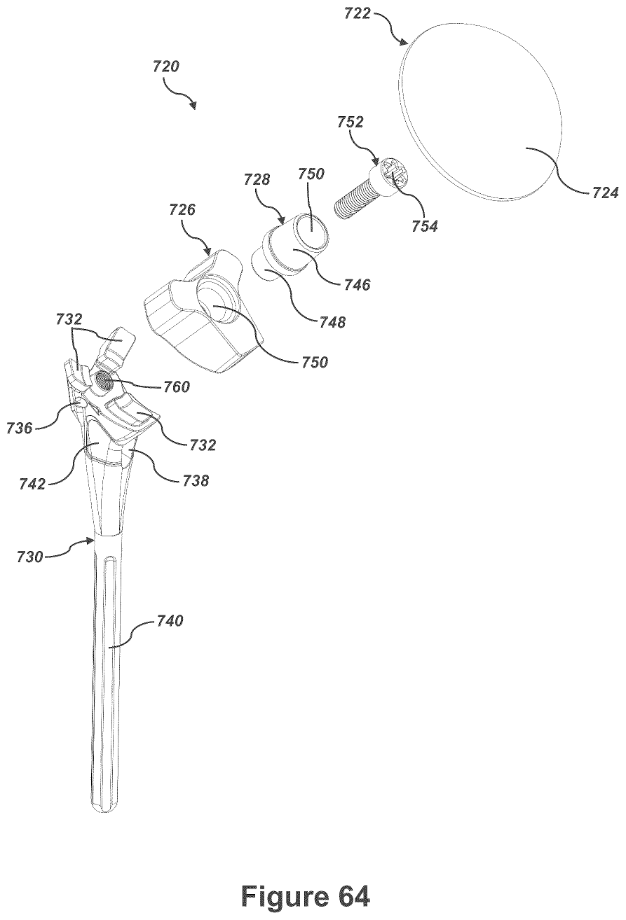

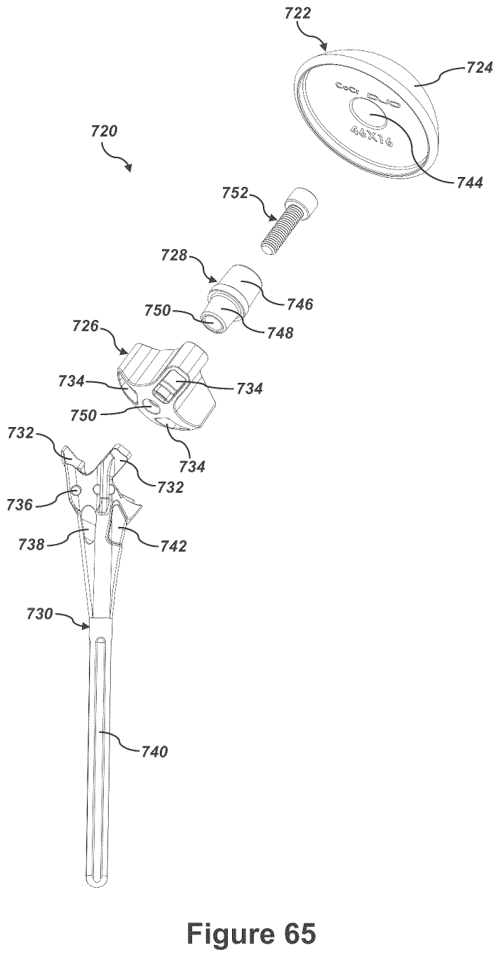

In the exploded view of the embodiment of the disclosure 720 illustrated in FIGS. 64-65, the hemispherical head 722 has a tapered cavity portion 744 which allow for attachment to double tapered component 728. Double tapered component 728 has a large tapered portion 746 and a small tapered portion 748 although both portions can be of the same size, and a cavity 750 that accepts a fastener 752 that has geometry 754 to accept a screwdriver. Spacer 726 has a large cavity 756 that accepts double tapered component 728, and small cavity 758 to attach to the humeral stem 730. Humeral stem 730 has geometry 732 that inserts into cavity 734 on the spacer 726, and it has a threaded hole 760 that accepts fastener 752.

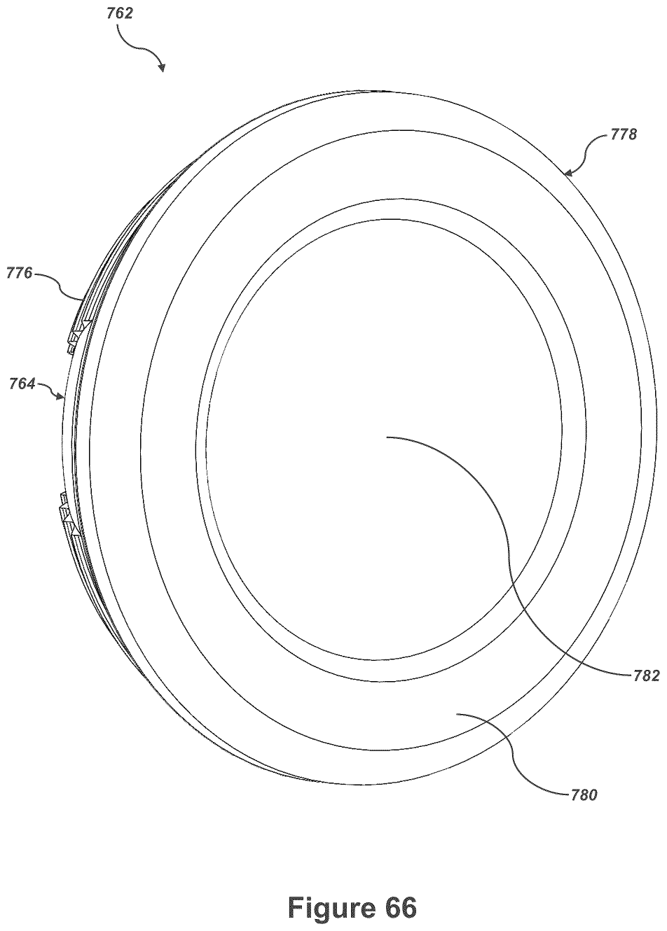

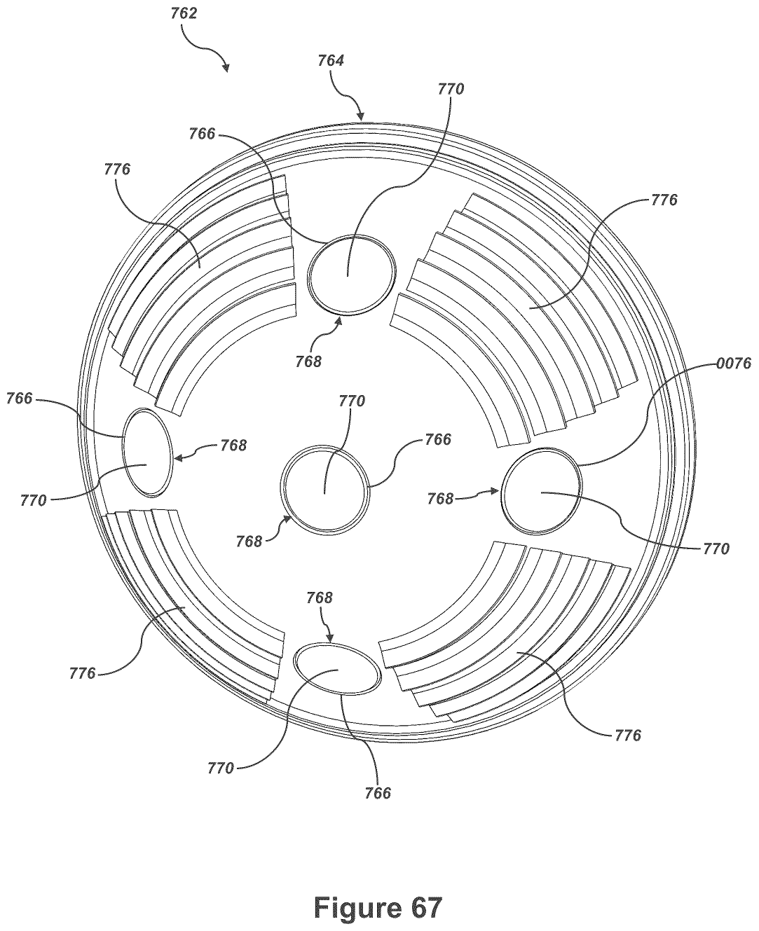

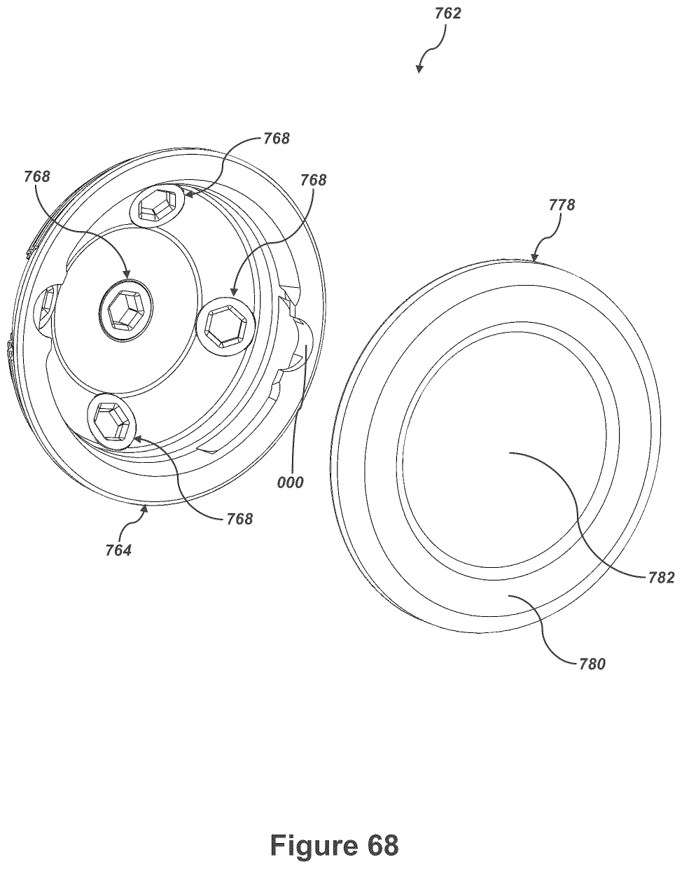

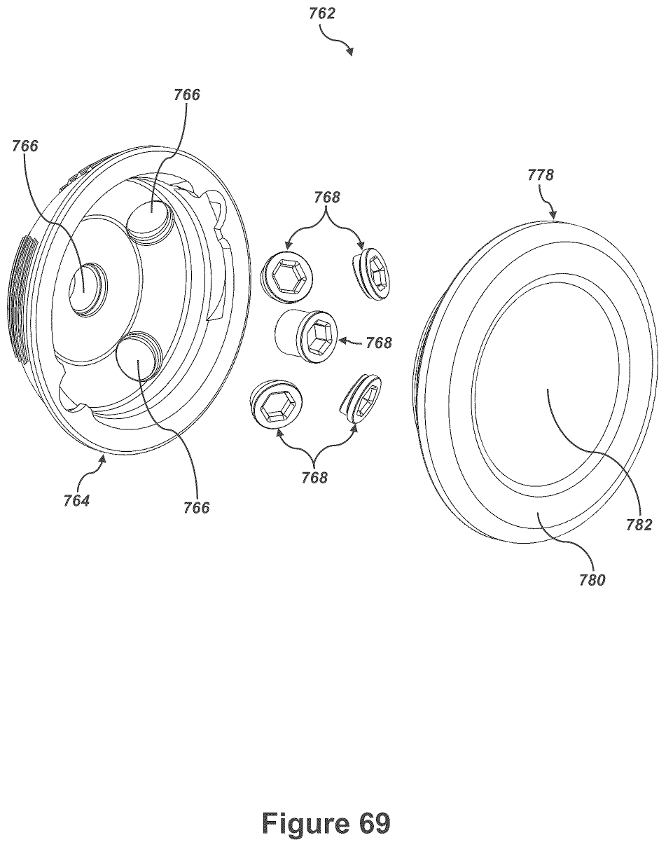

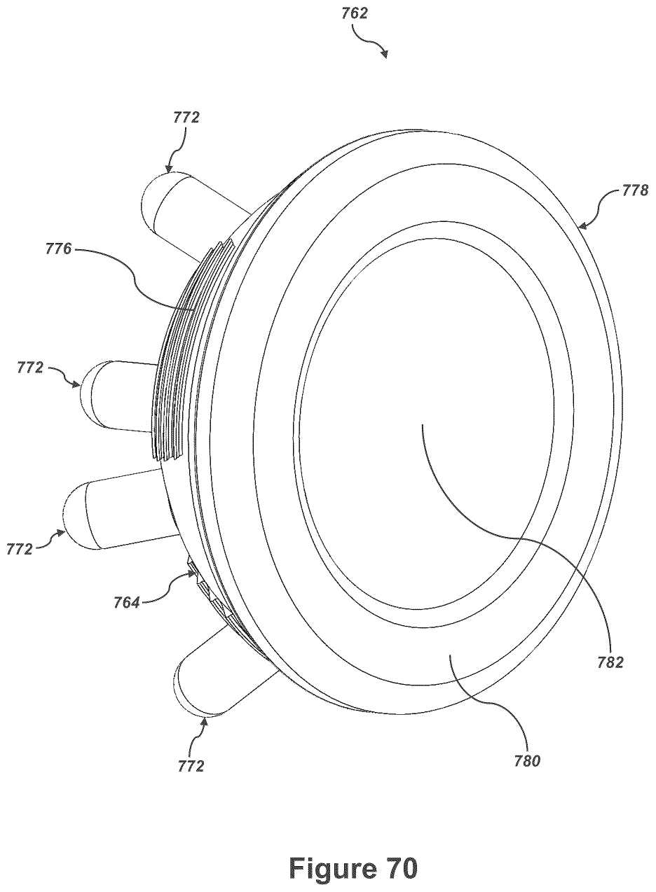

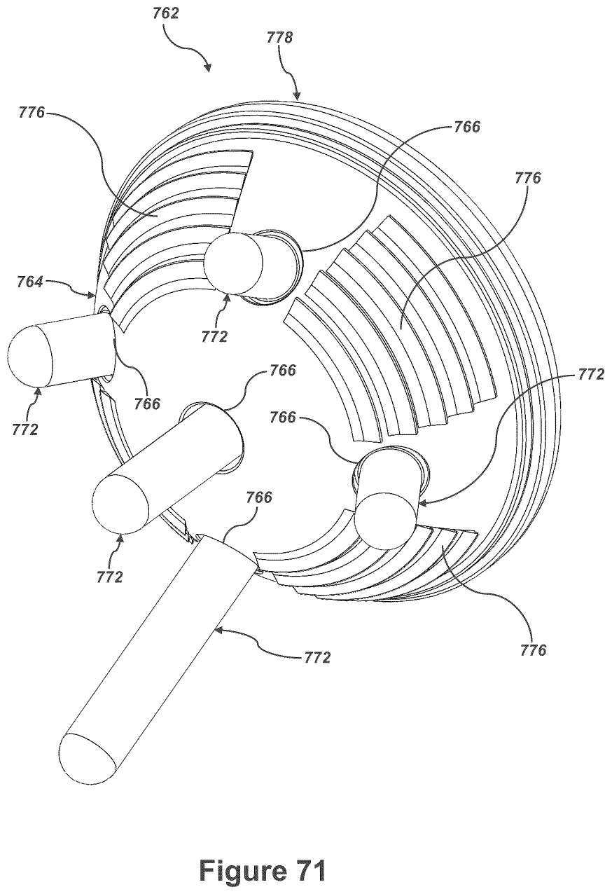

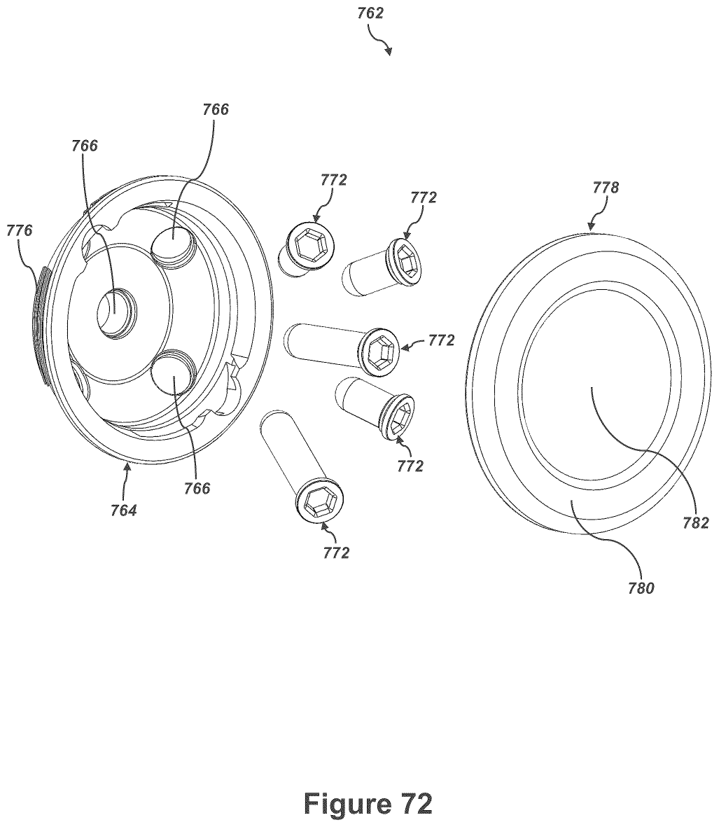

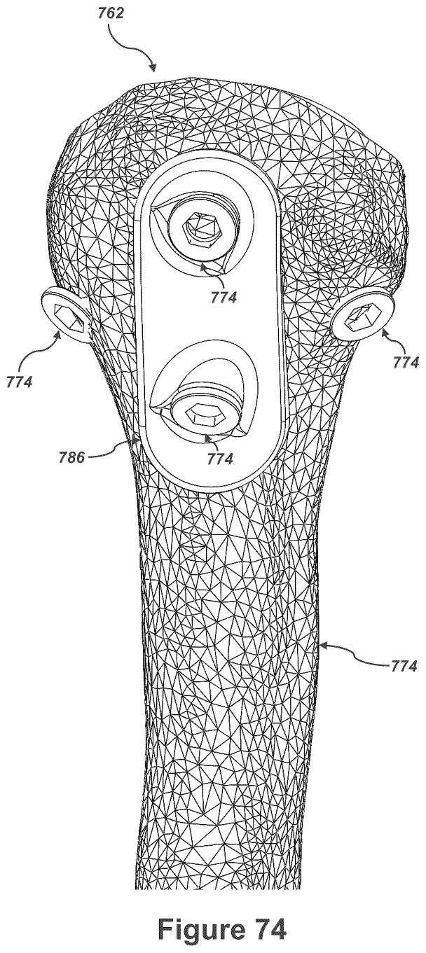

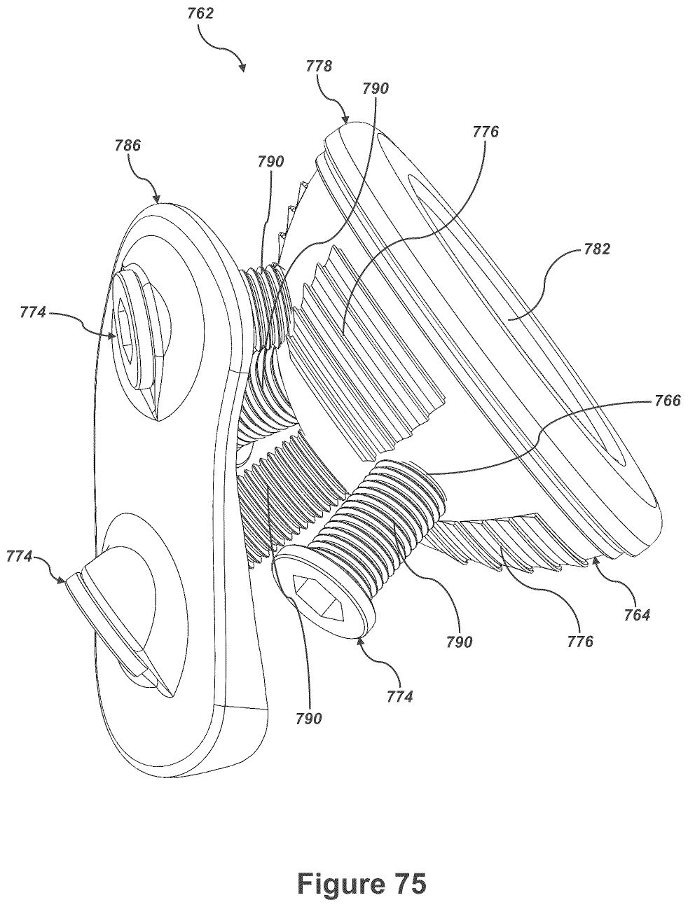

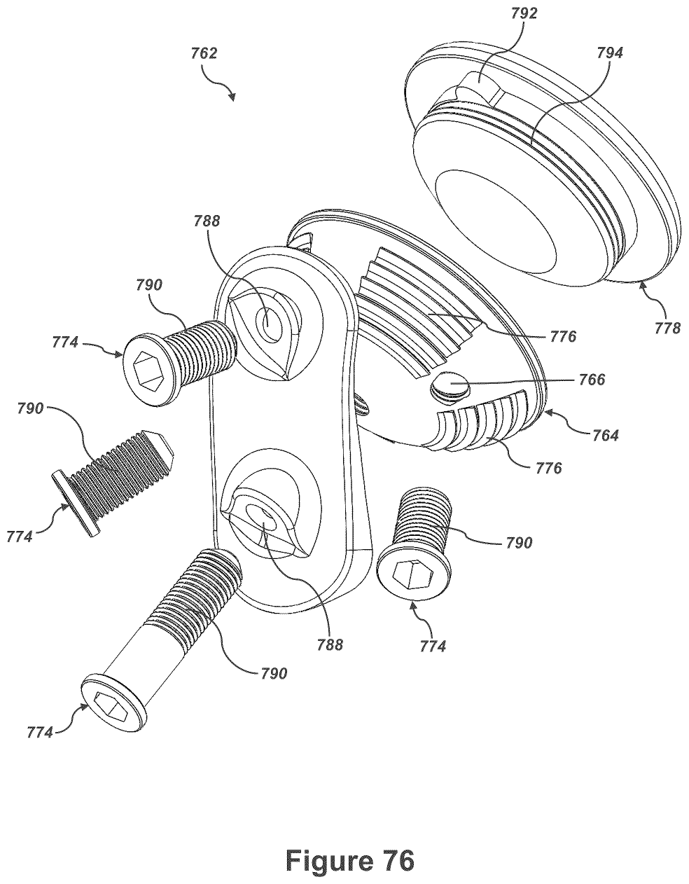

An eighteenth embodiment of the disclosure 762 is illustrated in FIGS. 66-76. As shown therein, a stemless shell 764, with openings 766 which can accept various components including a filler cap 768 with a surface 770 that match the curvature of the stemless shell 764, pegs 772 or fasteners 774, and grooved features 776 for securing to bone for cutting bone. The liner 778 includes a shoulder portion 780, and a concave portion 782. Preferably, the stemless shell 764 is comprised of titanium or another biocompatible metal, and the liner 778 is comprised of a ceramic, ultrahigh molecular weight polyethylene (UHMWPE), or another suitable biocompatible material.

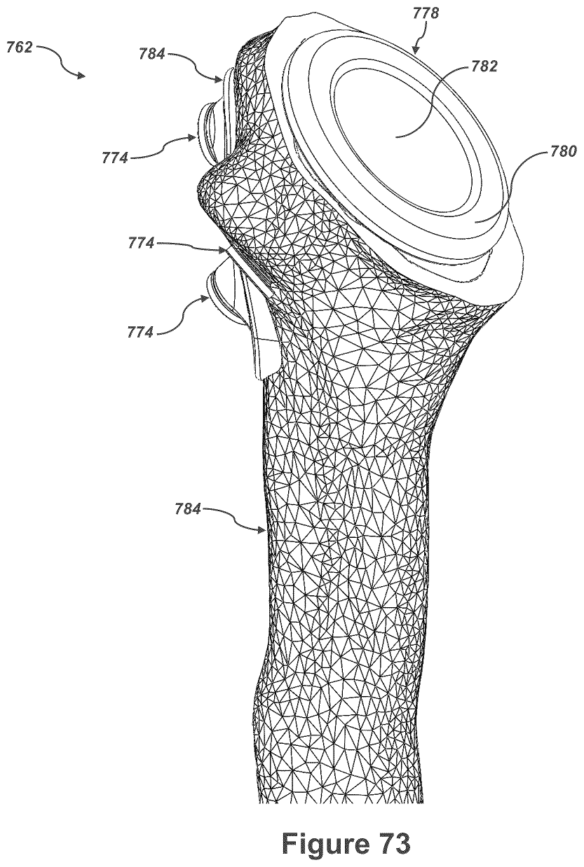

In the views of the embodiment of the disclosure 720 illustrated in FIGS. 73-76, the stemless shell 764 is shown implanted in a humerus 784 and can be buttressed by a plate 786 with openings 788 that allow for attachment with fasteners 774 with threads 790. The liner 778 includes features 792 and 794 to attach to stemless shell 764.

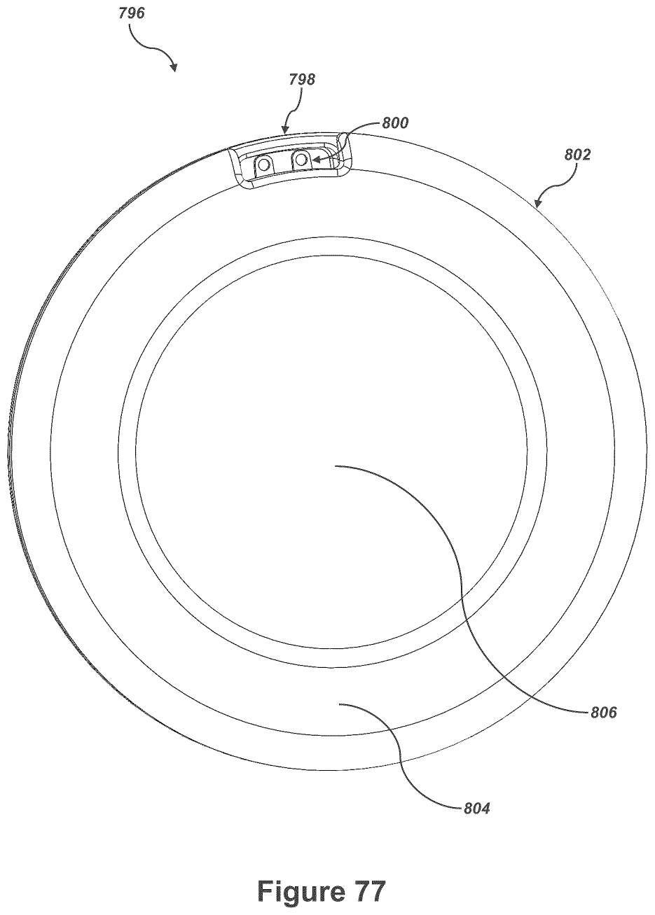

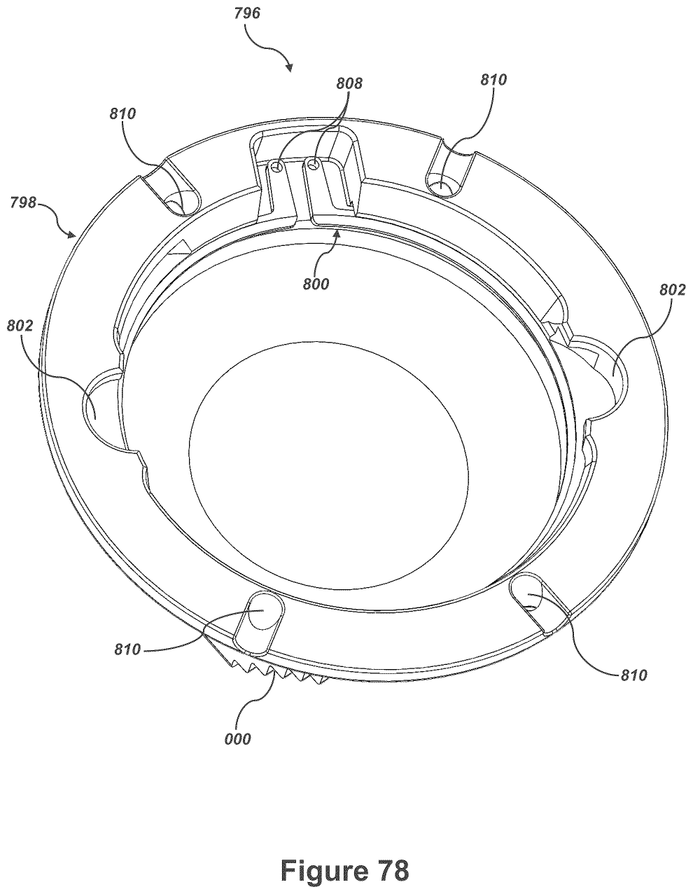

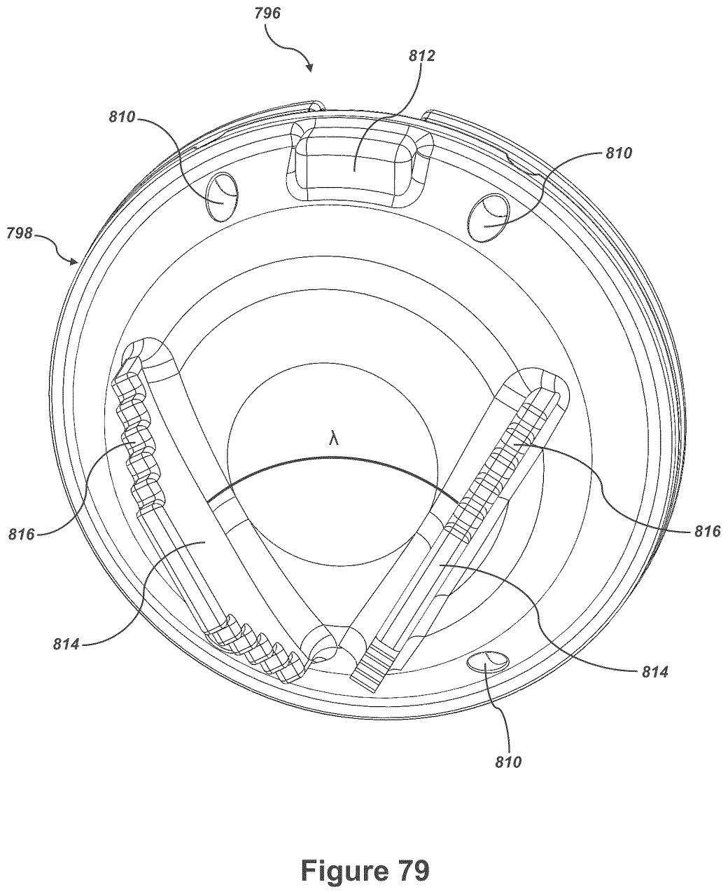

A nineteenth embodiment of the disclosure 796 is illustrated in FIGS. 77-80. As shown therein, a stemless shell 798 with a snap ring 800 to attach a liner 802 that includes a shoulder portion 804, and a concave portion 806 to interface with a ball. The snap ring 800 has openings 808 to attach a device to actuate snap ring so as to detach liner 802 from stemless shell 798. The stemless shell 798 includes openings 810 for passing various types of sutures or attachment sites for a device to apply compression to liner 802, and features to secure liner 802. The underside of the stemless shell 798 includes a feature 812 to allow space for snap ring 800 to be actuated, and stability fins 814 angled at λ to each other with serrated teeth 816 on their ends. Preferably, the stemless shell 798 is comprised of titanium or another biocompatible metal, and the liner 802 is comprised of a ceramic, ultrahigh molecular weight polyethylene (UHMWPE), or another suitable biocompatible material.

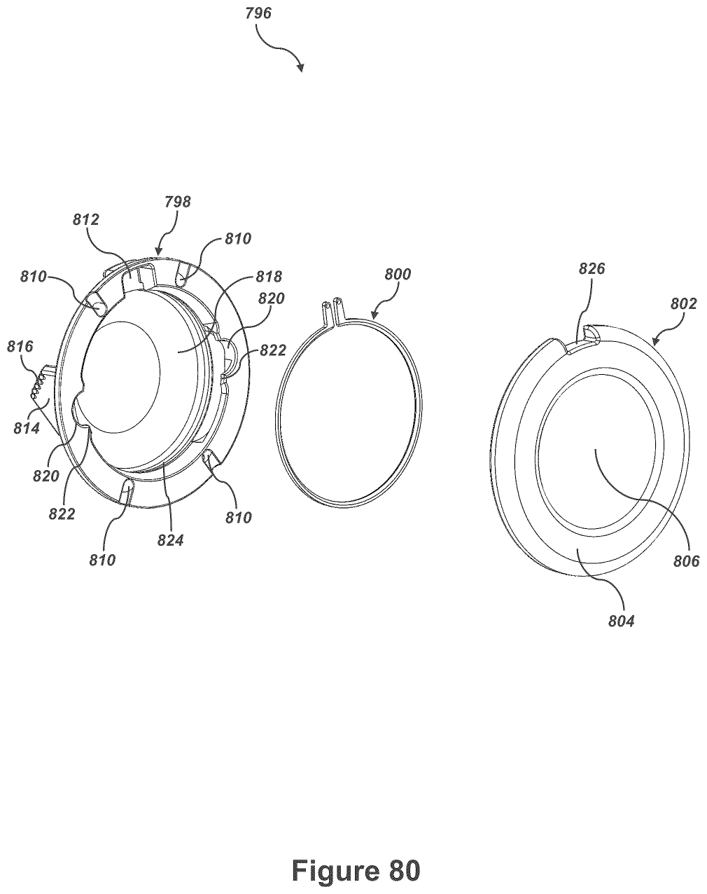

In the exploded view of the embodiment of the disclosure 796 illustrated in FIG. 80, the stemless shell 798 has a front cavity portion 818 with features 820, 822 and 824 which allow for attachment of liner 802. The liner 802 attaches to stemless shell 798 using a snap ring 800 and has an opening 826 to allow access to actuating snap ring 800.

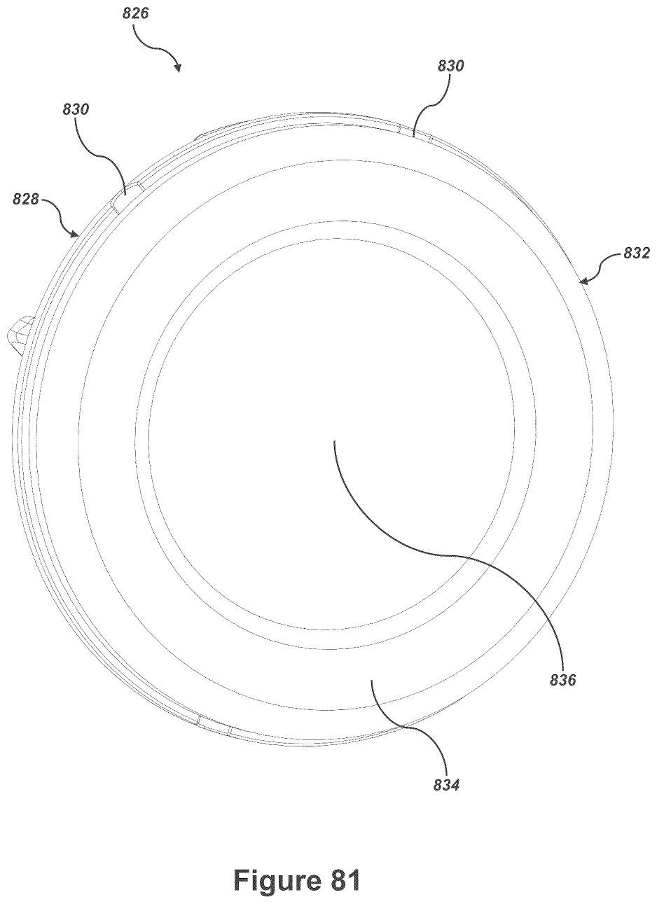

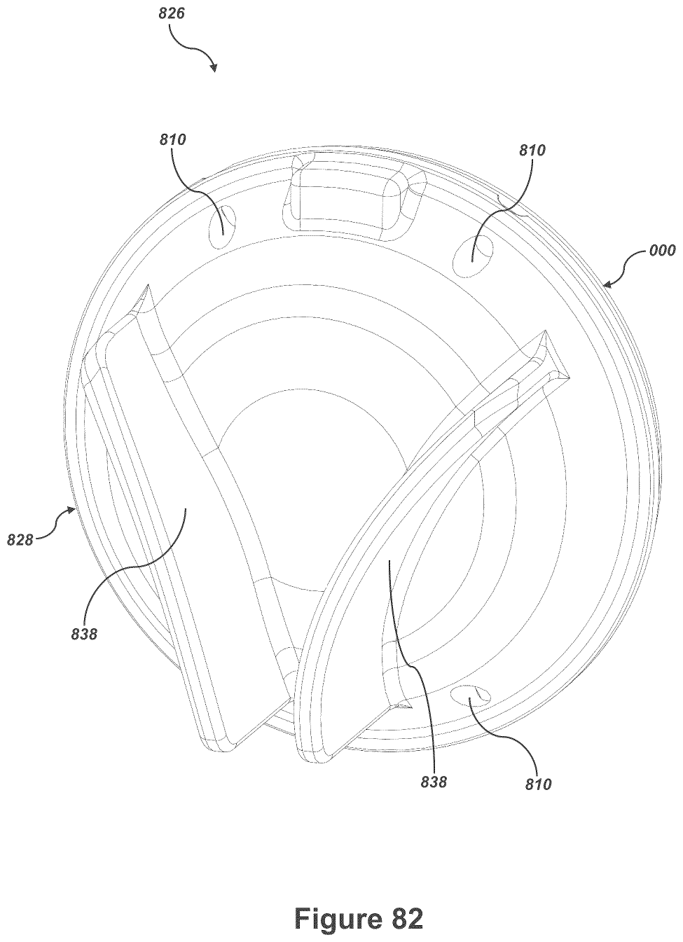

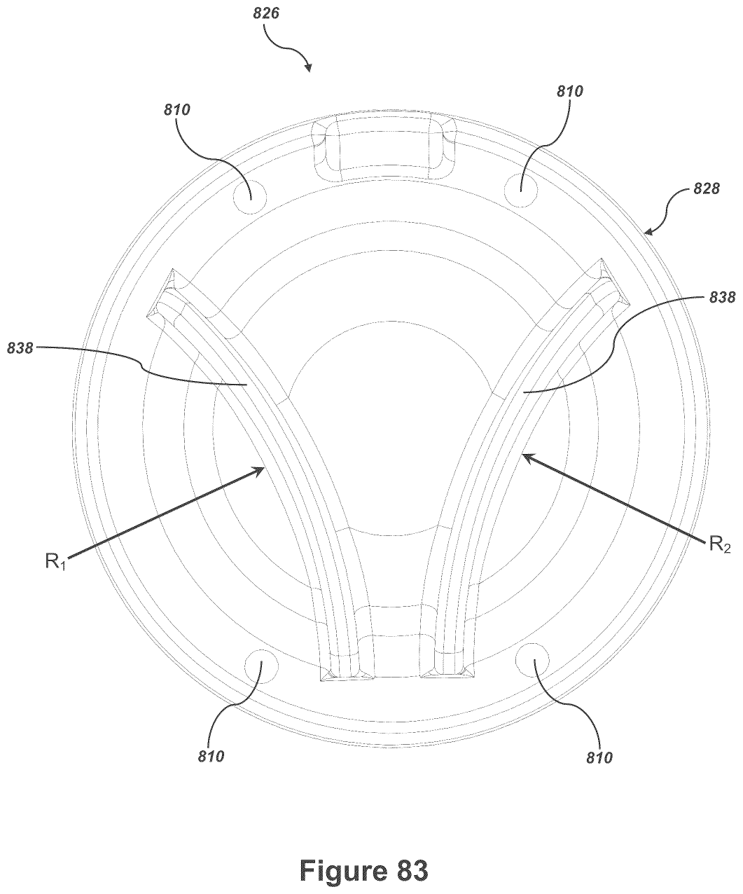

A twentieth embodiment of the disclosure 826 is illustrated in FIGS. 81-83. As shown therein, a stemless shell 828 includes openings 830 for passing various types of sutures or attachment sites for a device to apply compression to liner 832, and which includes a shoulder portion 834, and a concave portion 836 to interface with a ball. The underside of the stemless shell 828 includes stability fins 838 curved at a radius of R₁ or R₂, where R₁ and R₂ can be equal to or different from each other. Preferably, the stemless shell 828 is comprised of titanium or another biocompatible metal, and the liner 832 is comprised of a ceramic, ultrahigh molecular weight polyethylene (UHMWPE), or another suitable biocompatible material.

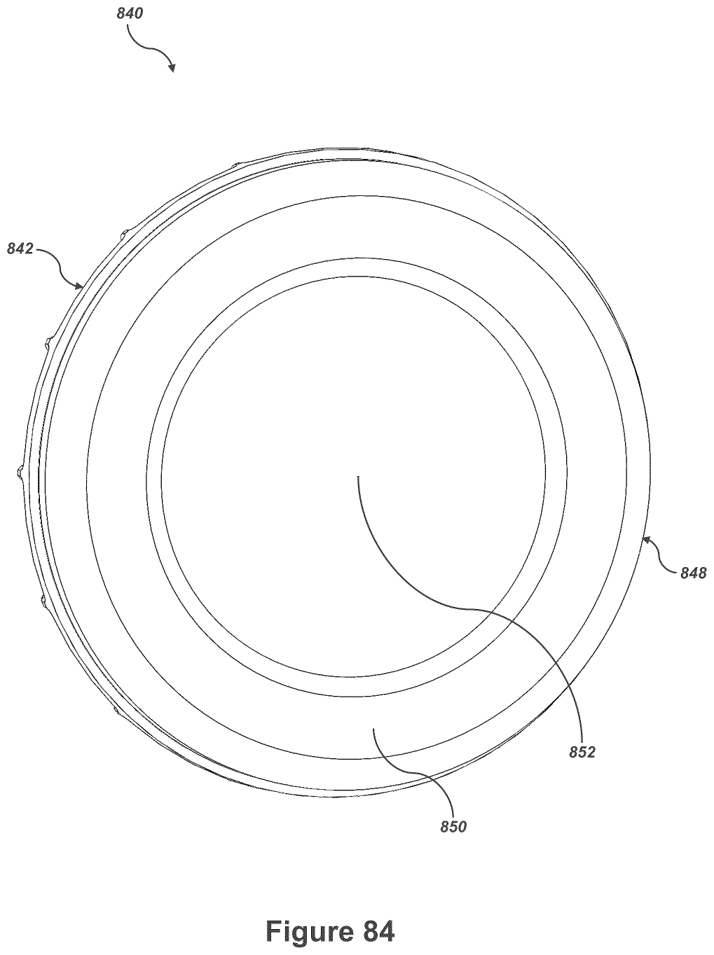

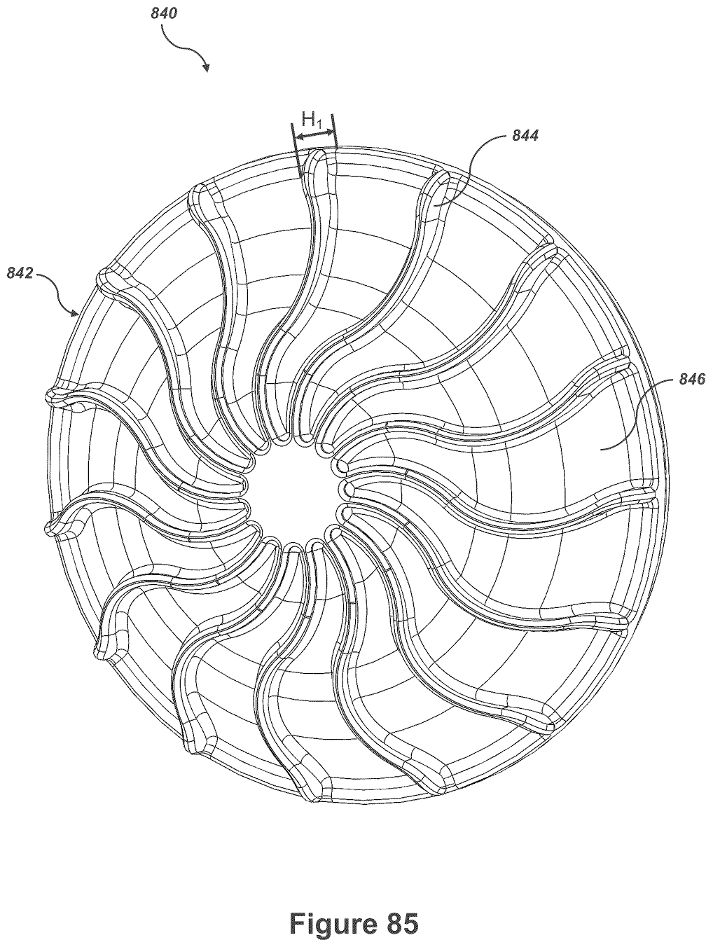

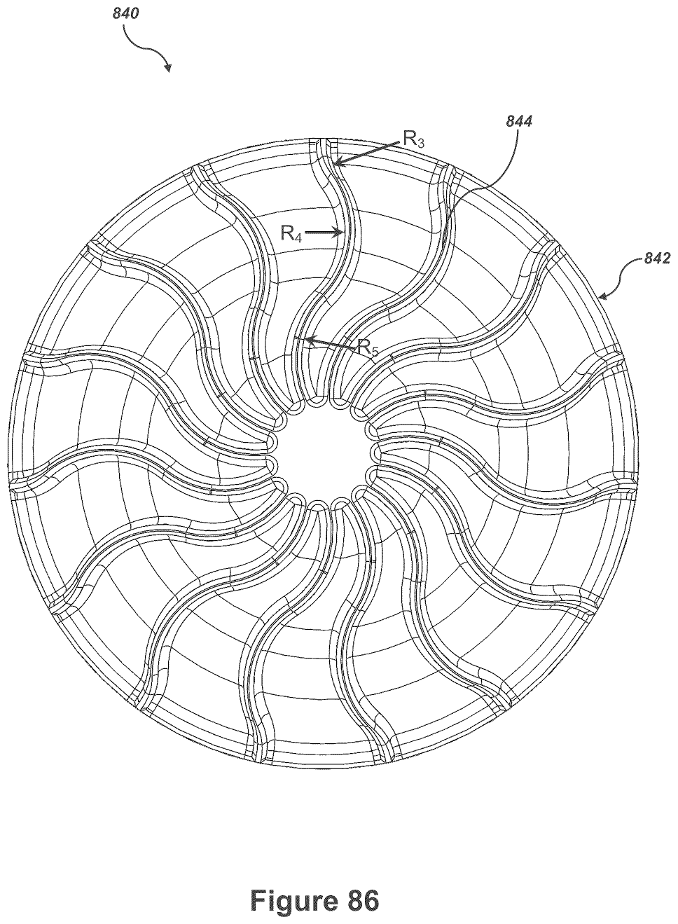

A twenty-first embodiment of the disclosure 840 is illustrated in FIGS. 84-87. As shown therein, a stemless shell 842 has stability fins 844 on its underside 846 of constant height H₁ and multiple radii R₃, R₄ and R₅, where R₃, R₄ and R₅ can be equal to or different from each other and can be less than or more than 3 different radii. The stability fins 844 are shown in a quantity of 15 in FIGS. 85 and 86, but they can be present in more or less than a quantity of 15. The liner 848 includes a shoulder portion 850, and a concave portion 852 to interface with a ball. Preferably, the stemless shell 842 is comprised of titanium or another biocompatible metal, and the liner 848 is comprised of a ceramic, ultrahigh molecular weight polyethylene (UHMWPE), or another suitable biocompatible material.

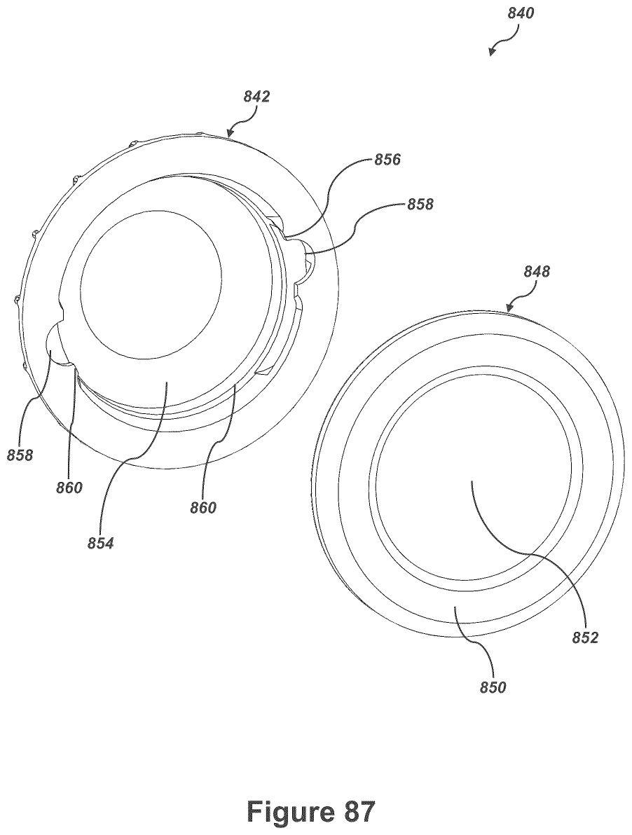

In the exploded view of the embodiment of the disclosure 840 illustrated in FIG. 87, the stemless shell 842 has a front cavity portion 854 with features 856, 858 and 860 which allow for attachment of liner 848.

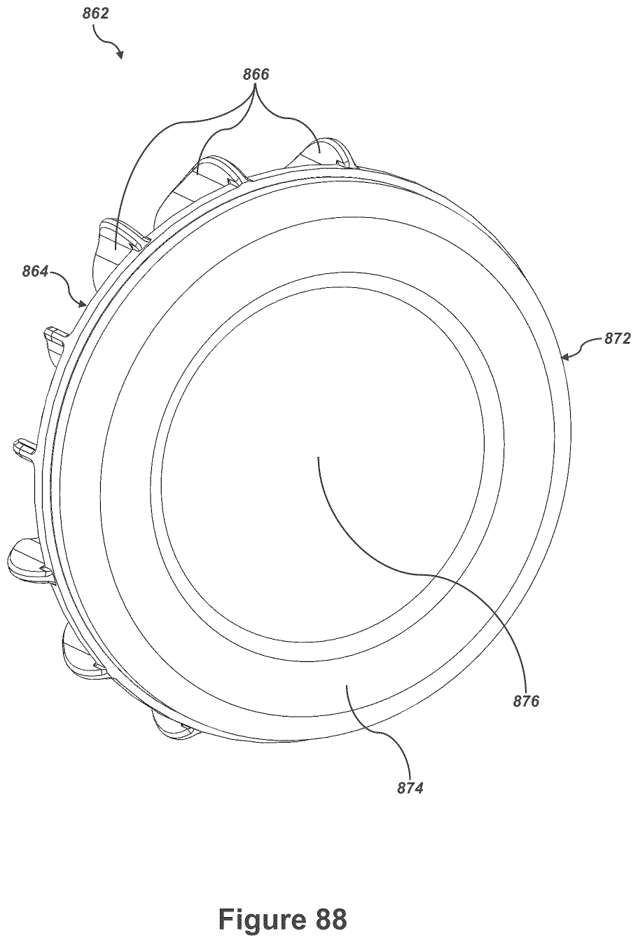

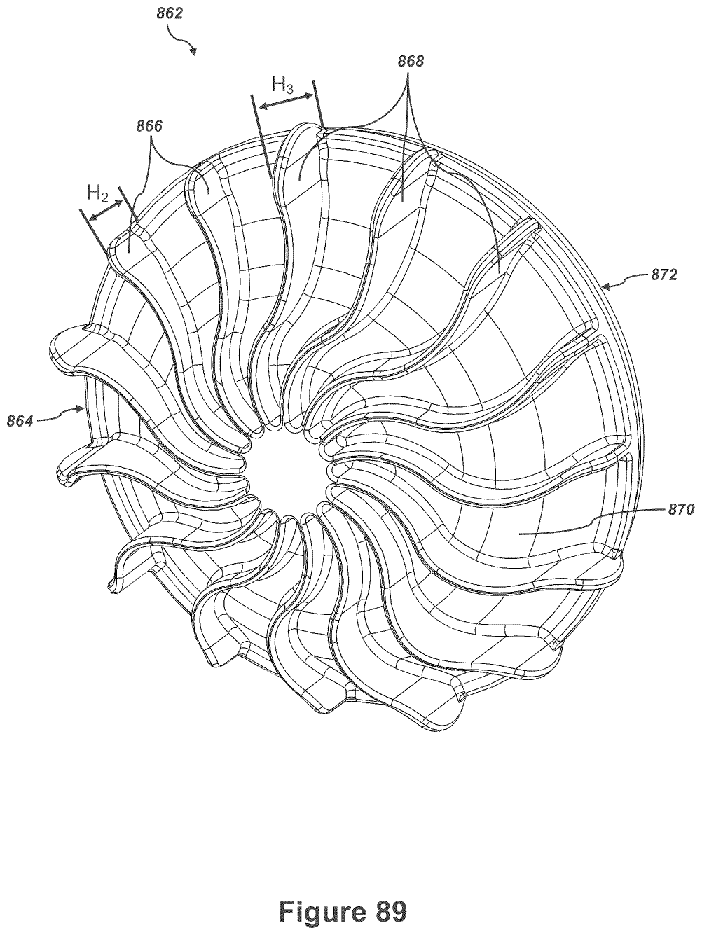

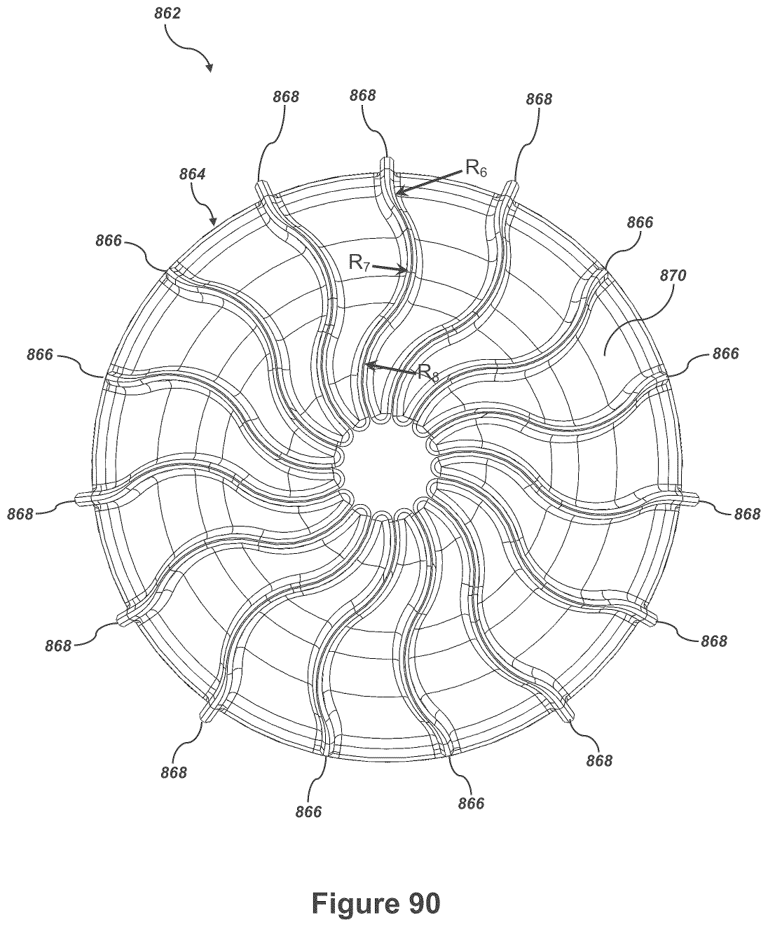

A twenty-second embodiment of the disclosure 862 is illustrated in FIGS. 88-91. As shown therein, a stemless shell 864 has stability fins 866 and 868 on its underside 870 of variable height H₂ and H₃ and multiple radii R₆, R₇ and R₈, where R₆, R₇ and R₈ can be equal to or different from each other and can be less than or more than 3 different radii. The stability fins 864 are shown in a quantity of 15 in FIGS. 89 and 90, but they can be present in more or less than a quantity of 15. The liner 872 includes a shoulder portion 874, and a concave portion 876 to interface with a ball. Preferably, the stemless shell 864 is comprised of titanium or another biocompatible metal, and the liner 872 is comprised of a ceramic, ultrahigh molecular weight polyethylene (UHMWPE), or another suitable biocompatible material.

In the exploded view of the embodiment of the disclosure 862 illustrated in FIG. 91, the stemless shell 864 has a front cavity portion 878 with features 880, 882 and 884 which allow for attachment of liner 872.

The principles, preferred embodiments and modes of operation of the present disclosure have been made apparent in the foregoing description.

Although the embodiments are numbered with, for example, “first,” “second,” “third,” or “fourth,” the ordinal numbers do not imply priorities of the embodiments.

Since many modifications, variations and changes in detail can be made to the described embodiments of the disclosure, it is intended that all matters in the foregoing description and shown in the accompanying drawings be interpreted as illustrative and not in a limiting sense. Thus, the scope of the disclosure should be determined by the appended claims and their legal equivalents.

### ALTERNATIVE IMPLEMENTATIONS

Implementation 1. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion.

Implementation 2. The humeral component according to implementation 1 wherein the distal end of stemless portion includes a plurality of fins, each of which is provided with at least one serrated tooth along its surface.

Implementation 3. The humeral component according to implementation 1 wherein the stemless portion includes at least one through hole.

Implementation 4. The humeral component according to implementation 1 wherein the proximal portion of the distal end of the stemless portion is curved.

Implementation 5. The humeral component according to implementation 1 wherein the distal portion of the distal end of the stemless portion is cylindrical.

Implementation 6. The humeral component according to implementation 1 wherein the distal surface of the stemless portion is osteoinductive.

Implementation 7. The humeral component according to implementation 1 wherein the stemless portion has a cavity for bone grafting.

Implementation 7. The humeral component according to implementation 1 wherein the distal end of the liner portion is irregularly shaped.

Implementation 9. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion.

Implementation 10. The humeral component according to implementation 9 wherein the distal end of stemless portion includes a plurality of fins, each of which is provided with at least one serrated tooth along its surface.

Implementation 11. The humeral component according to implementation 9 wherein the stemless portion includes at least one through hole.

Implementation 12. The humeral component according to implementation 9 wherein the proximal portion of the distal end of the stemless portion is cylindrical.

Implementation 13. The humeral component according to implementation 9 wherein the distal portion of the distal end of the stemless portion is cylindrical.

Implementation 14. The humeral component according to implementation 9 wherein the distal surface of the stemless portion is osteoinductive.

Implementation 15. The humeral component according to implementation 9 wherein the stemless portion has a cavity for bone grafting.

Implementation 16. The humeral component according to implementation 9 wherein the distal end of the liner portion is irregularly shaped.

Implementation 17. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion.

Implementation 18. The humeral component according to implementation 17 wherein the distal end of stemless portion includes a plurality of lobes.

Implementation 19. The humeral component according to implementation 17 wherein the stemless portion includes at least one through hole.

Implementation 20. The humeral component according to implementation 17 wherein the proximal portion of the distal end of the stemless portion is curved.

Implementation 21. The humeral component according to implementation 17 wherein the distal portion of the distal end of the stemless portion terminates at an angle.

Implementation 22. The humeral component according to implementation 17 wherein the distal portion of the distal end of the stemless portion has lobes of varying lengths.

Implementation 23. The humeral component according to implementation 17 wherein the distal surface of the stemless portion is osteoinductive.

Implementation 24. The humeral component according to implementation 17 wherein the stemless portion has a cavity for bone grafting.

Implementation 25. The humeral component according to implementation 17 wherein the distal end of the liner portion is irregularly shaped.

Implementation 26. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion, a fastener portion having a proximal end and a distal end, said distal end adapted for insertion into proximal end of stemless portion.

Implementation 27. The humeral component according to implementation 26 wherein the distal end of stemless portion includes a plurality of lobes.

Implementation 28. The humeral component according to implementation 26 wherein the stemless portion includes at least one through hole.

Implementation 29. The humeral component according to implementation 26 wherein the stemless portion accepts a fastener to compress stemless portion into bone.

Implementation 30. The humeral component according to implementation 26 wherein the proximal portion of the distal end of the stemless portion is curved.

Implementation 31. The humeral component according to implementation 26 wherein the distal portion of the distal end of the stemless portion is curved.

Implementation 32. The humeral component according to implementation 26 wherein the distal portion of the distal end of the stemless portion has lobes of the same length.

Implementation 33. The humeral component according to implementation 26 wherein the distal surface of the stemless portion is osteoinductive.

Implementation 34. The humeral component according to implementation 26 wherein the stemless portion has a cavity for bone grafting.

Implementation 35. The humeral component according to implementation 26 wherein the distal end of the liner portion is irregularly shaped.

Implementation 36. The humeral component according to implementation 26 wherein the fastener has an osteoinductive distal surface.

Implementation 37. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion, a fastener portion having a proximal end and a distal end, said distal end adapted for insertion into proximal end of stemless portion.

Implementation 38. The humeral component according to implementation 37 wherein the distal end of stemless portion includes a plurality of lobes.

Implementation 39. The humeral component according to implementation 37 wherein the distal portion of the distal end of the stemless portion is curved.

Implementation 40. The humeral component according to implementation 37 wherein the distal portion of the distal end of the stemless portion terminates at an angle.

Implementation 41. The humeral component according to implementation 37 wherein the distal portion of the distal end of the stemless portion has lobes of varying lengths.

Implementation 42. The humeral component according to implementation 37 wherein the stemless portion includes at least one through hole.

Implementation 43. The humeral component according to implementation 37 wherein the stemless portion accepts a fastener to compress stemless portion into bone.

Implementation 44. The humeral component according to implementation 37 wherein the proximal portion of the distal end of the stemless portion is curved.

Implementation 46. The humeral component according to implementation 37 wherein the distal surface of the stemless portion is osteoinductive.

Implementation 47. The humeral component according to implementation 37 wherein the distal end of the liner portion is irregularly shaped.

The humeral component according to implementation 37 wherein the fastener has an osteoinductive distal surface.

Implementation 48. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion.

Implementation 49. The humeral component according to implementation 48 wherein the distal end of stemless portion includes a plurality of fins, each of which is provided with at least one serrated tooth along its surface.

Implementation 50. The humeral component according to implementation 49 wherein the plurality of fins are of different lengths.

Implementation 51. The humeral component according to implementation 48 wherein the stemless portion includes a plurality of holes for securing sutures.

Implementation 52. The humeral component according to implementation 48 wherein the stemless portion includes at least one through hole.

Implementation 53. The humeral component according to implementation 48 wherein the proximal portion of the distal end of the stemless portion is cylindrical.

Implementation 54. The humeral component according to implementation 48 wherein the distal portion of the distal end of the stemless portion is flat.

Implementation 55. The humeral component according to implementation 48 wherein the distal surface of the stemless portion is osteoinductive.

Implementation 56. The humeral component according to implementation 48 wherein the stemless portion has a cavity for bone grafting.

Implementation 57. The humeral component according to implementation 48 wherein the distal end of the liner portion is irregularly shaped.

Implementation 58. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion, an insert portion having a proximal end and a distal end, said proximal end adapted to accept a fastener and said distal end adapted for insertion into proximal end of stemless portion, a fastener portion having a proximal end and a distal end, said distal end adapted for insertion into proximal end of stemless portion.

Implementation 59. The humeral component according to implementation 58 wherein the stemless portion includes at least one through hole.

Implementation 60. The humeral component according to implementation 58 wherein the stemless portion includes at least one through slot.

Implementation 61. The humeral component according to implementation 58 wherein the stemless portion accepts an insert for securing into bone.

Implementation 62. The humeral component according to implementation 58 wherein the proximal portion of the distal end of the stemless portion is curved.

Implementation 63. The humeral component according to implementation 58 wherein the distal portion of the distal end of the stemless portion is flat.

Implementation 64. The humeral component according to implementation 58 wherein the distal surface of the stemless portion is osteoinductive.

Implementation 65. The humeral component according to implementation 58 wherein the distal end of the liner portion is irregularly shaped.

Implementation 66. The humeral component according to implementation 58 wherein the distal end of the insert portion is serrated.

Implementation 67. The humeral component according to implementation 66 wherein the insert portion is expandable by outward pressure from a fastener.

Implementation 68. The humeral component according to implementation 58 wherein the fastener has an osteoinductive distal surface.

Implementation 69. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion.

Implementation 70. The humeral component according to implementation 69 wherein the stemless portion is threaded along its surface from its proximal end to its distal end.

Implementation 71. The humeral component according to implementation 70 wherein the distal end of the stemless portion is a different diameter than its proximal end.

Implementation 72. The humeral component according to implementation 69 wherein the proximal portion of the distal end of the stemless portion is curved.

Implementation 73. The humeral component according to implementation 69 wherein the distal surface of the stemless portion is osteoinductive.

Implementation 74. The humeral component according to implementation 69 wherein the stemless portion has a cavity for bone grafting.

Implementation 75. The humeral component according to implementation 69 wherein the distal end of the liner portion is irregularly shaped.

Implementation 76. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion, a fastener portion having a proximal end and a distal end, said distal end adapted for insertion into proximal end of stemless portion.

Implementation 77. The humeral component according to implementation 76 wherein the distal portion of the distal end of the stemless portion is curved.

Implementation 78. The humeral component according to implementation 76 wherein the proximal end of stemless portion includes a plurality of holes for sutures.

Implementation 79. The humeral component according to implementation 76 wherein the stemless portion includes at least one through hole.

Implementation 80. The humeral component according to implementation 76 wherein the stemless portion accepts a fastener to compress stemless portion into bone.

Implementation 81. The humeral component according to implementation 76 wherein the distal surface of the stemless portion is osteoinductive.

Implementation 82. The humeral component according to implementation 76 wherein the distal end of the liner portion is irregularly shaped.

Implementation 83. The humeral component according to implementation 76 wherein the fastener has an osteoinductive distal surface.

Implementation 84. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion.

Implementation 85. The humeral component according to implementation 84 wherein the distal end of stemless portion includes a plurality of fins, each of which is provided with at least one serrated tooth along its surface.

Implementation 86. The humeral component according to implementation 84 wherein the stemless portion includes a plurality of holes for sutures.

Implementation 87. The humeral component according to implementation 84 wherein the proximal portion of the distal end of the stemless portion is curved.

Implementation 88. The humeral component according to implementation 84 wherein the distal portion of the distal end of the stemless portion is curved.

Implementation 89. The humeral component according to implementation 84 wherein the stemless portion has a plurality of slots to allow for cutting of bone along the fins.

Implementation 90. The humeral component according to implementation 84 wherein the distal surface of the stemless portion is osteoinductive.

Implementation 91. The humeral component according to implementation 84 wherein the distal end of the liner portion is irregularly shaped.

Implementation 92. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion.

Implementation 93. The humeral component according to implementation 92 wherein the distal end of stemless portion includes a plurality of fins, each of which is provided with at least one serrated tooth along its surface.

Implementation 94. The humeral component according to implementation 92 wherein the stemless portion includes a plurality of holes for sutures.

Implementation 95. The humeral component according to implementation 92 wherein the proximal portion of the distal end of the stemless portion is curved.

Implementation 96. The humeral component according to implementation 92 wherein the distal portion of the distal end of the stemless portion is curved.

Implementation 97. The humeral component according to implementation 92 wherein the stemless portion has a plurality of slots to allow for cutting of bone along the fins.

Implementation 98. The humeral component according to implementation 92 wherein the distal surface of the stemless portion is osteoinductive.

Implementation 99. The humeral component according to implementation 92 wherein the distal end of the liner portion is irregularly shaped.

Implementation 100. The humeral component according to implementation 92 wherein the liner portion has a plurality of holes to allow for passage of sutures.

Implementation 101. The humeral component according to implementation 92 wherein the liner portion can have a plurality of slots to allow for passage of sutures.

Implementation 102. The humeral component according to implementation 92 wherein the liner portion can have a plurality of slots for attachment of a device to provide compression to liner portion.

Implementation 103. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion.

Implementation 104. The humeral component according to implementation 103 wherein the distal end of stemless portion includes a plurality of fins, each of which is provided with at least one serrated tooth along its surface.

Implementation 105. The humeral component according to implementation 103 wherein the stemless portion includes a plurality of holes for sutures.

Implementation 106. The humeral component according to implementation 103 wherein the stemless portion has a plurality of slots to allow for cutting of bone along the fins.

Implementation 107. The humeral component according to implementation 103 wherein the stemless portion includes a plurality of slots for attachment of a device to provide compression to liner portion.

Implementation 108. The humeral component according to implementation 103 wherein the proximal portion of the distal end of the stemless portion is curved.

Implementation 109. The humeral component according to implementation 103 wherein the distal portion of the distal end of the stemless portion is curved.

Implementation 110. The humeral component according to implementation 103 wherein the stemless portion has a plurality of slots to allow for cutting of bone along the fins.

Implementation 111. The humeral component according to implementation 103 wherein the distal surface of the stemless portion is osteoinductive.

Implementation 112. The humeral component according to implementation 103 wherein the distal end of the liner portion is irregularly shaped.

Implementation 113. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion, an insert portion having a proximal end and a distal end, said proximal end adapted to accept a fastener and said proximal end adapted for insertion into distal end of stemless portion, a fastener portion having a proximal end and a distal end, said distal end adapted for insertion through proximal end of stemless portion.

Implementation 114. The humeral component according to implementation 113 wherein the distal end of stemless portion includes a plurality of fins, each of which is provided with at least one serrated tooth along its surface.

Implementation 115. The humeral component according to implementation 114 wherein the plurality of fins can be of equal length.

Implementation 116. The humeral component according to implementation 114 wherein the plurality of fins can be of different lengths.

Implementation 117. The humeral component according to implementation 114 wherein the plurality of fins can be positioned at equal angles to each other.

Implementation 118. The humeral component according to implementation 114 wherein the plurality of fins can be positioned at different angles to each other.

Implementation 119. The humeral component according to implementation 113 wherein the stemless portion has a plurality of slots to allow for cutting of bone along the fins.

Implementation 120. The humeral component according to implementation 113 wherein the stemless portion accepts an insert portion.

Implementation 121. The humeral component according to implementation 113 wherein the proximal portion of the distal end of the stemless portion is curved.

Implementation 122. The humeral component according to implementation 113 wherein the distal portion of the distal end of the stemless portion is curved.

Implementation 123. The humeral component according to implementation 113 wherein the distal surface of the stemless portion is osteoinductive.

Implementation 124. The humeral component according to implementation 113 wherein the distal end of the liner portion is irregularly shaped.

Implementation 125. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion, a stem portion having a proximal end and a distal end, said proximal end adapted to accept a fastener and said proximal end adapted for insertion into distal end of stemless portion, and said distal end adapted for insertion into the humerus, a fastener portion having a proximal end and a distal end, said distal end adapted for insertion through proximal end of stemless portion.

Implementation 126. The humeral component according to implementation 125 wherein the distal end of stemless portion includes a plurality of fins, each of which is provided with at least one serrated tooth along its surface.

Implementation 127. The humeral component according to implementation 126 wherein the plurality of fins can be of equal lengths.

Implementation 128. The humeral component according to implementation 126 wherein the plurality of fins can be of different lengths.

Implementation 129. The humeral component according to implementation 126 wherein the plurality of fins can be positioned at equal angles to each other.

Implementation 130. The humeral component according to implementation 126 wherein the plurality of fins can be positioned at different angles to each other.

Implementation 131. The humeral component according to implementation 125 wherein the stemless portion has a plurality of slots to allow for cutting of bone along the fins.

Implementation 132. The humeral component according to implementation 125 wherein the proximal portion of the distal end of the stemless portion is curved.

Implementation 133. The humeral component according to implementation 125 wherein the distal portion of the distal end of the stemless portion is curved.

Implementation 134. The humeral component according to implementation 125 wherein the distal surface of the stemless portion is osteoinductive.

Implementation 135. The humeral component according to implementation 125 wherein the stemless portion accepts a stem portion.

Implementation 136. The humeral component according to implementation 125 wherein the stem portion includes at least one slot.

Implementation 137. The humeral component according to implementation 125 wherein the proximal end of the stem portion includes holes for passing of suture.

Implementation 138. The humeral component according to implementation 125 wherein the proximal end of the stem portion is shaped for securing into bone.

Implementation 139. The humeral component according to implementation 125 wherein the distal end of the stem portion is shaped for securing into bone.

Implementation 140. The humeral component according to implementation 125 wherein the proximal surface of the stem portion is osteoinductive.

Implementation 141. The humeral component according to implementation 125 wherein the distal end of the liner portion is irregularly shaped.

Implementation 142. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion, an insert portion having a proximal end and a distal end, said proximal end adapted to accept a fastener and said proximal end adapted for insertion into proximal end of stemless portion, a fastener portion having a proximal end and a distal end, said distal end adapted for insertion through proximal end of stemless portion.

Implementation 143. The humeral component according to implementation 142 wherein the distal end of stemless portion includes a plurality of fins, each of which is provided with at least one serrated tooth along its surface.

Implementation 144. The humeral component according to implementation 143 wherein the plurality of fins can be of equal lengths.

Implementation 145. The humeral component according to implementation 143 wherein the plurality of fins can be of different lengths.

Implementation 146. The humeral component according to implementation 143 wherein the plurality of fins can be positioned at equal angles to each other.

Implementation 147. The humeral component according to implementation 143 wherein the plurality of fins can be positioned at different angles to each other.

Implementation 148. The humeral component according to implementation 143 wherein the plurality of fins can have slots or holes for sutures.

Implementation 149. The humeral component according to implementation 142 wherein the stemless portion has a plurality of slots to allow for cutting of bone along the fins.

Implementation 150. The humeral component according to implementation 142 wherein the stemless portion accepts an insert portion.

Implementation 151. The humeral component according to implementation 149 wherein the distal surface of the insert portion is osteoinductive.

Implementation 152. The humeral component according to implementation 142 wherein the proximal portion of the distal end of the stemless portion is curved.

Implementation 153. The humeral component according to implementation 142 wherein the distal portion of the distal end of the stemless portion is curved.

Implementation 154. The humeral component according to implementation 142 wherein the distal surface of the stemless portion is osteoinductive.

Implementation 155. The humeral component according to implementation 142 wherein the stemless portion has slots to accept a device to provide compression to liner portion.

Implementation 156. The humeral component according to implementation 142 wherein the distal end of the liner portion is irregularly shaped.

Implementation 157. The humeral component according to implementation 142 wherein the liner portion has slots to allow access to a device to provide compression to liner portion.

Implementation 158. The humeral component according to implementation 142 wherein the distal surface of the fastener portion is osteoinductive.

Implementation 159. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion, a stem portion having a proximal end and a distal end, said proximal end adapted to accept a fastener and said proximal end adapted for insertion into distal end of stemless portion, and said distal end adapted for insertion into the humerus, a fastener portion having a proximal end and a distal end, said distal end adapted for insertion through proximal end of stemless portion.

Implementation 160. The humeral component according to implementation 159 wherein the distal end of stemless portion includes a plurality of fins, each of which is provided with at least one serrated tooth along its surface.

Implementation 161. The humeral component according to implementation 160 wherein the plurality of fins can be of equal lengths.

Implementation 162. The humeral component according to implementation 160 wherein the plurality of fins can be of different lengths.

Implementation 163. The humeral component according to implementation 160 wherein the plurality of fins can be positioned at equal angles to each other.

Implementation 164. The humeral component according to implementation 160 wherein the plurality of fins can be positioned at different angles to each other.

Implementation 165. The humeral component according to implementation 160 wherein the plurality of fins can have slots and/or holes for sutures.

Implementation 166. The humeral component according to implementation 159 wherein the stemless portion has a plurality of slots to allow for cutting of bone along the fins.

Implementation 167. The humeral component according to implementation 159 wherein the proximal portion of the distal end of the stemless portion is curved.

Implementation 168. The humeral component according to implementation 159 wherein the distal portion of the distal end of the stemless portion is curved.

Implementation 169. The humeral component according to implementation 159 wherein the distal surface of the stemless portion is osteoinductive.

Implementation 170. The humeral component according to implementation 159 wherein the stemless portion accepts a stem portion.

Implementation 171. The humeral component according to implementation 159 wherein the stemless portion has slots to accept a device to provide compression to liner portion.

Implementation 172. The humeral component according to implementation 159 wherein the stem portion includes at least one slot.

Implementation 173. The humeral component according to implementation 159 wherein the proximal end of the stem portion includes holes for passing of suture.

Implementation 174. The humeral component according to implementation 159 wherein the proximal end of the stem portion is shaped for securing into bone.

Implementation 175. The humeral component according to implementation 159 wherein the distal end of the stem portion is shaped for securing into bone.

Implementation 176. The humeral component according to implementation 159 wherein the proximal surface of the stem portion is osteoinductive.

Implementation 177. The humeral component according to implementation 159 wherein the distal end of the liner portion is irregularly shaped.

Implementation 178. The humeral component according to implementation 159 wherein the liner portion has slots to allow access to a device to provide compression to liner portion.

Implementation 179. A humeral component of a prosthetic assembly for use in repairing or reconstructing a joint between a humerus and a scapula by cooperating with a socket portion of a glenoid component that is adapted for attachment to the scapula, said humeral component comprising a spacer portion having a proximal end and a distal end, said distal end of said spacer portion being adapted to accept the proximal end of the stem portion, a dual tapered portion having a proximal end and a distal end, said proximal end of said dual tapered portion being adapted to attach to the distal cavity of the hemispherical portion, and said distal portion of said dual tapered portion being adapted to attach to the proximal cavity of the spacer portion, a hemispherical portion having a proximal end and a distal end, said proximal end being adapted to interface with a socket portion of a glenoid component, and said distal end of said hemispherical portion being adapted to attach to the proximal tapered end of the dual tapered portion, a stem portion having a proximal end and a distal end, said proximal end adapted to accept a fastener and said proximal end adapted for insertion into distal end of spacer portion, and said distal end adapted for insertion into the humerus, a fastener portion having a proximal end and a distal end, said distal end adapted for insertion through proximal end of stemless portion.

Implementation 180. The humeral component according to implementation 179 wherein the distal end of hemispherical portion includes a tapered cavity.

Implementation 181. The humeral component according to implementation 179 wherein the proximal end of spacer portion can accept a tapered component.

Implementation 182. The humeral component according to implementation 179 wherein spacer portion can attach at different angles.

Implementation 183. The humeral component according to implementation 179 wherein dual tapered portion can have a through hole for passage of a fastener.

Implementation 184. The humeral component according to implementation 179 wherein the stem portion includes at least one cavity.

Implementation 185. The humeral component according to implementation 179 wherein the proximal end of the stem portion includes holes for passing of suture.

Implementation 186. The humeral component according to implementation 179 wherein the proximal end of the stem portion is shaped for securing into bone.

Implementation 187. The humeral component according to implementation 179 wherein the distal end of the stem portion is shaped for securing into bone.

Implementation 188. The humeral component according to implementation 179 wherein the proximal surface of the stem portion is osteoinductive.

Implementation 189. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion, a fastener portion having a proximal end and a distal end, said distal end adapted for insertion through proximal end of stemless portion.

Implementation 190. The humeral component according to implementation 189 wherein the distal end of stemless portion includes a plurality of ribs.

Implementation 191. The humeral component according to implementation 190 wherein the plurality of ribs can be of equal lengths.

Implementation 192. The humeral component according to implementation 190 wherein the plurality of ribs can be of different lengths.

Implementation 193. The humeral component according to implementation 190 wherein the plurality of ribs can be positioned at equal angles to each other.

Implementation 194. The humeral component according to implementation 190 wherein the plurality of ribs can be positioned at different angles to each other.

Implementation 195. The humeral component according to implementation 189 wherein the stemless portion accepts a plurality of fastener portions.

Implementation 196. The humeral component according to implementation 189 wherein the proximal portion of the distal end of the stemless portion is curved.

Implementation 197. The humeral component according to implementation 189 wherein the distal portion of the distal end of the stemless portion is curved.

Implementation 198. The humeral component according to implementation 189 wherein the distal surface of the stemless portion is osteoinductive.

Implementation 199. The humeral component according to implementation 189 wherein the distal surface of the stemless portion is osteoinductive.

Implementation 200. The humeral component according to implementation 189 wherein the distal end of the liner portion is irregularly shaped.

Implementation 201. The humeral component according to implementation 189 wherein the distal surface of the fastener portion is osteoinductive.

Implementation 202. The humeral component according to implementation 189 wherein the fastener portions can be of the same length.

Implementation 203. The humeral component according to implementation 189 wherein the fastener portions can be of different lengths.

Implementation 204. The humeral component according to implementation 189 wherein the fastener portions can be threaded.

Implementation 205. The humeral component according to implementation 189 wherein the fastener portions can be smooth.

Implementation 206. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion, a plate portion that is adapted to be in contact with the humerus, said plate portion being attached to the distal end of the stemless portion, a fastener portion having a proximal end and a distal end, said distal end adapted for insertion through proximal end of stemless portion.

Implementation 207. The humeral component according to implementation 206 wherein the distal end of stemless portion includes a plurality of ribs.

Implementation 208. The humeral component according to implementation 206 wherein the plurality of ribs can be of equal lengths.

Implementation 209. The humeral component according to implementation 206 wherein the plurality of ribs can be of different lengths.

Implementation 210. The humeral component according to implementation 206 wherein the plurality of ribs can be positioned at equal angles to each other.

Implementation 211. The humeral component according to implementation 206 wherein the plurality of ribs can be positioned at different angles to each other.

Implementation 212. The humeral component according to implementation 205 wherein the stemless portion accepts a plurality of fastener portions.

Implementation 213. The humeral component according to implementation 205 wherein the proximal portion of the distal end of the stemless portion is curved.

Implementation 214. The humeral component according to implementation 205 wherein the distal portion of the distal end of the stemless portion is curved.

Implementation 215. The humeral component according to implementation 205 wherein the distal surface of the stemless portion is osteoinductive.

Implementation 216. The humeral component according to implementation 205 wherein the distal end of the liner portion is irregularly shaped.

Implementation 217. The humeral component according to implementation 205 wherein the distal surface of the fastener portion is osteoinductive.

Implementation 218. The humeral component according to implementation 205 wherein the fastener portions can be of the same length.

Implementation 219. The humeral component according to implementation 205 wherein the fastener portions can be of different lengths.

Implementation 220. The humeral component according to implementation 205 wherein the fastener portions can be threaded.

Implementation 221. The humeral component according to implementation 205 wherein the fastener portions can be smooth.

Implementation 222. The humeral component according to implementation 205 wherein the plate portion includes a plurality of holes to accept fasteners.

Implementation 223. The humeral component according to implementation 205 wherein the plate portion includes a plurality of holes at different angles.

Implementation 224. The humeral component according to implementation 205 wherein the plate portion includes an osteoinductive surface.

Implementation 225. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion, a ring portion having a proximal end and a distal end, said distal end adapted for insertion into proximal end of stemless portion.

Implementation 226. The humeral component according to implementation 224 wherein the distal portion of the distal end of the stemless portion is curved.

Implementation 227. The humeral component according to implementation 224 wherein the proximal end of stemless portion includes a plurality of holes for sutures.

Implementation 228. The humeral component according to implementation 224 wherein the distal end of stemless portion includes a plurality of fins, each of which can be provided with at least one serrated tooth along its surface.

Implementation 229. The humeral component according to implementation 227 wherein the plurality of fins can be of equal lengths.

Implementation 230. The humeral component according to implementation 227 wherein the plurality of fins can be of different lengths.

Implementation 231. The humeral component according to implementation 227 wherein the plurality of fins can be positioned at equal angles to each other.

Implementation 232. The humeral component according to implementation 227 wherein the plurality of fins can be positioned at different angles to each other.

Implementation 233. The humeral component according to implementation 227 wherein the plurality of fins can be curved.

Implementation 234. The humeral component according to implementation 224 wherein the distal surface of the stemless portion is osteoinductive.

Implementation 235. The humeral component according to implementation 224 wherein the ring portion has holes to accept a device for deployment.

Implementation 236. The humeral component according to implementation 224 wherein the proximal end of the ring portion can be tapered.

Implementation 237. The humeral component according to implementation 224 wherein the distal end of the liner portion can be slotted to accept the ring portion.

Implementation 238. The humeral component according to implementation 224 wherein the distal end of the liner portion is irregularly shaped.

Implementation 239. A humeral component of a prosthetic assembly for use in repairing or reconstructing a joint between a humerus and a scapula by cooperating with a ball portion of a glenoid component that is adapted for attachment to the scapula, said humeral component comprising a stemless portion having a proximal end and a distal end, said distal end of said stemless portion being adapted for insertion into the humerus, a liner portion that is adapted to receive the ball portion of the glenoid component, said liner portion being attached to the proximal end of the stemless portion.

Implementation 240. The humeral component according to implementation 238 wherein the distal portion of the distal end of the stemless portion is curved.

Implementation 241. The humeral component according to implementation 238 wherein the distal end of stemless portion includes a plurality of fins, each of which can be provided with at least one serrated tooth along its surface.

Implementation 242. The humeral component according to implementation 238 wherein the plurality of fins can be of equal length.

Implementation 243. The humeral component according to implementation 238 wherein the plurality of fins can be of different lengths.

Implementation 244. The humeral component according to implementation 238 wherein the plurality of fins can be of equal height.

Implementation 245. The humeral component according to implementation 238 wherein the plurality of fins can be of different heights.

Implementation 246. The humeral component according to implementation 238 wherein the plurality of fins can be positioned at equal angles to each other.

Implementation 247. The humeral component according to implementation 238 wherein the plurality of fins can be positioned at different angles to each other.

Implementation 248. The humeral component according to implementation 238 wherein the plurality of fins can be curved with a single radius.

Implementation 249. The humeral component according to implementation 238 wherein the plurality of fins can be curved with a plurality of radii.

Implementation 250. The humeral component according to implementation 238 wherein the distal surface of the stemless portion is osteoinductive.

Implementation 251. The humeral component according to implementation 238 wherein the distal end of the liner portion is irregularly shaped.

One or more elements or aspects or steps, or any portion(s) thereof, from one or more of any of claims 1 to 12 below can be combined with one or more elements or aspects or steps, or any portion(s) thereof, from one or more of any of the other claims 13 to 178 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A humeral component of a prosthetic assembly for use in repairing or reconstructing a joint between a humerus and a scapula by cooperating with a ball portion of a glenoid component that is adapted for attachment to the scapula, said humeral component comprising:
a stemless portion having a proximal end and a distal end, said distal end of said stemless portion being adapted for insertion into the humerus;
a liner portion that is adapted to receive the ball portion of the glenoid component, said liner portion being attached to the proximal end of the stemless portion;
an insert portion having a proximal end and a distal end, said proximal end adapted to accept a fastener and said proximal end adapted for insertion into distal end of stemless portion;
a fastener portion having a proximal end and a distal end, said distal end adapted for insertion through proximal end of stemless portion.

2-8. (canceled)

9. The humeral component of claim 1, wherein the proximal portion of the distal end of the stemless portion is curved.

10. The humeral component of claim 1, wherein the distal portion of the distal end of the stemless portion is curved.

11-25. (canceled)

26. The humeral component of claim 1, wherein the proximal portion of the distal end of the stemless portion is cylindrical.

27. The humeral component of claim 1, wherein the distal portion of the distal end of the stemless portion is flat.

28-34. (canceled)

35. The humeral component of claim 1, wherein the distal portion of the distal end of the stemless portion is cylindrical.

36-177. (canceled)

178. A humeral component of a prosthetic assembly for use in repairing or reconstructing a joint between a humerus and a scapula by cooperating with a ball portion of a glenoid component that is adapted for attachment to the scapula, said humeral component comprising:
a stemless portion having a proximal end and a distal end, said distal end of said stemless portion being adapted for insertion into the humerus;
a liner portion that is adapted to receive the ball portion of the glenoid component, said liner portion being attached to the proximal end of the stemless portion; and
an insert portion having a proximal end and a distal end, said proximal end adapted to accept a fastener and said proximal end adapted for insertion into distal end of stemless portion.

179. The humeral component of claim 178, wherein the distal end of stemless portion includes a plurality of fins, each of which is provided with at least one serrated tooth along its surface.

180. The humeral component of claim 179, wherein the plurality of fins can be of equal lengths.

181. The humeral component of claim 179, wherein the plurality of fins can be of different lengths.

182. The humeral component of claim 179, wherein the plurality of fins can be positioned at equal angles to each other.

183. The humeral component of claim 179, wherein the plurality of fins can be positioned at different angles to each other.

184. The humeral component of claim 184, wherein the distal end of the insert portion is serrated.

185. The humeral component of claim 184, wherein the insert portion is expandable by outward pressure from the fastener.

186. A humeral component of a prosthetic assembly for use in repairing or reconstructing a joint between a humerus and a scapula by cooperating with a ball portion of a glenoid component that is adapted for attachment to the scapula, said humeral component comprising:
a stemless portion having a proximal end and a distal end, said distal end of said stemless portion being adapted for insertion into the humerus; and
a liner portion that is adapted to receive the ball portion of the glenoid component, said liner portion being attached to the proximal end of the stemless portion;
wherein the liner portion has a plurality of holes or slots.

187. The humeral component of claim 186, wherein the stemless portion has at least one through slot.

188. The humeral component of claim 186, wherein the stemless portion includes at least one through hole.

189. The humeral component of claim 186, wherein the stemless portion has a cavity for bone grafting.

190. The humeral component of claim 186, wherein the distal surface of the stemless portion is osteoinductive.

191. The humeral component of claim 186, wherein the distal end of the liner portion is irregularly shaped.
